(19) United States
(12) Patent Application Publication
(10) Pub. No.: US 2004/0010118 A1
(43) Pub. Date: Jan. 15, 2004

(54) Novel proteins and nucleic acids encoding same

(76) Inventors: Bryan D. Zerhusen, Branford, CT (US); Muralidhara Padigaru, Branford, CT (US); Kimberly Spytek, New Haven, CT (US); Steven Spaderna, Berlin, CT (US); Esha A. Gangolli, Branford, CT (US); Luca Rastelli, Guilford, CT (US); Catherine E. Burgess, Wethersfield, CT (US); Kumud Majumder, Stamford, CT (US); Richard Shimkets, West Haven, CT (US); Vishnu Mishra, Branford, CT (US); Corine Vernet, North Branford, CT (US); Edward S. Szekeres, Branford, CT (US); William M. Grosse, Branford, CT (US); John P. Alsobrook II, Madison, CT (US); Xiaohong Liu, Branford, CT (US); Valerie L. Gerlach, Branford, CT (US); Karen Ellerman, Branford, CT (US); Glennda Smithson, Branford, CT (US); John Peyman, New Haven, CT (US); David Stone, Guilford, CT (US); John MacDougall, Hamden, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 09/930,512

(22) Filed: Aug. 15, 2001

Related U.S. Application Data
(60) Provisional application No. 60/225,692, filed on Aug. 16, 2000.
(60) Provisional application No. 60/225,693, filed on Aug. 16, 2000.
(60) Provisional application No. 60/225,837, filed on Aug. 16, 2000.
(60) Provisional application No. 60/226,236, filed on Aug. 18, 2000.
(60) Provisional application No. 60/226,353, filed on Aug. 18, 2000.
(60) Provisional application No. 60/227,085, filed on Aug. 22, 2000.
(60) Provisional application No. 60/227,395, filed on Aug. 23, 2000.
(60) Provisional application No. 60/227,492, filed on Aug. 24, 2000.
(60) Provisional application No. 60/227,600, filed on Aug. 24, 2000.
(60) Provisional application No. 60/275,952, filed on Mar. 14, 2001.

(51) Int. Cl. C07K 14/435; C07H 21/04

(52) U.S. Cl. 530/350; 536/23.5

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. S. No. 60/225,692, filed Aug. 16, 2000; U.S. S. No. 60/225,693, filed Aug. 16, 2000; U.S. S. No. 60/225,837, filed Aug. 16, 2000; U.S. S. No. 60/226,236, filed Aug. 16, 2000; U.S. S. No. 60/226,353, filed Aug. 18, 2000; U.S. S. No. 60/227,235, filed Aug. 22, 2000; U.S. S. No. 60/227,395, iled Aug. 23, 2000; U.S. S. No. 60/227,492, filed Aug. 2, 2000; U.S. S. No. 60/227,600, filed Aug. 24, 2000; U.S. S. No. 60/275,952, filed Mar. 3, 2001, each of which is incorporated by reference in its entirety.

### FIELD OF THE INVENTION

The invention generally relates to nucleic acids and polypeptides encoded thereby.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vector, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8 and NOV9 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, and 19. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18 and 20. The nucleic acid can be, for example, a genomic DNA fragment or a cDNA molecule that includes the nucleic acid sequence of any of SEQ ID NOS:1, 3, 5, 7, 9, 11, 13, 15, 17, and 19.

Also included n the invention is an oligonucleotide, e.g., an oligonucleotide which includes at least 6 contiguous nucleotides of a NOVX nucleic acid (e.g., SEQ ID NOS:1, 3, 5, 7, 9, 11, 13, 15, 17 and 19) or a complement of said oligonucleotide.

Also included in the invention are substantially purified NOVX polypeptides (SEQ ID NOS:2, 4, 6, 8, 10, 12, 14, 16, 18 and 20). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or mote containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, is further described herein.

Also within the scope of the invention is the use of a therapeutic in the manufacture of a medicament for treating or preventing disorders or syndromes including, e.g., cancer, bone cancer; bone disorders, osteoporosis, osteopetrosis, arthritis, osteomyelitis, osteonecrosis, avascular necrosis, Paget's Disease; hematopoietic disorders, Spinal Diseases, immune disorders, regeneration (in vitro and m vivo), Endometriosis, Diabetes, Autoimmune disease, Renal artery stenosis, Interstitial nephritis, Glomerulonephritis Polycystic kidney disease, viral/bacterial/parasitic infections, antiviral and antitumor immune responses, inflammation and acute phase responses, cell proliferation regulation, systemic juvenile rheumatoid arthritis, atherosclerosis, Multiple sclerosis, systemic lupus erythematosus, asthma, emphysema, scleroderma, allergy, ARDS, Hirschsprung's disease, Crohn's disease, appendicitis, inflammatory bowel disease, diverticular disease, melanoma, Wilm's tumor, rhabdomyosarcomas cancer, hemophilia, hypercoagulation, cardiovascular disorders, restenosis, idiopathic thrombocytopenic purpura, allergies, immunodeficiencics, transplantation, graft versus host disease (GVHD), lymphaedema, fertility disorders, growth disorders, regulatory disorders, developmental disorders, Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyban syndrome, ataxia-telangiectasia, leukodystrophies behavioral disorders, addiction, anxiety, pain, neuroprotection, ocular disorders, glioblastoma, glioma, uterine tumors, melanoma, bladder tumors, lung tumors, HCV infection, Burkitt Lymphoma, metastatic tumors, immunological disorders particularly those involving T-cells, Episodic Ataxia, type 1, Long QT Syndrome 1 and 2, Benign Neonatal Epilepsy, Jervell and Lange-Neilson syndrome, Autosomal dominant deafness (DFNA 2), non-insulin dependent diabetes mellitus, CNS disorders, arrhythmia, seizure, hypertension therapy, renal tubular acidosis, IgA and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a rest compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invest Lion includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (Polypeptide) | Homology |
| 1 | GMba261a [symbol] A | 1 | 2 | Asparaginyl Endopeptidase-like |
| 2 | Spec_000-392 | 3 | 4 | Tyrosyl-tRNA Synthetase-like |
| 3 | 32073570_EXT | 5 | 6 | Melastatin-like |
| 4 | 124141642_EXT | 7 | 8 | Leucine-Rich Repeat-like |
| 5 | GM_51624520A1/dj1160k | 9 | 10 | CD-81/Tetraspanin-like |
| | [symbol] _A1 | | | |
| 6A | GM_AC011898_A | 11 | 12 | Voltage-Dependent Anion |
| | | | | Channel-like |
| 6B | GM_AL133368_A | 13 | 14 | Voltage-Dependent Anion |
| | | | | Channel-like [symbol] |
| 7 | AC016572_dal | 15 | 16 | Butyrophilin Receptor-like |
| 8 | 101360122_EXT4 | 17 | 18 | MEGF/Fibrillin-like |
| 9 | GMG55707_EXT.0.1_dal | 19 | 20 | Growth/Differentiation Factor |
| | | | | 6-like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to a Asparaginyl Endopeptidase-like family of proteins. Thus, the NOV1 nucleic acids, polypeptides, antibodies and related compounds according to the invention will be useful in therapeutic and diagnostic applications implicated in, for example; various cancers, bone disorders, osteoporosis, arthritis, hematopoietic disorders, spinal Diseases, immune disorders, regeneration (in vitro and in vivo), Endometriosis, Fertility, Diabetes, Autoimmune Disease, viral/bacterial/parasitic infections, and/or other pathologies/disorders.

NOV2 is homologous to the Tyrosyl-tRNA Synthetase-like family of proteins. Thus NOV2 nucleic acids, polypeptides, antibodies and related compounds according to the invention will be useful in therapeutic and diagnostic applications implicated in, for example; antiviral and antitumor immune responses, inflammation and acute phase responses cell proliferation regulation, systemic juvenile rheumatoid arthritis, atherosclerosis, Multiple sclerosis, Osteopetrosis and/or other pathologies/disorders.

NOV3 is homologous to a family of Melastatin-like proteins. Thus, the NOV3 nucleic acids and polypeptides, antibodies and related compounds according to the invention will be useful in therapeutic and diagnostic applications implicated in, for example; systemic lupus erythematosus, autoimmune disease, asthma, emphysema, scleroderma, allergy, ARDS, Hirschsprung's disease, Crohn's disease, appendicitis, inflammatory bowel disease, diverticular disease, melanoma, Wilm's tumor, rhabdomyosarcomas cancer, hemophilia, hypercoagulation, carciovascular disorder s, restenosis, idiopathic thrombocytopenic purpura, allergies, immunodeficiencies, transplantation, graft versus host disease (GVHD), lymphaedema, fertility disorders, growth disorders, regulatory disorders, and developmental disorders and/or other pathologies/disorders.

NOV4 is homologous to the Leucine-Rich Repeat-like family of proteins. Thus, NOV4 nucleic acids, polypeptides, antibodies and related compounds according to the invention will be useful in therapeutic and diagnostic applications implicated in, for example; Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies behavioral disorders, addiction, anxiety, pain, neuroprotection, systemic lupus erythematosus, autoimmune disease, asthma, emphysema, scleroderma, allergy, ARDS, fertility, ocular disorders, glioblastoma, glioma, uterine tumors, melanoma, bladder tumors, lung tumors and/or other pathologies/disorders.

NOV5 is homologous to the CD-81/Tetraspanin-like family of proteins. Thus NOV5 nucleic acids, polypeptides, antibodies and related compounds according to the invention will be useful in therapeutic and diagnostic applications implicated in, for example; HCV infection, Burkitt Lymphoma, and metastatic tumors, immunological disorders particularly those involving T-cells and/or other pathologies/disorders.

NOV6a and NOV6b are homologous to the Voltage-Dependent Anion Channel-like family of proteins. Thus NOV6 nucleic acids, polypeptides, antibodies and related compounds according to the invention will be useful in therapeutic and diagnostic applications implicated in, for example; Episodic Ataxia, type 1, Long QT Syndrome 1 and 2, Benign Neonatal Epilepsy, Jervell and Lange-Neilson syndrome, Autosomal dominant deafness (DFNA 2), non-insulin dependent diabetes mellitus, CNS disorders, arrhythmia, seizure, asthma, hypertension therapy and/or other pathologies/disorders.

NOV7 is homologous to members of the Butyrophilin Receptor-like family of proteins. Thus, the NOV7 nucleic acids, polypeptides, antibodies and related compounds according to the invention will be useful in therapeutic and diagnostic applications implicated in, for example; Fertility, Inflammatory bowel disease, Diverticular disease, Autoimmune disorders and Cancer and/or other pathologies/disorders.

NOV8 is homologous to the MEGF/Fibrillin-like family of proteins. Thus, NOV8 nucleic acids and polypeptides, antibodies and related compounds according to the invention will be useful in therapeutic and diagnostic applications implicated in, for example; diabetes, autoimmune disease, renal artery stenosis, interstitial nephritis, glomerulonephritis, polycystic kidney disease, systemic lupus erythematosus, renal tubular acidosis, IgA nephropathy, hypercalceimia and Lesch-Nyhan syndrome and/or other pathologies/disorders.

NOV9 is homologous to the Growth/Differentiation Factor 6-like family of proteins. Thus, NOV9 nucleic acids and polypeptides, antibodies ank related compounds according to the invention will be useful in therapeutic and diagnostic applications implicated in growth and differentiation disorders and diseases and/or other various pathologies and disorders.

The NOVX nucleic acids and polypeptides can also be used to screen for molecules, which inhibit or enhance NOVX activity or function. Specifically, the nucleic acids and polypeptide according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

A disclosed NOV1 nucleic acid of 0.336 nucleotides (also referred to as GMba261a1_A) encoding a novel asparaginyl endopeptidase-like protein is shown in Table 1A. An open reading frame was identified beginning with an ATG initiation codon at nucleotides 2-4 and ending with a TGA codon at nucleotides 1307-1309. A putative untranslated region upstream from the initiation codon and downstream from the termination codon is underlined in Table 1A. The start and stop codons are in bold letters.

TABLE 1A
NOV1 nucleotide sequence.

| | |
|---|---|
| AATGCTTTGGAAAGTAGCTGTATTCCTCAGCGTGGCACTGGGCACTGGTGCTGTTCCCATAGATG | (SEQ ID NO:1) |
| ATCCTGAAGATGGACGCAAGCACTGGGTGGTGATCGTGGCGGGTTCAAATGGCTGGTATAATTAC | |
| AGGCACCAGGCAGCTGCGTGCCATGCCTACCAGATCATTTACTGGAATGGGATTCCAGACGAGCA | |
| CATCATTGTTATCATCTACGATGACACTCCTCACTCTGAAGACAATCCCACTCCAGGAATTGTAG | |
| TCAACAGACCCAATGGCACGGATGTCTATCAGGGGATTCCCTATTTTCTTTTTACACTGGAGAGG | |
| ATGTTGCCCCGGGGGAATTTCCTTCCTGTGTTGACAGGCGATGCAGAAGCAGTGAAGGGCATAGG | |
| ACAAGUCAAAAACATAAAGAGCGGTCCCCAAAAAAAAGTGTTCGTTTACTTCACTGACCATGGAT | |
| CTACTGGAATACTCGTTTTTCCCAATGAAGATCTTCATGTAAAGTACCTGAATGAGACCATCCAT | |
| TACATGTACATACATAAAATGTACCAAAAGATGGTGTTCTATATTGAAGCCTGTGAGTCTGGGTC | |
| CATGATGAACCACCTGGCTGGTGATACTAATGTTTATGCAACTACTGCTGCCAACCCCAGAGAGT | |
| CGTCCTACACCTGTTACTATGATGAGAAGAGGTCGACGTACCTGGGGGACTGGTACAGCGTCAAC | |
| TGGATGGAAGACTCGGACGTGGAAGATCTGACTAACCAGACCCTGCACAAGCAGTGCCGCCTGGT | |
| AAAATCATACACCAATACCAGCCACATCATGCAGTACGGAAACGAAACGATCTCCACATTAAAAG | |
| TGATGCAGTTTCAGAGTATGAAACACAAAGCCAGTTCTCCTATCTCCCTGCCTCCAGTCACACAC | |
| CTTGACCTCACCCCCAGCCCTGATGTGCCCCTCATGATCGTGAAAAGGAAACTGATGAACACCAA | |
| CGATCTGGAGGACTCCAGGCAGCTCACAGAGGAGATCCAGCGGCATCTGGATGCCAGGCACCTCA | |
| TTGAGAAGTCAGTGCGCAAGATCGCCTCCTTGCTGGCAGCGTCCGAGGCTGAGGTGGAGCAGCTC | |
| CTGTCTGAGAGAGCCCCGTTCACGGGGCATAGCTGCTACCTGGAGGCCCTGCTGCACTTCCAGAC | |
| CCACTGCTTCAACTGGCACTCCCCCACGTGCGAGTATGCGTTGAGACATTTGTACGTGCTGGCCA | |
| ACCTTTGTGAGAAACCGTATCCGCTTCACAGGATAAAATTGTCCATGGACCACGTGTGCCTCGGT | |
| CGCTACTGAAGAGCTGCCTCCTGGAAGCTTTTCCAA | |

The NOV1 nucleic acid was identified on chromosome 13 by TblastN using CuraGen Corporation's sequence file for asparaginyl endopeptidase as run against the Genomic Daily Files made available by GenBank or from files downloaded from the individual sequencing centers. The nucleic acid sequence was predicted from the genomic file ba261a1 by homology to a known asparaginyl endopeptidase. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

In a search of public sequence databases, the NOV1 nucleic acid sequence has 1239 of 1351 bases (91%) identical to Homo sapiens, Legumain mRNA (GENBANK-ID: HSLEGUMAI)(E=3.4e−²⁵²). Public nucleotide databases include all GenBank databases and the GeneSeq patent database.

In all BLAST alignments herein, the “E-value” or “Expect” value is a numeric indication of the probability that the aligned sequences could have achieved meir similarity to the BLAST query sequence by chance alone, within the database that was searched. For example, the probability that the subject (“Sbjct”) retrieved from the NOV1 BLAST analysis, e.g., Homo sapiens Legumain mRNA, matched the Query NOV1 sequence purely by chance is 3.4e−²⁵². The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. Wootton and Federhen, Methods Enzymol 266:554-571, 1996.

The disclosed NOV1 polypeptide (SEQ ID NO:2) encoded by SEQ ID NO:1 has 435 amino acid residues and is presented in Table 1B using the one-letter amino acid code. Signal P, Psort and/or Hydropathy results predict that NOV1 has a signal peptide and is likely to be localized extracellularly with a certainty of 0. 5040. The most likely cleavage site for a NOV1 peptide is between amino acids 17 and 18, at: TGA-VP. NOV1 has a molecular weight of 49787.3 Daltons.

TABLE 1B
Encoded NOV1 protein sequence.

| | |
|---|---|
| MLWKVAVFLSVALGTGAVPIDDPEDGRKHWVVIVAGSNGWYNYRHQAAAAHAYQIIYWNGIPDE | (SEQ ID NO:2) |
| IGQGKNIKSGPQKKVFVYFTDHGSTGILVFPNEDLHVKYLNETIHYMYIHKMYQKMVFYIEACE | |
| SGSMNNHLPODTNVYATTAANPRESSYTCYYDEKRSTYLGDWYSVNWHEDSDVEDLTNQTLHKQ | |
| CRLVKSYTNTSHIMQYGNETISTLKVMQFQSMKHKASSPISLPPVTHLDLTPSPDVPLMIVKRK | |
| LMNTNDLEDSRQLTEEIQRHLDARHLIEKSVRKIASLLAASEAEVEQLLSERAPFTGHSCYLEA | |
| LLHFQTHCFNWHSPTCEYALRHLYVLANLCEKPYPLOIKLSMDHVCLGRY | |

A search of public sequence databases reveals that the NOV1 amino acid sequence has 377 of 435 amino acid residues (86%) identical to, and 398 of 435 residues (91%) positive with, the 433 amino acid residue Legumain protein from Homo Sapiens (ptnr:SPTREMBL-ACC:Q99538)(E=1.1e−²⁰⁷). The global sequence homology (as defined by FASTA alignment with the full length sequence of this protein) is 90.069% amino acid homology and 87.067% amino acid identity. Public amino acid databases include the GenBank databases, SwissProt, PDB and PIR.

The disclosed NOV1 polypeptide has homology to the amino acid sequences shown in the BLASTP data listed in Table 1C.

TABLE 1C
BLAST results for NOV1

| Gene Index/ | | Length | Identity | Positives | |
|---|---|---|---|---|---|
| Identifier | Protein/Organism | (aa) | (%) | (%) | Expect |
| gi\|5031991\|ref\|NP_ | protease, | 433 | 377/435 | 398/435 | 0.0 |
| 005597.1\| | cysteine, 1 | | (86%0 | (90%) | |
| | (legumain) [Homo | | | | |
| | sapiens] | | | | |
| gi\|13111750\|gb\| | protease, | 433 | 376/435 | 398/435 | 0.0 |
| AAH03061.1\|AAH03061 | cysteine, 1 | | (86%) | (91%) | |
| (BC003061) | (legumain) [Homo | | | | |
| | sapiens] | | | | |
| gi\|1890050\|dbj\| | cysteine protease | 433 | 376/435 | 397/435 | 0.0 |
| BAA09530.1\| (D55696) | [Homo sapiens] | | (86%) | (90%) | |
| gi\|13648864\|ref\| | protease, | 401 | 344/399 | 365/399 | 0.0 |
| XP_007399.2\| | cysteine, 1 | | (86%) | 9(91%) | |
| | (legumain) [Homo | | | | |
| | sapiens] | | | | |
| gi\|7242187\|ref\| | protease, | 435 | 326/437 | 370/437 | 0.0 |
| NP_035305.1\| | cysteine, 1; | | (74%) | (84%) | |
| | preprolegumain | | | | |
| | [Mus musculus] | | | | |

The homology between these and other sequences is shown graphically in the ClustalW analysis shown in Table 1D. In the ClustalW alignment of the NOV1 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV1 as disclosed in Tables 1E, were collected from the Conserved Domain Database (CDD) with Reverse Position Specific BLAST analyses. This BLAST analysis software samples domains found in the Smart and Pfam collections. For Table 1E and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, IIY, FYW.

Table 1E lists the domain description from DOMAIN analysis results against NOV1. This indicates that the NOV1 sequence has properties similar to those of other proteins known to contain this domain.

TABLE 1E
Domain Analysis of NOV1

gnl|Pfam|pfam01650, Peptidase_Cl3, Peptidase Cl3 family. (SEQ ID NO:26)
Length = 336 residues, 99.7% aligned
Score = 352 bits (902), Expect = 3e−98

| | | | |
|---|---|---|---|
| NOV1: | 6 | AVFIGVALGTGAVP----IDDPEDGRK------HWVVIVAGSNGWYNYRHQAAACHAYQI | 55 |
| | | \|\|\|\| \| \| +\| \| \| +\| +\| \|+\|\|\|\|\|\|\|+\|\|\|\|\|\| \|\|\|\|\| | |
| Pfam01650: | 1 | AVFLLVVLLIFSVDGADVISLPSEGVTDDGHTNNWAVLVAGSNGWFNYRHQADVCHAYQS | 60 |
| NOV1: | 56 | IYWNGIPDEHIIVMMYDDTAHSEDNPTPGIVINRPNGTDVYQGIPYFLFTLERMLPRGNF | 115 |
| | | + \|\|\|\|\|+\|\|\|\|\|\|\|\| \| + \|\|\|\|\| \|\|\| \|+\| \|\|\| \|+ + + \| | |
| Pfam01650: | 61 | LKPLGIPDENIIVMMYDDIACNARNPRPGTVINHPHGDDVYGGVE-VDYRGYEVTVE-NF | 118 |
| NOV1: | 116 | LPVLTGDAEAVKGIGQGKNIKSGPQKKVFVYFTDHGSTGILVFPN-EDLHVKYLNETIHY | 174 |
| | | \| \|\|\|\| \|\|\| \|\| + \| \| +\|+\|+\|\|\|\| \| \| \|\|+ \|+\|+ \| \| + + | |
| Pfam01650: | 119 | LRVLTGRKEAVT--PGGKVLLSDPNDHIFIYYTDHGGPGFLKFPDSEELYAKDLADALKQ | 176 |
| NOV1: | 175 | MYIHKMYQKMVFYIEACESGSMMNHLPGDTNVYATTAANPRESSYTCYYD---EKESTYL | 231 |
| | | \|+ \|+++\|\|\|+\|\|\|\|\|\|\|\| \| \|\|+\|\|\|\|\|+\| \|\|\|\|+ \| \| \| \| | |
| Pfam01650: | 177 | MHEKGRYKELVFYVEACESGSMFEGLLSPLNIYATTASNAGESSYSHYCDGDIGVYVTDL | 236 |
| NOV1: | 232 | GDWYSVNWMEDSDVEDLTNQTLHKQCRLVKSYTNTSHIMQYGNETISTLKVMQFQSMKHK | 291 |
| | | \|\| \|\|+ \|\|\|\|\| +\|+ +\|\| +\| + \|\| \| \|\|+\| \|\|+ \| \|++ + | |
| Pfam01650: | 237 | GDLYSLAWMEDSEKHNLSKETLQQQYQSVKKRTCLSHVMVYGDLYIRDPKLVLYTGFFGA | 296 |
| NOV1: | 292 | ASSPI-SLPPVTHLDLTPSPDVPLMIVKRKLMNTNDLED | 329 |
| | | + \| \|\| \| \|++ \| \|+ + \|\| \|+ + | |
| Pfam01650: | 297 | VRNTIHDEPPRTPKDVSNQRDADLLTLWRKYRLANNGLE | 335 |

Other BLAST results include sequences from the Patp database, which is a proprietary database that contains sequences published in patents and patent publications. Patp results include those listed in Table 1F.

TABLE 1F
Patp alignments of NOV1

| | | | Smallest |
|---|---|---|---|
| | | | Sum |
| Sequences producing High-scoring | Reading | High | Prob. |
| Segment Pairs: | Frame | Score | P (N) |
| patp:AAW69215 Ostcoclast inhibitor | +2 | 2017 | 9.7e−208 |
| protein, OIP-2 [Homo sap], 433 aa | | | |
| patp:AAB36175 Human FDH02 protein, | +2 | 2017 | 9.7e−208 |
| [Homo sapiaens], 433 aa | | | |
| patp:AAY51114 Human cysteine protease | +2 | 1649 | 4.8e−185 |
| NCP protein [Homo sap], 433 aa | | | |
| patp:AAW15420 Human adrenal gland | | | |
| cysteine protease [Homo sap], 431 aa | | | |
| patp:AAW83925 Novel cysteine protease | | | |
| (NCP) polypeptide [Homo sap], 431 aa | | | |

Legumain is a cysteine endopeptidase that shows strict specificity for hydrolysis of asparaginyl bonds. The enzyme belongs to peptidase family C13, and is thus unrelated to the better known cysteine peptidases of the papain family, C1 (Rawlings and Barrett, Methods Enzymol 244, 461486, 1994). To date, legumain has been described only from plants and a blood fluke, Schistosoma mansoni. Legumain is also present in mammals. Legumain was cloned and sequenced from pig and later purified to homogeneity (2200-fold, 8% yield) from pig kidney. The mammalian sequences are clearly homologous with legumains from non-mammalian species. Pig legumain is a glycoprotein of about 34 kDa, decreasing to 31 kDa on deglycosylation. It is an asparaginyl endopeptidase, hydrolyzing Z-Ala-Ala-Asn-7-(4-methyl)coumarylamide and benzoyl-Asn-p-nitroanilide. Maximal activity is seen at pH 5.8 under normal assay conditions, and the enzyme is irreversibly denatured at pH 7 and above. Mammalian legumain is a cysteine endopeptidase, inhibited by iodoacetamide and maleimides, but unaffected by compound E64 (trans-epoxysuccinyl-L-leucylamido-(4-guanidino)butane). It is inhibited by ovocystatin (cystatin from chicken egg white) and human cystatin C with Ki values <5 nM. The discovery of a cysteine endopeptidase of a new family and distinctive specificity in man and other mammals may hold potential for future therapeutics. (Chen et al., J Biol Chem 272(12):8090-8, 1997; Choi et al., J Biol Chem 274(39):27747-53, 1999)

A clone that blocked both human and murine osteoclast (OCL) formation and bone resorption by more than 60%. This clone was identical to human legumain, a cysteine endopeptidase. Legumain significantly inhibited OCL-like multinucleated cell formation induced by 1,25-dihydroxyvitamin D(3)(1,25-(OH)(2)D(3)) and parathyroid hormone-related protein (PTHrP) in mouse and human bone marrow cultures, and one resorption in the fetal rat long bone assay in a dose-dependent manner. Legumain was detected in freshly isolated marrow plasma from normal donors and conditioned media from human marrow cultures. Furthermore, treatment of human marrow cultures with an antibody to legumain induced OCL formation to levels that were as high as those induced by 1,25-(OH)(2)D(3). Implantation in nude mice of 293 cells transfected with the legumain cDNA and constitutively expressing high levels of the protein significantly reduced hypercalcemia induced by PTHrP by about 50%, and significantly inhibited the increase in OCL surface and in OCL number expressed per mm(2) bone area and per mm bone surface induced by PTIrP. These results suggest that legumain may be a physiologic local regulator of OCL activity that can negatively modulate OCL formation and activity.

Site-directed mutagenesis has shown that the catalytic residues of mammalian legumain, a recently discovered lysosomal asparaginycysteine endopeptidase, form a catalytic dyad in the motif His-Gly-spacer-Ala-Cys. It is noted that the same motif is present in the caspases, aspartate-specific endopeptidases central to the process of apoptosis in animal cells, and also in the families of clostripain and gingipain which are arginyl/lysyl endopeptidases of pathogenic bacteria. (Chen et al., FEBS Lett 441(3):361-5, 1998)

Foreign protein antigens must be broken down within endosomes or lysosomes to generate suitable peptides that will form complexes with class II major histocompatibility complex molecules for presentation to T cells. However, it is not known which proteases are required for antigen processing. To investigate this, a domain of the microbial tetanus toxin antigen (TTCF) was exposed to disrupted lysosomes that had been purified from a human B-cell line. It has bee demonstrated that the dominant processing activity is not one of the known lysosomal cathepsins, which are generally believed to be the principal enzymes involved in antigen processing, but is instead an asparagine-specific cysteine endopeptidase. This enzyme seems similar or identical to a mammalian homologue of the legumain/haemoglobinase asparaginyl endopeptidasos found originally in plants and parasites. Competitive peptide inhibitors of B-cell asparaginyl endopeptidase (AEP) were designed that specifically block its proteolytic activity and inhibit processing of TTCF in vitro. In vivo, these inhibitors slow TTCF presentation to T cells, whereas preprocessing of TTCF with AEP accelerates its presentation, indicating that this enzyme performs a key step in TTCF processing. Also, it was shown that N-glycosylation of asparaine residues blocks AEP action in vitro. This indicates that N-glycosylation could eliminate sites of processing by AEP in mammalian proteins, allowing preferential processing of microbial antigens. (Manoury et al., Nature 396(6712):695-9, 1998)

The disclosed NOV1 nucleic acid of the invention encoding a asparaginyl endopeptidase-like protein includes the nucleic acid whose sequence is provided in Table 1, or a fragment thereof. The invention also includes a mutant or variant nucleic acid any of whose bases may be changed from the corresponding base shown in Table 1 while still encoding a protein that maintains its asparaginyl endopeptidase-like activities and physiological functions, or a fragment of such a nucleic acid. The invention further includes nucleic acids whose sequences are complementary to those just described, including nucleic acid fragments that are complementary to any of the nucleic acids just described The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 9% percent of the bases may be so changed.

The disclosed NOV1 protein of the invention includes the asparaginyl endopeptidase-like protein whose sequence is provided in Table 2. The invention also includes a mutant or variant protein any of whose residues may be changed from the corresponding residue shown in Table 2 while still encoding a protein that maintains its asparaginyl endopeptidase-like activities and physiological functions, or a functional fragment thereof. In the mutant or variant protein, up to about 14% percent of the residues may be so changed.

The invention further encompasses antibodies and antibody fragments, such as F_{ab} or (F_{ab})₂, that bind immunospecifically to any of the proteins of the invention.

The above defined information for this invention suggests that this asparaginyl endopeptidase-like protein (NOV1) may function as a member of a “Legumain family”. Therefore, the NOV1 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV1 nucleic acids and proteins of the invention are useful in potential therapeutic applications implicated in cancer including but not limited to bone cancer; bone disorders including but not limited to osteoporosis, osteopetrosis, arthritis, osteomyelitis, osteonecrosis, avascular necrosis, Paget's Disease; hematopoietic disorders, Spinal Diseases, immune disorders, regeneration (in vitro and in vivo), Endometriosis, Fertility, Diabetes, Autoimmune disease, Renal artery stenosis, Interstitial nephritis, Glomerulonephritis, Polycystic kidney disease, viral/bacterial/parasitic infections and/or other pathologies and disorders. For example, a cDNA encoding the asparaginyl endopeptidase-like protein (NOV1) may be useful in gene therapy, and the asparaginyl endopeptidase-like protein (NOV1) may be useful when administered to a subject in need thereof. By way of nonlimiting example, the compositions of the present invention will have efficacy for treatment of patients suffering from cancer including but not limited to bone cancer; bone disorders excluding but not limited to osteoporosis, osteopetrosis, arthritis, osteomyelitis, osteonecrosis, avascular necrosis, Paget's Disease; hematopoietic disorders, Spinal Diseases, immune disorders, regeneration (in vitro and in vivo), Endometriosis, Fertility, Diabetes, Autoimmune disease, Renal artery stenosis, Interstitial nephritis, Glomerulonephritis, Polycystic kidney disease, viral/bacterial/parasitic infections. The NOV1 nucleic acid encoding asparaginyl endopeptidase-like, protein, and the asparaginyl endopeptidase-like protein of the invention, or fragments thereof, may further be useful in diagnostic applications, wherein the presence or amount of the nucleic acid or the protein are to be assessed.

NOV1 nucleic acids and polypeptides are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 protein has multiple hydrophilic regions, each of which can be used as an immunogen. In one embodiment, a contemplated NOV1 epitope is from about amino acids 20 to 50. In another embodiment, a NOV1 epitope is from about amino acids 60 to 80. In additional embodiments, NOV1 epitopes are from about amino acids 125 to 145, from about amino acids 180 to 290 and from about amino acids 315 to 345. These novel proteins can be used in assay systems for functional analysis of various human disorders, which will help in understanding of pathology of the disease and development of new drug targets for various disorders.

NOV2

A disclosed NOV2 nucleic acid of 1262 nucleotides (also referred to as spec_{—}000-392) encoding a novel Tyrosyl-tRNA Synthetase-like protein is shown in Table 2A. An open reading frame was identified beginning with an ATG initiation codon at nucleotides 6-8 and ending with a TAA codon at nucleotides 1252-1254. A putative untranslated region upstream from the initiation codon and downstream from the termination codon is underlined in Table 2A, and the start and stop codons are in bold letters.

TABLE 2A
NOV2 Nucleotide Sequence

| | |
|---|---|
| AGTAAAATGAATATTATTGATGAATTAACTTGGCGCGACGCCATTAATCAGCAPACAAACGAAG | (SEQ ID NO:3) |
| AAAGACTTCGCGAGCTCGTTGAAGAAAAGAGCATTTCACTTTATTGCGGCGTTGATCCAACTGG | |
| TGATTCGATGCATATCCGACATTTGATTCCATTTATGATGATGAAAAGATTCCAATTGGCAGGC | |
| CATCATCCATATATTCTGATTGGCGGCGGAACAGGAACGATCGGCGACCCAAGCGGCCGCAAAA | |
| CAGAACGTGTGTTGCAAACGATGGAACAAGTGCAACATAACGTGGACGCACTTTCCAATCAAAT | |
| GAGAAAATTGTTTGGTAAAGATGCAAATATCACATTTGTGAACAACTACGACTGGTTGTCAAAA | |
| ATCTCTTTACTTGAATTTTTGAGAGACTACGGTAAAAACTTCAACATCAACACGATCTTAGCAA | |
| AAGATATCGTTGCCAGCCGTTTGGAAGTAGGAATCTCATTTACAGAGTTTACTTACCAAATCCT | |
| GCAATCTATCGACTTTTTGCATCTGCATAAAACATACGATGTGCAATTGCAAATCGGTGGAGCA | |
| GACCAATGGGGAAATATCACAGCTGGATTAGACCTAATCCGAAAATTAGAAGGACCAGAAGCAG | |
| AAGCGTTCGGTTTAACGATCCCATTGATGCTGAAGGCTGACGGAACGAAATTCGGAAAAACTGC | |
| AGGCGGTGCCGTTTGGCTTGATCCGAAGAAAACITCACCATTTGAGTTCTACCAATTCTGGTTG | |
| AATCAAGATGATCGCGATGTAGTGAAATACTTGAAATTCTTTACTTTCTTGTCTCAAGAAGAAA | |
| TCGAAGACTTAGCGAAAAAAGTAGAGACAGAACCTGAGAAACGTGAAGCACAACGCCGTTTGGC | |
| AGAAGAAGTTACAAGATTTGTGCATAGTGAAGAAGACTTGAAAGAAGCGCAAAAAATAACGCGC | |
| ACATTGTTCTCTGGAAATAATAAAGAACTAAATGCGGAAGAAATCCGCGCAGGATTCGGTAAAA | |
| TGCCGAACGTTGAAATTTCAAGCACACCTGAAAATATCGTGGAACTGCTTGTTTCCACAAAAAT | |
| TGAACCATCTAAACGTCAAGCACGTGAAGATGTTTCAAACGGAGCTATAAGTATTAACGGTGAC | |
| CGCGTTACCGATTTAAATTTTGTCATAAATCCATCCGATGAATTCGATGGTAAGTTTGTGGTTA | |
| TTCGAAAAGGTAAGAAAAATTACTTTTGGCCAAGTAATTGATTAG | |

The NOV2 nucleic acid was identified by TblastN using CuraGen Corporation's sequence file for Tyrosyl-tRNA Synthetase or homolog as run against the Genomic Daily Files made available by GenBank or from files downloaded from the individual sequencing centers. The nucleic acid sequence was predicted from the genomic file Genbank or Sequencing Center accession number: spec_{—}000 by homology to a known Tyrosyl-tRNA Synthetase or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The disclosed NOV2 nucleic acid sequence has 609 of 909 bases (66%) identical to a Bacillus subtilis Tyrosyl-tRNA Synthetase mRNA (GENBANK-ID: M77668)(E=2.3e⁻⁷⁸).

A NOV2 polypeptide (SEQ ID NO:4) encoded by SEQ ID NO:3 has 415 amino acid residues and is presented using the one-letter code in Table 2B. Signal P, Psort and/or Hydropathy results predict that NOV2 does not contain a signal peptide and is likely to be localized in the microbody (peroxisome) with a certainty of 0.5200. NOV2 has a molecular weight of 47267.3 Daltons.

TABLE 2B
Encoded NOV2 protein sequence.

| | |
|---|---|
| MNIIDELTWRDAINQQTNEERLRELVEEKSISLYCGVDPTGDSMHIGHLIPFMMMKRFQLAGHHP | (SEQ ID NO:4) |
| YILIGGGTGTIGDPSGRKTERVLQTMEQVQHNVDALSNQMRKLFGKDANITFVNNYDWLSKISLL | |
| EFLRDYGKNFNINTMLAKDIVASRLEVGISFTEFTYQILQSIDFLHLHKTYDVQLQIGGADQWGN | |
| ITAGLDLIRKLEGPEAEAFGLTIPLMLKADGTKFGKTAGGAVWLDPKKTSPFEFYQFWLNQDDRD | |
| VVKYLKFFTFLSQEEIEDLAKKVETEPEKREAGTRLAEEVTRFVHSEEDLKEAQKITRTLFSGNI | |
| KELNAEEIRAGFGKMPNVEISSTPENIVELLVSTKIEPSKRQAREDVSNGAISINGDRVTDLNFV | |
| INPSDEFDGKFVVIRKGKKNYFLAK | |

The disclosed NOV2 amino acid sequence has 242 of 416 amino acid residues (58%) identical to, and 324 of 416 residues (77%) positive with, the 422 amino acid residue TYROSYL-TRNA SYNTHETASE 1 (EC 6.1.1.1)(TYROSINE—TRNA LIGASE)(TYRRS 1) protein from Bacillus subtilis (ptnr:SPTREMBL-ACC: P22326)(E=1.2e⁻¹³²). The NOV2 amino acid sequence was also found to have 146 of 399 amino acid residues (36%) identical to, and 229 of 399 residues (57%) positive with, the 476 amino acid residue CGI-04 PROTEIN protein from Homo sapiens, (ptnr:SPTREMBL-ACC: Q9Y2Z4)(E=2.7e⁻⁶⁴). The global sequence homology (as defined by FASTA alignment with the fill length sequence of this protein) is 69% amino acid homology and 58% amino acid identity.

NOV2 also homology to the amino acid sequences shown in the BLASTP data listed in Table 2C.

TABLE 2C
BLAST results for NOV2

| Gene Index/ | Protein/ | Length | Identity | Positives | |
|---|---|---|---|---|---|
| Identifier | Organism | (aa) | (%) | (%) | Expect |
| gi\|135192\|sp\|P22326 | TYROSYL-TRNA | 422 | 241/416 | 322/416 | 1e−140 |
| \|SYY1_BACSU | SYNTHETASE 1 | | (57%) | (76%) | |
| | (TYROSINE— | | | | |
| | TRNA LIGASE) | | | | |
| | (TYRRS 1) | | | | |
| gi\|13701524\|dbj\| | tyrosyl-tRNA | 420 | 231/413 | 318/413 | 1e−140 |
| BAB42818.1\| (AP003134) | synthetase | | (55%) | (76%) | |
| | [Staphylococcus | | | | |
| | aureus | | | | |
| | subsp. | | | | |
| | aureus N315] | | | | |
| gi\|135197\|sp\|P00952 | TYROSYL-TRNA | 419 | 229/418 | 304/418 | 1e−132 |
| \|SYY_BACST | SYNTHETASE | | (54%) | (71%) | |
| | (TYROSINE— | | | | |
| | TRNA LIGASE) | | | | |
| | (TYRRS) | | | | |
| gi\|135196\|sp\|P04077 | TYROSYL-TRNA | 419 | 227/418 | 304/418 | 1e−131 |
| \|SYY_BACCA | SYNTHETASE | | (54%) | (72%) | |
| | (TYROSINE— | | | | |
| | TRNA LIGASE) | | | | |
| | (TYRRS) | | | | |
| gi\|14973611\|gb\| | tyrosyl-tRNA | 418 | 215/419 | 287/419 | 1e−116 |
| AAK76159.1\| (AE007499) | synthetase | | (50%) | (68%) | |
| | [Streptococcus | | | | |
| | pneumoniae] | | | | |

The homology of these sequences is shown graphically in the ClustalW analysis shown in Table 2D.

[image]

[image]

Tables 2E-G list the domain description from DOMAIN analysis results against NOV2. This indicates that the NOV2 sequence has properties similar to those of other proteins known to contain this domain.

TABLE 2E
Domain Analysis of NOV2

gnl|Pfam|pfam00579, tRNA-synt_1b, tRNA synthetases class I (W
and 31. (SEQ ID NO:32)
Length = 301 residues, 95.0% aligned
Score = 223 bits (567), Expect = 2e−59

| | | | |
|---|---|---|---|
| NOV2. | 27 | EEKSISLYCGVDPTGDSMHICHLIPFMMMKRFQLAGHHPYILIGGGTGTIGDPSGRKTER | 86 |
| | | +++ + +\| \| \|\|\|\| +\|+\|\|\|+\| \| + + \| \|\|\| + \|\| \|\|\|\|\| + \|\| | |
| Pfam00579: | 2 | KKRPLRVYTGPDPTGP-LMLGRLVPLMKLVQLQQAGHEVFFLIADLHALIGDPS-KSEER | 59 |
| NOV2: | 97 | VLQTMEQVQHNVDALSNQMRKLFGKDANITFVNNYDWLSKISLLEFLRDYGKNPNINTML | 146 |
| | | \| + + \| +\| \|\| \|\|\| + \| \|\|\| \|\|+\| +\| \|\| | |
| Pfam00579: | 60 | KLLSRTEEYANA------QLACGLDPEKVTIVNQSDWLEHLDLAWLLRDLGKHFTLNRML | 113 |
| NOV2: | 147 | AKDIVASRLEVG--ISFTEFTYQINQSIDFLHLHKTYDVQLQIGGADQWGNITAGLDLIR | 204 |
| | | \| \|\|+ \| \|\|\| \|\| \| +\|\|+ \| \| \| \| \|\|+\|\| \|+\| \| \|\| \| | |
| Pfam00579: | 114 | QFKTVKKRLKEGEGISFGEFLYPLLQAADILLLKAD----LVPGGSDQRGHIELGRDLAR | 169 |
| NOV2: | 205 | KLEGPEAEAFGLTIPLMLKADGT-KFGKTAGG-AVWLDPKKTSPPEFYQFWLNQDDRDVV | 262 |
| | | + + \|\|\| \|\|+ \|\| \| \|+ \|++\|\| ++ \| \|\|+\| | |
| Pfam00579: | 170 | RFNKKYKKPVGLTHPLLTGLDGGKKMSKSDPNSAIFLDDEPESVYKKIQKAYTDPDREVR | 229 |
| NOV2: | 263 | KYLRFFTFLSQEEIEDLAKKVETEPEKREAQRRLAEEVTRFVHSEEDLKEAQKITRTLFS | 322 |
| | | \| \|\| \|\| \|+ \|\|\|\| \|+\| +\|\|+ \|\|+ \|\| +\| + \| \| + \| | |
| pfam00579: | 230 | KLLKLFTELNPEEIERLSK--FLGDSPKEAEELLADYVTGLLHGGDLKKAAAEALNALLE | 287 |

TABLE 2F
Domain Analysis of NOV2

gnl|Smart|smart00363, S4, S4 RNA-binding domain (SEQ ID NO:33)
Length = 63 residues, 90.5% aligned
Score = 43.9 bits (102), Expect = 2e−05

| | | | |
|---|---|---|---|
| NOV2: | 355 | LLVSTKIEPSKRQAREDVSNGAISINGDRVTDLNFVINPSDEFDGKFVVIRKGKKNY | 411 |
| | | \|\| + \|\|+ \|\|\|+ + \| + +\|\| +\|\|\| ++++ \| \| + +++ \|\|\| | |
| smart00363: | 6 | LLARLGLAPSRSQARKLIEQGRVKNGKKVTDPSYIVKPGDVISVRGKELKRLKKNL | 62 |

TABLE 2G
Domain Analysis of NOV2

gnl|Pfam|pfam01479, S4, S4 domain. (SEQ ID NO:34)
Length = 48 residues, 87.5% aligned
Score = 36.2 bits (82), Expect = 0.004

| | | | |
|---|---|---|---|
| NOV2: | 355 | LLVSTKIEPSKRQAREDVSNGAISINGDRVTDLNFVINPSDE | 396 |
| | | +\| \|+ \|\|\|+ + +\| + +\|\| \|\| ++++ \| \|\| | |
| pfam01479: | 6 | ILARLGFASSPSQARQLIEHGHVKVNGKVVTIPSYIVKPGDE | 47 |

Patp BLAST results for NOV2 include those listed in Table 2H.

TABLE 2H
Patp alignments of NOV2

| | | | Smallest |
|---|---|---|---|
| | | | Sum |
| | Reading | High | Prob. |
| Sequences producing High-scoring | Reading | High | Prob. |
| Segment Pairs: | Frame | Score | P(N) |
| patp:AAW19781 Tyrosyl-tRNA synthetase | +2 | 1291 | 8.3e−131 |
| [Staphylococcus aureus], 420 aa | | | |

Aminoacyl-tRNA synthetases catalyze the aminoacylation of tRNA by their cognate amino acid. Because of their central role in linking amino acids with nucleotide triplets contained in tRNAS, aminoacyl-tRNA synthetases are thought to be among the first proteins that appeared in evolution. Kleeman et al. (1997) cloned cDNAs encoding tyrosyl-tRNA synthetase (YARS) from several different human cDNA libraries. The YARS cDNA sequence encodes a 528-amino acid polypeptide.(Kleeman et al., J Biol Chem 272:14420-5, 1997) Sequence analysis revealed that the carboxyl end of the protein contains a region with 49% identity to endothelial monocyte-activating polypeptide II (EMAP II). (Kao et al., J Biol Chein 267:20239-20247, 1992; Kao et al, J Biol Chem 269:25106-25119, 1994)

Cytokine-type activities are observed for the human tyrosyl-tRNA synthetase, largely considered as an essential enzyme for protein synthesis, only after cleavage into two fragments. These peptide fragments are novel elements in the orchestration of the tissue response to a cellular suicide program and should be viewed as highly differentiated adaptions of peptide modules with biological activity in more than one kind of environment.

While native human tyrosyl-tRNA synthetase (TyrRS) is inactive as a cell-signaling molecule, it can be split into two distinct cytokines. The enzyme is secreted under apoptotic conditions in culture where it is cleaved into an N-terminal fragment that harbors the catalytic site and into a C-domain fragment found only in the mammalian enzymes. The N-terminal fragment is an interleukin-8 (IL-8)-like cytokine (Baggiolini et al., J Clin Invest 84:1045-1049, 1989; Modi et al., Hum Genet 84:185-187, 1990) whereas the released C-domain is an endothelial-monocyte-activating polypeptide II (EMAP II)-like cytokine. Although the IL-8-like activity of the N-fragment depends on an ELR motif found in alpha-chemokines and conserved among mammalian TyrRSs, here it was shown that a similar (NYR) motif in the context of a lower eukaryote TyrRS does not confer the IL8-like activity. It was also shown that a heptapeptide from the C-domain has EMAP II-like chemotaxis activity for mononuclear phaocytes and polymorphonuclear leukocytes. Eukaryote proteins other than human TyrRS that have EMAP II-like domains have variants of the heptapeptide motif. Peptides based on these sequences are inactive as cytokines. Thus, the cytokine activities of split human TyrRS depend on highly differentiated motifs that are idiosyncratic to the mammalian system (Wakasugi and Schimmel, J Biol Chem 274:23155-9, 1999).

Aminoacyl-tRNA synthetases can be divided in two groups of equal size on the basis of differences in the structure of their active sites. The core of class I synthetases is the classical nucleotide-binding domain with its characteristic Rossmann fold. In contrast, the active site of class II synthetases is built around an antiparallel beta-sheet, to which the substrates bind. This classification, which is based on structural data (amino acid sequences and tertiary structures), can be rationalized in functional terms.

Human mini TyrRS differs in primary structure from more typical -chemokines. For example, while mini TyrRS contains an ELR motif that is critical for receptor binding, this motif is at the middle of the Rossmann fold that forms the site for synthesis of tyrosyl-adenylate. In contrast, the ELR motif of -chemokines is located near the N terminus. Also, whereas -chemokines have conserved cysteines and a Cys-Xaa-Cys motif (where Xaa is any residue), mini TyrRS does not share the conserved residues. Despite these differences in primary structures, human mini TyrRS is predicted (based on the crystal structure of Bacillus stearothermophilus TyrRS to form the same six-stranded -sheet as the -chemokines. Moreover, the predicted location of the ELR motif of human mini TyrRS is close to that of the -chemokines.

The above defined information for this invention suggests that this Tyrosyl-tRNA Synthetase-like protein (NOV2) may function as a member of a “Tyrosyl-tRNA Synthetase family”. Therefore, the NOV2 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV2 nucleic acids and proteins of the invention are useful in potential therapeutic applications implicated in antiviral and antitumor immune responses, inflammation and acute phase responses, as well as regulate cell proliferation, systemic juvenile rheumatoid arthritis, atherosclerosis, Multiple sclerosis, Osteopetrosis and/or other pathologies and disorders. For example, a cDNA encoding the Tyrosyl-tRNA Synthetase-like protein (NOV2) may be useful in gene therapy, and the Tyrosyl-tRNA Synthetase-like protein (NOV2) may be useful when administered to a subject in need thereof. By way of nonlimiting example, the compositions of the present invention will have efficacy for treatment of patients suffering from viral diseases and cancer, inflammatory diseases and acute phase responses, systemic juvenile rheumatoid arthritis, atherosclerosis, Multiple sclerosis, Osteopetrosis. The NOV2 nucleic acid encoding Tyrosyl-tRNA Synthetase-like protein, and the Tyrosyl-tRNA Synthetase-like protein of the invention, or fragments thereof, may further be useful in diagnostic applications, wherein the presence or amount of the nucleic acid or the protein are to be assessed.

NOV2 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen. In one embodiment, a contemplated NOV2 epitope is from about amino acids 10 to 40. In another embodiment, a NOV2 epitope is from about amino acids 60 to 120. In additional embodiments, NOV2 epitopes are from about amino acids 225 to 255, from about 260 to 340 and from about amino acids 350 to 380. These novel proteins can be used in assay systems for functional analysis of various human disorders, which are useful in understanding of pathology of the disease and development of new drug targets for various disorders.

NOV3

A disclosed NOV3 nucleic acid of 5730 nucleotides (also referred to as 32073570_EXT) encoding a novel Melastatin-like protein is shown in Table 3A. An open reading frame was identified beginning with an ATG initiation codon at nucleotides 136-138 and ending with a TAA codon at nucleotides 5728-5730. A putative untranslated region upstream from the initiation codon is underlined in Table 3A, and the start and stop codons are in bold letters.

TABLE 3A
NOV3 Nucleotide Sequence

| | |
|---|---|
| GAAGGTTTCCCTGGGCGTTCCTTGTCCGGCGGCCTCTGCTGCCGCCTCCGGAGACGCTTCCCG | (SEQ ID NO:5) |
| ATAGATGGCTACAGGCCGCGGAGGAGCAGGAGGTGGAGTTGCTGCCCTTCCGGAGTCCGCCCC | |
| GTGAGGAGAATGTCCCACAAATCCTGGATAGAAAGCACTTTGACCAAGAGGQAATGTGTATAT | |
| ATTATACCAAGTTCCAAGGACCCTCACAGATGCCTTCCAGGATGTCAAATTTGTCACCAACTC | |
| GTCAGGTGTTTTTGTGGTCGCTTGGTCAAGCAACATGCTTGTTTTACTGCAAGTCTTGCCATG | |
| AAATACTCAGATGTGAAATTGGGTGACCATTTTAATCACGCAATACAAGAATGGTCTGTGGAA | |
| AAGCATACAGAACAGAGCCCAACGGATGCTTATGGAGTCATAAATTTTCAAGGGGGTTCTCAT | |
| TCCTACAGAGCTAAGTATGTGAGGCTATCATATGACACCAAACCTGAAGTCATTCTGCAACTT | |
| CTGCTTAAAGAATGGCAAATGGAGTTACCCAAACTTGTTATCTCTGTACATGGGGGCATGCAG | |
| AAATTTGAGCTTCACCCACGAATCAAGCAGTTGCTTGGAAAAGGTCTTATTAAAGCTGCAGTT | |
| ACAACTGGAGCCTGGATTTTAACTGGAGGAGTAAACACAGGTGTGCCAPAACATGTTCGAGAT | |
| GCCCTCAAAGAACATGCTTCCAGATCATCTCGAAAGATTTGCACTATCOGAATAGCTCCATGG | |
| GCAGTGATTCAAAACAGAAATOATCTTGTTGGGAGAGATGTAGTTGCTCCTTATCAAACCTTA | |
| TTGAACCCCCTGAGCAAATTGAATGTTTTGAATAATCTGCATTCCCATTTCATATTGGTGGAT | |
| GATGGCACTGTTGGAAAGTATGGGGCGGAAGTCAGACTGAGAAGAGAACTTGAAAAAACTATT | |
| AATCAGCAAAGAATTCATGCTAGGATTGGCCAGGGTGTCCCTGTGGTGGCACTTATATTTGAG | |
| GGTGGGCCAAATGTTATCCTCACAGTTCTTGAATACCTTCACGAAAGCCCCCCTGTTCCAGTA | |
| GTTGTGTGTGAAGGAACAGGCAGAGCTGCAGATCTGCTAGCGTATATTCATAAACAAACAGAA | |
| GAAGCACGGAATCTTCCTCATGCACCAGAGCCCGATATTATTTCCACTATCAAAAAAACATTT | |
| AACTTTGGCCAGAATGAAGCACTTCATTTATTTCAAACACTGATGGAGTGCATGAAAAGAAAG | |
| GAGCTTATCACTGTTTTCCATATTGGGTCAGATGAACATCAAGATATAGATGTA9CAATACTT | |
| ACTGCACTGCTAAAAGGTACTAATGCATCTGCATTTGACCAGCTTATCCTTACATTGGCATGG | |
| GATACAGTTCACATTGCCAAAAATCATGTATTTGTTTATGGACAGCAGTGGCTGGTAGGATCC | |
| TTGGAACAAGCTATGCTTGATGCTCTTGTAATGGATAGAGTTGCATTTGTAAAACTTCTTATT | |
| GAAAATGGAGTAAGCATGCATAAATTCCTTACCATTCCGAGACTGGAAGAACTTTACAACACT | |
| AAACAAGGTCCAACTAATCCAATGCTGTTTCATCTTGTTCGAGACGTCAAACAGGGAAATCTT | |
| CCTCCACGATATAAGATCACTCTGATTGATATAGGACTTGTTATTGAATATCTCATGGGAGGA | |
| ACCTACAGATGCACCTATACTAGGAAACGTTTTCGATTAATATATAATAGTCTTGGTGGAAAT | |
| AATCGGAGGTCTGGCCGAAATACCTCCAGCAGCACTCCTCAGTTGCGAAAGAGTCATGAATCT | |
| TTTGGCAATAGGGCAGATAAAAAGGAAAAAATGAGGCATAACCATTTCATTAAGACAGCACAG | |
| CCCTACCGACCAAAGATTGATACAGTTATGGAAGAAGGAAAGAAGAAAAGAACCAAAGATGAA | |
| ATTGTAGACATTGATGATCCAGAAACCAAGCGCTTTCCTTATCCACTTAATGAACTTTTAATT | |
| TGGGCTTGCCTTATGAAGAGGCAGGTCATGGCCCGTTTTTTATGGCAACATGGTGAAGAATCA | |
| ATGGCTAAAGCATTAGTTGCCTGTAAGATCTATCGTTCAATGGCATATGAAGCAAAGCAGAGT | |
| GACCTGGTAGATGATACTTCACAAGAACTAAAACAGTATTCCAGTGATTTTGGTCAGTTGGCC | |
| GTTGAATTATTAGAACAGTCCTTCAGACAAGATGAAACCATGGCTATGAAATTGCTCACTTAT | |
| GAACTGAAGAACTGGAGTAATTCAACCTGCCTTAAGTTAGCAGTTTCTTCAAGACTTAGACCT | |
| TTTGTAGCTCACACCTGTACACAAATGTTGTTATCTGATATGTGGATGGGAAGGCTGAAAATG | |
| AGGAAAAATTCCTGGTACAAGGTAATACTAAGCATTTTAGTTCCACCTGCCATATTGCTGTTA | |
| GAGTATAAAACTAAGGCTGAAATGTCCCATATCCCACAATCTCAAGATGCTCATCAGATGACA | |
| ATGGATGACAGCGAAAACAACTTTCAGAACATAACAGAAGAGATCCCCATGGAAGTGTTTAAA | |
| GAAGTACGGATTTTGGATAGTAATGAAGGAAAGAATGAGATGGAGATACAAATGAAATCAAAA | |
| AAGCTTCCAATTACGCGAAAGTTTTATGCCTTTTATCATGCACCAATTGTAAAATTCTGGTTT | |
| AACACGTTGGCATATTTAGGATTTCTGATGCTTTATACATTTGTGGTTCTTGTACAAATGGAA | |
| CAGTTACCTTCAGTTCAAGAATGGATTGTTATTGCTTATATTTTTACTTATGCCATTGAGAAA | |
| GTCCGTGAGGTATTTATGTCTGAAGCTGGGAAAGTAAACCAGAAGATTAAAGTATGGTTTAGT | |
| GATTACTTCAACATCAGTGATACAATTGCCATAATTTCTTTCTTCATTGGATTTGGACTAAGA | |
| TTTGGAGCAAAATGGAACTTTGCAAATGCATATGATAATCATGTTTTTGTGGCTGGAAGATTA | |
| ATTTACTGTCTTAACATAATATTTTGGTATGTGCGTTTGCTAGATTTTCTAGCTGTAAATCAA | |
| CACGCAGGACCTTATGTAATGATGATTCGAAAAATGGTGGCCAATATGTTCTACATTGTAGTG | |
| ATTATGGCTCTTGTATTACTTAGTTTTGGTGTTCCCAGAAAGGCAATACTTTATCCTCATGAA | |
| GCACCATCTTGGACTCTTGCTAAAGATATAGTTTTTCACCCATACTGGATGATTTTTCGTGAA | |
| GTTTATGCATACCAAATTCATGTGTGTGCAAATGATTCTGTTATCCCTCAAATCTGTGGTCCT | |
| GGGACGTGGTTGACTCCATTTCTTCAAGCAGTCTACCTCTTTGTACAGTATATCATTATGGTT | |
| AATCTTCTTATTGCATTTTTCAGCAATGTGTATTTACAAGTGAAGGCAATTTCCAATATTGTA | |
| TGGAAGTACCAGCGTTATCATTTTATTATGGCTTATCATGAGAAACCAGTTCTGCCTCCTCCA | |
| CTTATCATTCTTAGCCATATAGTTTCTCTGTTTTGCTGCATATGTAAGAGAAGAAAGAAAGAT | |
| AAGACTTCCGATGOACCAGAACTTTTCTTAACAGAAGAAGATCAAAAGAAACTTCATGATTTT | |
| GAAGAGCAGTGTCTTGAAATGTATTTCAATGAAAAAGATGACAAATTTCATTCTGGGACTGAA | |
| GAGAGAATTCGTGTCACTTTTGAAAGCGTGGAACAGATGTGCATTCAGATTAAAGAAGTTGCA | |
| GATCGTGTCAACTACATAAAAAGATCATTACAATCATTAGATTCTCAAATTGGCCATTTGCAA | |
| GATCTTTCAOCCCTGACGGTAGATACATTAAAAACACTCACTGCCCAGAAAGCGTCGGAAGCT | |
| AGCAAAGTTCATAATGAAATCACACGAGAACTCAGCATTTCCAAACACTTGGCTCAAAACCTT | |
| ATTGATGATGGTCCTGTAAGACCTTCTGTATGGAAAAAGCATGGTGTTGTAAATACACTTAGC | |
| TCCTCTCTTCCTCAAGGTGATCTTGAAAGTAATAATCCTTTTCATTGTAATATTTTAATGAAA | |
| GATGACAAAGATCCCCAGTGTAATATATTTGGTCAAGACTTACCTGCAGTACCCCAGAGAAAA | |
| GAATTTAATTTTCCAGAGGCTGGTTCCTCTTCTGGTGCCTTATTCCCAAGTGCTGTTTCCCCT | |
| CCAGAACTGCGACAGAGACTACATGGGGTAGAACTCTTAAAAATATTTAATAAAAATCAAAAA | |
| TTAGGCAGTTCATCTACTAGCATACCACATCTGTCATCCCCACCAACCAAATTTTTTGTTAGT | |
| ACACCATCTCAGCCAAGTTGCAAAAGCCACTTGGAAACTGGAACCAAACATCAAGAAACTGTT | |
| TGCTCTAAACCTACAGAAGGAGATAATACAGAATTTGGAGCATTTGTAGGTCACACAGATAGC | |
| ATGGATTTACAGAGGTTTAAAGAAACATCAAACAAGATAAAAATACTATCCAACAATACTTCT | |
| GAAAACACTTTGAAACGAGTGAGTTCTCTTGCTGGATTTATTGACTGTCACAGAACTTCCATT | |
| CCTGTTCATTCAAAACAAGCAGAAAAAATCAGTACAAGGCCATCTACCGAAGACACTCATGAA | |
| GTAGATTCCAAAGCAGCTTTACTGAAGGATTCGTTACAAGATAOACCATCAAACAGAGAAATG | |
| CCATCTGAAGAAGGAACATTAAATGGTCTCACTTCTCCATTTAAGCCAGCTATGGATACAAAT | |
| TACTATTATTCAGCTGTGGAAAGAAATAACTTGATGAGGTTATCACAGAGCATTCCATTTACA | |
| CCTGTGCCTCCAAGAGGTGAGCCTCTCACAGTGTATCGTTTGGAAGAGAGTTCACCCAACATA | |
| CTAAATAACAGCATGTCTTCTTGGTCACAACTAGGCCTCTGTGCCAAAATAGAGTTTTTAAGC | |
| AAAGAGGAGATGGGAGGAGGTTTACGAAGAGCTGTCAAAGTACAGTGTACCTGGTCAGAACAT | |
| GATATCCTCAAATCAGGGCATCTTTATATTATCAAATCTTTTCTTCCAGAGGTGGTTAATACA | |
| TGGTCAACTATTTACAAAGAAGATACAGTTCTGCATCTCTGTCTGAGAGAAATTCAACAACAG | |
| AGAGCAGCACAAAAGCTTACGTTTGCCTTTAATCAAATGAAACCCAAATCCATACCATATTCT | |
| CCAAGGTTCCTTGAAGTTTTCCTGCTGTATTGCCATTCAGCAGGACAGTGGTTTGCTGTGGAA | |
| GAATGTATGACTGGAGAATTTAGAAAATACAACAATAATAATGGAGATGAGATTATTCCAACT | |
| AATACTCTGGAAGAGATCATGCTAGCCTTTAGCCACTGGACTTACGAATAThCAAGAGGGGAG | |
| TTACTGGTACTTGATTTGCAAGGTGTTGGTGAAAATTTGACTGACCCATCTGTGATAAAAGCA | |
| GAAGAAAAGAGGTCCTGTGATATGGTTTTTGGCCCAGCAAATCTAGGACAAGATGCAATTAAA | |
| AACTTCAGAGCAAAACATCACTGTAATTCTTGCTGTAGAAAOCTTAAACTTCCAGATCTGAAG | |
| AGGAATGATTATACGCCTGATAAAATTATATTTCCTCAGGATGAGCCTTCAGATTTGAATCTT | |
| CAGCCTGGAAATTCCACCAAAGAATCAOAATCAACTAATTCTGTTCGTCTGATGTTATAA | |

The disclosed NOV3 nucleic acid sequence of this invention has 5189 of 5729 bases (90%) identical to a transient receptor potential-related protein (ChaK) mRNA from Mus musculus (GENBANK-ID:AF149013|acc:AF149013)(E=0.0).

A NOV3 polypeptide (SEQ ID NO:6) encoded by SEQ ID NO:5 is 1864 amino acid residues and is presented using the one-letter code in Table 3B. Signal P, Psort and/or Hydropathy results predict that NOV3 does not contain a signal peptide and is likely to be localized to the plasma membrane with a certainty of 0.6000. This is predicted as NOV3 is similar to the Transient receptor potential-related protein family, some members of which are presented at the plasma membrane.

TABLE 3B
Encoded NOV3 protein sequence.

| | |
|---|---|
| MSQKSWIESTLTKRECVYIIPSSKDPHRCLPGCQICQQLVRCFCGRLVKQRACFTASLAMKYSD | (SEQ ID NO:6) |
| VKLGDHFNQAIEEWSVEKMTEQSPTDAYGVINFQGGSHSYRAKYVRLSYDTKPEVILQLLLKEW | |
| QMELPKLVISVHGGMQKFELHPRIKQLLGKGLIKAAVTTGAWILTGGVNTGVAKHVGDALKEHA | |
| SRSSRKICTIGIAPWGVIENRNDLVGRDVVAPYQTLLNPLSKLNVLNNLHSHFILVDDGTVGKY | |
| GAEVRLRRELEKTINQQRIHARIGQGVPVVALIFEGGPNVILTVLEYLQESPPVPVVVCEGTGR | |
| AADLLAYIHKQTEEGGNLPDAAEPDIISTIKKTFNFGQNEALHLFQTLMECMKRKELITVFHIG | |
| SDEHQDIDVAILTALIKGTNASAFDQLILTLAWDRVDIAKNHVFVYGQQWLVGSLEQAMLDALV | |
| MDRVAFVKLLIENGVSMHKFLTIPRLEELYNTKQGPTNPMLFHLVRDVKQGNLPPGYKITLIDI | |
| GLVIEYLMGGTYRCTYTRKRFRLIYNSLGGNNRRSGRNTSSSTPQLRKSHESFGNRADKKEKMR | |
| HNHFIKTAQPYRPKIDTVMEEGKKKRTKDEIVDIDDPETKRFPYPLNELLIWACLMKRQVMARF | |
| LWQHGEESMAKALVACKIYRSMAYEAKQSDLVDDTSEELKQYSSDFGQLAVELLEQSFRQDETM | |
| AMKLLTYELKNWSNSTCLKLAVSSRLRPFVAHTCTQMLLSDMWMGRLNMRKNSWYKVILSILVP | |
| PAILLLEYKTKAEMSHIPQSQDAHQMTMDDSENNFQNITEEIPMEVFKEVRILDSNEGKNEMEI | |
| QMKSKKLPITRKFYAFYHAPIVKFWFNTLAYLGFLMLYTFVVLVQMEQLPSVQEWIVIAYIFTY | |
| AIEKVREVFMSEAGKVNQKIKVWFSDYFNISDTIAIISFFIGFGLRFGAKWNFANAYDNHVFVA | |
| GRLIYCLNIIFWYVRLLDFLAVNQQAGPYVMMIGKMVANMFYIVVIMALVLLSFGVPRKAILYP | |
| HEAPSWTLAKDIVFHPYWMIFGEVYAYEIDVCANDSVIPQICGPGTWLTPFLQAVYLFVQYIIM | |
| VNLLIAFFSNVYLQVKAISNIVWKYQRYHFIMAYHEKPVLPPPLIILSHIVSLFCCICKRRKKD | |
| KTSDGPELFLTEEDQKKLHDFEEQCVEMYFNERDDKFHSGSEERIRVTFERVEQMCIQIKEVGD | |
| RVNYIKRSLQSLDSQIGHLQDLSALTVDTLKTLTAQKASEASKVHNEITRELSISKHLAQNLID | |
| DGPVRPSVWKKEGVVNTLSSSLPQCDLESNNPFHCNILMKDDKDPQCNIFCQDLPAVPQRKEFN | |
| FPEAGSSSGALFPSAVSPPELRQRLHGVELLKIFNKNQKLGSSSTSIPHLSSPPTKFFVSTPSQ | |
| PSCKSHLETGTKDQETVCSKATECDNTEFCAFVCHRDSMDLQRFKETSNKIKILSNNTSENTLK | |
| RVSSLACFIDCHRTSIPVHSKQAEKISRRPSTEDTHEVDSKAALLKDWLQDRPSNREMPSEEGT | |
| LNGLTSPFKPAMDTNYYYSAVERNNLMRLSQSIPFTPVPPRGEPVTVYRLEESSPNILNNSMSS | |
| WSQLGLCAKIEFLSKEEMGGGLRRAVKVQCTWSEHDILKSGHLYIIKSELPEVVNTWSSIYKED | |
| TVLHLCLREIQQQRAAQKLTFAFNQMKPKSIPYSPRFLEVFLLYCHSAGQWFAVEECMTGEFRK | |
| YNNNNGDEIIPTNTLEEIMLAFSHWTYEYTRGELLVLDLQGVGENLTDPSVIKAEEKRSCDMVF | |
| GPANLGEDAIKNFRAKHHCNSCCRKLKLPDLKRNDYTPDKIIFPQDEPSDLNLQPGNSTKESES | |
| TNSVRLML | |

The disclosed NOV3 amino acid sequence has 1756 of 1864 amino acid residues (94%) identical to, and 1811 of 1864 amino acid residues (97%) similar to, the 1863 amino acid residue Transient receptor potential-related protein from Mus musculus (TREMHLNEW-ACC:AAF73131)(E 0.0).

NOV3 maps to chromosome 15 and is expressed in at least the following tissues: fetal lung, lymph, prostate, colon, and carcinoma cell lines. In addition, the sequence is predicted to be expressed in the following tissues based on the expression pattern of its homolog, (GENBANK-ID:AF149013|acc:AF149013 Mus musculus transient receptor potential-related protein (ChaK) mRNA): embryonic tissue.

NOV3 also has homology to the amino acid sequences shown in the BLASTP data listed in Table 3C.

TABLE 3C
BLAST results for NOV3

| Gene Index/ | Protein/ | Length | Identity | Positives | |
|---|---|---|---|---|---|
| Identifier | Organism | (aa) | (%) | (%) | Expect |
| gi\|7020006\|dbj\| | testicular | 575 | 573/574 | 574/574 | 0.0 |
| BAA90958.1\| | Metalloprotease- | | (99%) | (99%) | |
| (AK000124) | like, | | | | |
| unnamed protein | Disintegrin- | | | | |
| product [Homo | like, Cysteine- | | | | |
| sapiens] | rich protein | | | | |
| | IVa [Macaca | | | | |
| | fascicularis] | | | | |
| gi\|14736335\|ref\| | testicular | 955 | 599/980 | 736/980 | 0.0 |
| Xp_028830.1\|61706 | Metalloprotease- | | (61%) | (74%) | |
| [Homo sapiens] | like, | | | | |
| | Disintegrin- | | | | |
| | like, Cysteine- | | | | |
| | rich protein | | | | |
| | IVb [Macaca | | | | |
| | fascicularis] | | | | |
| gi\|9929957\|dbj\| | cellular | 640 | 599/651 | 605/651 | 0.0 |
| BAB12135.1\| | disintegrin | | (92%) | (92%) | |
| (AB047611) | ADAM 6d; | | | | |
| hypothetical | tMDCIVd | | | | |
| protein [Macaca | [Oryctolagus | | | | |
| fascicularis] | cuniculus] | | | | |
| gi\|10946830\|ref\| | cellular | 1863 | 1756/1865 | 1811/1865 | 0.0 |
| Np_067425.1\| RIKEN | disintegrin | | (94%) | (96%) | |
| cDNA 5033407022 | ADAM 6e; | | | | |
| [Mus musculus] | tMDCIVe | | | | |
| | [Oryctolagus | | | | |
| | cuniculus] | | | | |
| gi\|14009344\|gb\| | tMDC IV [Rattus | 1863 | 1757/1865 | 1811/1865 | 0.0 |
| AAK50377.1\| | norvegicus] | | (94%) | (96%) | |
| (AY032951) LTRPC7 | | | | | |
| [Mus musculus] | | | | | |

The homology of these sequences is shown graphically in the ClustalW analysis shown in Table 3D.

[image]

[image]

[image]

[image]

[image]

[image]

[image]

[image]

Tables 3E and 3F list the domain description from DOMAIN analysis results against NOV3. This indicates that the NOV3 sequence has properties similar to those of other proteins known to contain this domain.

TABLE 3E
Domain Analysis of NOV3

gnl|Pfam|pfam02816, MHCK_EF2_kinase, MHCK/EF2 kinase domain
family (SEQ ID NO:40)
Length = 206 residues, 94.7% aligned
Score = 81.6 bits (200), Expect = 3e−16

| | | | |
|---|---|---|---|
| NOV3: | 1601 | WSQLGLCAKIEFLSKEEMGGGLRRVKVQCTWSEHDILKSGHLYIIKSFLPEVVNTWSSI | 1660 |
| | | \|+ + \|+\| \|+ \| +\| \| + \|+ \| \| \| + | |
| Pfam02861: | 12 | WTDDEVLVKVE--SQPFAEGAMREAYHTK------DLSNFLHAQQWKG----VGKYVAYR | 59 |
| NOV3: | 1661 | YKEDTVLHLCLREIQQRAAQKLTFAFNQMKP-KSIPYSPRFLEVFLLYCHSAGQW-FAV | 1718 |
| | | \| + \| +++ \| \|+\| +\|+ \|\| \| \| + \| \| + | |
| Pfam02861: | 60 | YIKPTDRDSYFEDVKMQMEAKKWGEKYNRHKPPKKIEFLQSC--VIELIDRPPSYPLCGL | 117 |
| NOV3: | 1719 | EECMTGEFRKYNNNNGDEIIPTNTLEEIMLAFSHWTYEYTRGELLVLDLQGVGENLTDPS | 1778 |
| | | \| + \|+++\|\|\|+\|+\| ++ + \|\|\|\|+\|\|\| + +\|+\|+\|+\|\|\|\|+ \|\|\| | |
| Pfam02861: | 118 | EPYIEGKYKKYNSNSG---FVSDNIRNTPQAPSHFTYELSNHQLIVVDIQGVGDLYTDPQ | 174 |
| NOV3: | 1779 | VIKAEEKRSCDMVFGPANLGEDAIKNFRAKHHCNSCC | 1815 |
| | | \| \|+ \|\| \|\|\| \| \| \|\|+ \| | |
| Pfam02861: | 175 | -IHTEDGTG----FGDGNLGVRGFAKFLYTHKCNAIC | 206 |

TABLE 3F
Domain Analysis of NOV3

gnl|Pfam|pfam00520, ion_trans, Ion transport protein. (SEQ ID NO:41)
Length = 188 residues, 97.9% aligned
Score = 62.0 bits (149), Expect = 3e−10

| | | | |
|---|---|---|---|
| NOV3: | 886 | EWIVIAYIFTYAIEKVREVFMSEAGKVNQKIKVWFSDYFNISDTIAIISFFIGFGLRFGA | 945 |
| | | \| + + + +\| + + + +\|\| +\|−− \| \| | |
| Pfamn00520: | 3 | EILDYVFTVIFTLEILLKFIALGF------KAKYLRSPWNILDFLAVLPSLIDLILFLLG | 56 |
| NOV3: | 946 | KWNFANAYDNHVFVAGRLIYCLNIIFWYVRLLDFLAVNQQAGPYVMMIGKMVANMFYIVV | 1005 |
| | | + +\|\|\| \| + + +\|+ + ++ +++ | |
| Pfam00520: | 57 | GGRVLR------------------LLRLLRLLRLLRRLETLRTLLQSLGRSLKSLLNLLL | 98 |
| NOV3: | 1006 | IMALVLLSFGVPRKAILPHEAPSWTLAKDIVFHPYWMIFGEVYAYEIDVCANDSVIPQI | 1065 |
| | | ++ \|+\| \| + \|+ \| \| + + + + + + + | |
| Pfam00520: | 99 | LLLLLLFIFAI-GVQLFGGEKYLCCDINPINGNSNFDSYFDAFYWLFRTLTTVGWGDIM | 157 |
| NOV3: | 1066 | CGPGTWLTPFLQAVYLFVQYIIMVNLLIA | 1094 |
| | | \|\| +++ + ++++\|\|\|\|\| | |
| Pfam00520: | 158 | PDTLDWLGKIFFVIFIILGGLLLLNLLIA | 186 |

Patp BLAST results for NOV3 include those listed in Table 3G.

TABLE 3G
patp alignments of NOV3

| | | | Smallest |
|---|---|---|---|
| | | | Sum |
| Sequences producing High-scoring | Reading | High | Prob. |
| Segment Pairs: | Frame | Score | P (N) |
| patp:AAY95435 Human calcium channel | +1 | 9751 | 0.0 |
| SOC-2/CRAC-1 [Homo sap], 1865 aa | | | |
| patp:AAY95433 Hum SOC-2/CRAC-1 | +1 | 6387 | 0.0 |
| C-term polypeptide [Home sap], 1224 aa | | | |

Melastatin belongs to the transient receptor potential protein (Trp) family of calcium channels. Members of the TRP family may play a role in calcium homeostasis. The Drosophila trp (transient retinal potential) and trpl (trp-like) genes encode plasma membrane cation channels that, as Zhu et al. state, may allow calcium influx in non-excitable cells in response to depletion of intracellular calcium pools, a process referred to as capacitative calcium entry (CCE), as part of the phototransduction process. (Zhu et al., Cell 85(5):661-71, 1996) Melastatin expression is reported to be inversely correlated with melanoma aggressiveness. Many studies describe the cloning and function of the known members of the trp family of genes.

A differential cDNA display was used to search for genes whose expression correlates with an aggressive phenotype in variants of the B16 murine melanoma line, B16-F1 and B16-F 10. This analysis identified a novel gene, termed melastatin, that is expressed at high levels in poorly metastatic variants of B 16 melanoma and at much reduced levels in highly metastatic B 16 variants. Melastatin was also found to be differentially expressed in tissue sections of human melanocytic neoplasms. Benign nevi express high levels of melastatin, whereas primary melanomas showed variable melastatin expression. Melastatin transcripts were not detected in melanoma metastases. Within the set of human primary cutaneous melanomas examined, melastatin expression appeared to correlate inversely with tumor thickness. The expression pattern observed suggests that loss of melastatin expression is an indicator of melanoma aggressiveness. (Duncan et al., Cancer Res 58(7):1515-20, 1998)

A novel gene, melastatin, was recently described whose expression is inversely correlated with melanoma aggressiveness. Chromosomal localization of this gene places it on mouse chromosome 7 and in the 15q13-q14 region of the human genome. Although expression patterns and chromosomal localization in the mouse are consistent with involvement of melastatin mutations in the mouse ruby-eye-2 defect, congenic analysis showed genetic segregation of the two loci. Cloning of the full-length human cDNA revealed a much larger transcript than we had previously identified, corresponding to a 1533-amino-acid protein product with homology to members of the transient receptor potential (Trp) family of calcium channels. The mouse melastatin gene contains 27 exons and spans at least 58 kb of genomic DNA. The promoter region of Mlsn1 contains four potential microphthalmia binding sites including an M box, a transcriptional regulatory element unique to genes with a restricted melanocytic expression pattern. A 1-kb PvuII fragment from this region was capable of driving high levels of luciferase expression in B 16 melanoma cells. (Hunter et al., Genomics 54(1):116-23, 1998)

A novel putative Ca(2+) channel gene, MTR1, was recently described which shows a high level of homology to the human TRPC7 gene and the melastatin 1 (MLSN1) gene, another Trp (transient receptor potential protein)-related gene whose transcript was found to be downregulated in metastatic melanomas. It maps to human chromosome band 11p15.5, which is associated with the Beckwith-Wiedemann syndrome and predisposition to a variety of neoplasias. The isolation and characterization of the murine orthologue Mtr1 was also reported. The chromosomal localization on distal chromosome 7 places it in a cluster of imprinted genes, flanked by the previously described Tapa1 and Kcnq1 genes. The Mtr1 gene encodes a 4.4-kb transcript, present in a variety of fetal and adult tissues. The putative open reading frame consists of 24 exons, encoding 1158 amino acids. Transmembrane prediction algorithms indicate the presence of six membrane-spanning domains in the proposed protein. Imprinting analysis, using RT-PCR on RNA from reciprocal mouse crosses harboring a −20 sequence polymorphism, revealed biallelic expression of Mtr1 transcripts at all stages and tissues examined. (Enklaar et al., Genomics 67(2):179-87, 2000)

Alterations within human chromosomal region 11p15.5 are associated with the Beckwith-Wiedemann syndrome (BWS) and predisposition to a variety of neoplasias, including Wilms' tumors (WTs), rhabdoid tumors and rhabdomyosarcomas. To identify candidate genes for 11p15. 5-related diseases, human genomic sequence were compared with expressed sequence tag and protein databases from different organisms to discover evolutionarily conserved sequences. The identification and characterization of a novel human transcript related to a putative Caenorhabditis elegans protein and the trp (transient receptor potential) gene was described. The highest homologies are observed with the human TRPC7 and with melastatin 1 (MLSN1), whose transcript is downregulated in metastatic melanomas. Other genes related to and interacting with the trp family include the Grc gene, which codes for a growth factor-regulated channel protein, and PKD1/PKD2, involved in polycystic kidney disease. The novel gene (named MTR1 for MLSN1- and TRP-related gene 1) resides between TSSC4 and KvLQT1. MTR1 is expressed as a 4.5 kb transcript in a variety of fetal and adult tissues. The putative open reading frame is encoded in 24 exons, one of which is alternatively spliced leading to two possible proteins of 872 or 1165 amino acids with several predicted membrane-spanning domains in both versions. MTR1 transcripts arc present in a large proportion of WTs and rhabdomyosarcomas. RT-PCR analysis of somatic cell hybrids harboring a single human chromosome 11 demonstrated exclusive expression of MTR1 in cell lines carrying a paternal chromosome 11, indicating allele-specific inactivation of the maternal copy by genomic imprinting. (Prawitt et al., Hum Mol Genet 9(2):203-16, 2000)

The protein similarity information, expression pattern, and map location for the Melastatin-like protein and nucleic acid (NOV3) disclosed herein suggest that this Melastatin-like protein may have important structural and/or physiological functions characteristic of the transient receptor potential-related protein family. Therefore, the NOV3 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV3 nucleic acids and proteins of the invention are useful in diagnostic and therapeutic applications implicated in various diseases and disorders described below and/or other pathologies. For example, the compositions of the present invention will have efficacy for treatment of patients suffering from systemic lupus erythematosus, autoimmune disease, asthma, emphysema, scleroderma, allergy, ARDS, Hirschsprung's disease, Crohn's disease, appendicitis, inflammatory bowel disease, diverticular disease, melanoma, Wilm's tumor, rhabdomyosarcomas cancer, hemophilia, hypercoagulation, carciovascular disorder's, restenosis, idiopathic thrombocytopenic purpura, allergies, immunodeficiencies, transplantation, graft versus host disease (GVHD), lymphaedema, fertility disorders, growth disorders, regulatory disorders, and developmental disorders. The NOV3 novel nucleic acids, or fragments thereof, may further be useful in diagnostic applications, wherein the presence or amount of the nucleic acid or the protein are to be assessed.

NOV3 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV3 protein has multiple hydrophilic regions, each of which can be used as an immunogen. These NOV3 proteins can be used in assay systems for functional analysis of various human disorders, which will help in understanding of pathology of the disease and development of new drug targets for various disorders.

NOV4

A disclosed NOV4 nucleic acid of 1811 nucleotides (also referred to as 124141642_EXT) encoding a novel leucine-rich repeat proteins-like protein is shown in Table 4A. An open reading frame was identified beginning with an ATG initiation codon at nucleotides 17-19 and ending with a TGA codon at nucleotides 1793-1795. A putative untranslated region upstream from the initiation codon and downstream from the termination codon is underlined in Table 4A, and the start and stop codons are in bold letters.

TABLE 4A
NOV4 Nucleotide Sequence

| | |
|---|---|
| GCCCACGCTCCGCACCATGACCTGCTGGCTGTGCGTCCTGAGCCTGCCCCTGCTCCTGCTGCC | (SEQ ID NO:7) |
| CGCGGCGCCGCCCCCGGCTGGAGGCTGCCCGGCCCGCTGCGAGTGCACCGTGCAGACCCGCGC | |
| GGTGGCCTGCACGCGCCGCCGCCTQACCGCCGTGCCCGACGGCATCCCGGCCGAGACCCGCCT | |
| GCTGGAGCTCAGCCGCAACCGCATCCGCTGCCTGAACCCGGGCGACCTGGCCGCGCTGCCCGC | |
| GCTGGAGGAGCTGGACCTGAGCGAGAACGCCATCGCGCACGTGGAGCCCGGCGCCTTCGCCAA | |
| CCTGCCGCGCCTGCGCGTCCTGCGTCTCCGTGGCAACCAGCTGAAGCTCATCCCGCCCGGGGT | |
| CTTCACGCGCCTGGACAACCTCACGCTGCTGGACCTGAGCGAGAACAAGCTGGTAATCCTGCT | |
| GGACTACACTTTCCAGGACCTGCACAGCCTGCGCCGGCTGGAAGTGGGCGACAACGACCTGGT | |
| ATTCGTCTCGCGCCGCGCCTTCGCGGGGCTGCTGGCCCTGGAGGAGCTGACCCTGGAGCGCTG | |
| CAACCTCACGGCTCTGTCCGGGGAGTCGCTGGGCCATCTGCGCAGCCTGGGCGCCCTGCGGCT | |
| GCGCCACCTGGCCATCGCCTCCCTGGAGGACCAGAACTTCCGCAGGCTGCCCGGGCTGCTGCA | |
| CCTGGAGATTGACAACTGGCCGCTGCTGGAGGAGGTGGCGGCGGGCAGCCTGCGGGGCCTGAA | |
| CCTGACCTCGCTGTCGGTCACCCACACCAACATCACCGCCGTGCCGGCCGCCGCGCTGCGGCA | |
| CCAGGCGCACCTCACCTGCCTCAATCTGTCGCACAACCCCATCAGCACGGTGCCGCGGGGGTC | |
| GTTCCGGGACCTGGTCCGCCTGCGCGAGCTGCACCTGGCCGGGGCCCTGCTGGCTGTGGTGGA | |
| GCCGCAGGCCTTCCTGGGCCTGCGCCAGATCCGCCTGCTCAACCTCTCCAACAACCTGCTCTC | |
| CACGTTGGAGGAGAGCACCTTCCACTCGGTGAACACGCTAGAGACGCTGCGCGTGGACGGGAA | |
| CCCGCTGGCCTGCGACTGTCGCCTGCTGTGGATCGTGCAGCGTCGCAAGACCCTCAACTTCGA | |
| CGGGCGGCTGCCGGCCTGCGCCACCCCGGCCGAGGTGCGCGGCGACGCGCTGCGAAACCTGCC | |
| GGACTCCGTGCTGTTCGAGTACTTCGTGTGCCGCAAACCCAAGATCCGGGAGCGGCGGCTGCA | |
| GCGCGTCACGGCCACCGCGGGCGAAGACGTCCGCTTCCTCTGCCGCGCCGAGGGCGAGCCGGC | |
| GCCCACCGTGGCCTGGGTGACCCCCCAGCACCGGCCGGTGACGGCCACCAGCGCGGGCCGGGC | |
| GCGCGTGCTCCCCGGGGGGACGCTGGAGATCCAGGACGCGCGGCCGCAGGACAGCGGCACCTA | |
| CACGTGCGTGGCCAGCAACGCGGGCGGCAACGACACCTACTTCGCCACGCTGACCGTGCGCCC | |
| CGAGCCGGCCGCCAACCGGACCCCGGGCGAGGCCCACAACGAGACGCTGGCGGCCCTGCGCGC | |
| GCCGCTCGACCTCACCACCATCCTGGTGTCCACCGCCATGGGCTGCATCACCTTCCTGGGCGT | |
| GGTCCTCTTCTGCTTCGTGCTGCTGTTCGTGTGGAGCCGCGGCCGCGGGCAGCACAAAAACAA | |
| CTTCTCGGTGGAGTACTCCTTCCGCAAGGTGGATGGGCCGGCCGCCGCGGCGGGCCAGGGAGG | |
| CGCGCGCAAGTTCAACATGAAGATGATCTGAGGGGTCCCCAGGGCGG | |

The disclosed NOV4 nucleic acid sequence has 963 of 1612 bases (59%) identical to a human IGF binding protein complex acid-labile subunit a mRNA (GENBANK-ID: M86826)(E=2.2e⁻⁴⁰).

A NOV4 polypeptide (SEQ ID NO:8) encoded by SEQ ID NO:7 is 592 amino acid residues and is presented using the one-letter amino acid code in Table 4B. Signal P, Psort and/or Hydropathy results predict that NOV4 has a signal peptide and is likely to be localized to the plasma membrane with a certainty of 0.4600. The most likely cleavage site for a NOV4 peptide is between amino acids 24 and 25, at. AGG-CP. NOV4 has a molecular weight of 64880.1 Daltons.

TABLE 4B
NOV4 protein sequence

| | |
|---|---|
| MTCWLCVLSLPLLLLPAAPPPAGGCPARCECTVQTRAVACTRRRLTAVPDGIPAETRLLELSRNRIR | (SEQ ID NO:8) |
| CLNPGDLAALPALEELDLSENAIAHVEPGAFANLPRLRVLRLRGNQLKLIPPGVFTRLDNLTLLDLS | |
| ENKLVILLDYTFQDLHSLRRLEVGDNDLVFVSRRAFAGLLALEELTLERCNLTALSGESLGHLRSLG | |
| ALRLRHLAIASLEDQNFRRLPGLLHLEIDNWPLLEEVAAGSLRGLNLTSLSVTHTNITAVPAAALRH | |
| QAHLTCLNLSHNPISTVPRGSFRDLVRLRELHLAGALLAVVEPQAFLGLRQIRLLNLSNNLLSTLEE | |
| STFHSVNTLETLRVDGNPLACDCRLLWIVQRRKTLNFDGRLPACATPAEVRGDALRNLPDSVLFEYF | |
| VDRKPKIRERRLQRVTATAGEDVRFLCRAEGEPAPTVAWVTPQHRPVTATSAGRARVLPGGTLEIQD | |
| ARPQDSGTYTCVASNAGGNDTYFATLTVRPEPAANRTPGEAHNETLAALRAPLDLTTILVSTAMGCI | |
| TFLGVVLFCFVLLFVWSRGRGQHKNNFSVEYSFRKVDGPAAAAGQGGARKFNMKMI | |

The disclosed NOV4 amino acid sequence has 327 of 601 amino acid residues (54%) identical to, and 424 of 601 amino acid residues (70%) similar to, the 614 amino acid residue hypothetical 69.2 kDa protein from Macaca fascicularis (BAB03557)(E=3.6e⁻¹⁶⁶).

The disclosed NOV4 protein maps to chromosome 19 and is expressed in at least the following tissues: brain (specifically cerebellum), fetal lung, testis and B-cells. In addition, the sequence is predicted to be expressed in the following tissues because of the expression pattern of SPTREMBL-ID: 073675, a closely related neuronal leucine-rich repeat protein homolog in species Xenopus laevis: brain, eye and spinal cord. Further, the expression pattern of GenBank ID:M86826, another closely related leucine-rich repeat protein, suggests that the protein disclosed in this invention might be predicted to be expressed in the liver. It is also predicted to be expressed in uterus, different regions of the brain, cell lines derived from brain and lung tumors, ovarian tumors, bladder tumors and an ocular metastasis of a melanoma tumor because of the expression pattern of a closely related novel leucine-rich repeat protein gene 20760813_EXT.

NOV4 also has homology to the amino acid sequences shown in the BLASTP data listed in Table 4C.

TABLE 4C
BLAST results for NOV4

| Gene Index/ | | Length | Identity | Positives | |
|---|---|---|---|---|---|
| Identifier | Protein/Organism | (aa) | (%) | (%) | Expect |
| gi\|12309630\|emb\| | 5- | 606 | 339/603 | 439/603 | 0.0 |
| CAC22713.1\|(AL353746) | hydroxytryptamine | | (56%) | (72%) | |
| bA438B23.1 | receptor 7, | | | | |
| (neuronal leucine- | isoform a; | | | | |
| rich repeat | serotonin 5-HT-7 | | | | |
| protein) [Homo | receptor [Homo | | | | |
| sapiens] | sapiens] | | | | |
| gi\|15029689\|gb\| | 5- | 614 | 326/605 | 421/605 | 1e−168 |
| AAH11057.1\|AAH11057 | hydroxytryptamine7 | | (53%) | (60%) | |
| (BC011057) Unknown | receptor isoforn | | | | |
| (protein for | d; serotonin 5-HT- | | | | |
| MGC:17422) [Homo | 7 receptor [Homo | | | | |
| sapiens] | sapiens] | | | | |
| gi\|9651089\|dbj\| | 5- | 614 | 325/605 | 421/605 | 1e−167 |
| BAB03557.1 \|(AB046639) | hydroxytryptainine | | (53%) | (68%) | |
| hypothetical | receptor 7, | | | | |
| protein [Macaca | isoform b; | | | | |
| fascicularis] | serotonin 5-HT-7 | | | | |
| | receptor [Homo | | | | |
| | sapiens] | | | | |
| gi\|12832048\|dbj\| | 5- | 614 | 325/605 | 419/605 | 1e−167 |
| BAB32403.1 \|(AK027262) | HYDROXYTRYPTANINE | | (53%) | (68%) | |
| putative [Mus | 7 RECEPTOR (5-HT- | | | | |
| musculus] | 7) (5-HT-X) | | | | |
| | (SEROTONIN | | | | |
| | RECEPTOR) (SHT7) | | | | |
| | (GPRFO) | | | | |
| gi\|14754729\|ref\|XP_047947.1\| | serotonin receptor | 315 | 159/314 | 211/314 | 4e−75 |
| hypothetical protein FLJ14594 | 7 [Rattus | | (50%) | (66%) | |
| [Homo sapiens] | norvegicus] | | | | |

The homology of these sequences is shown graphically in the ClustalW analysis shown in Table 4D.

[image]

[image]

[image]

Tables 4E-I list the domain description from DOMAIN analysis results against NOV4. This indicates that the NOV4 sequence has properties similar to those of other proteins known to contain this domain.

TABLE 4E
Domain Analysis of NOV4

gnl|Smart|smart00409, IG, Immunoglobulin (SEQ ID NO:47)
Length = 86 residues, 97.7% aligned
Score = 71.6 bits (174), Expect = 1e−13

| | | | |
|---|---|---|---|
| | | 10 20 30 40 50 | |
| NOV4: | 415 | QRVTATAGEDVRFLCRAEGEPAPTVAWVTPQHRPVTATSAGRARVLPG-GTLEIQDARPQ | 473 |
| | | \|\| \|\| \| \| \| \| \| \|\|\| \| + + + \| \|\| \| + \|+ | |
| smart00409: | 2 | PSVTVKEGESVTLSCEASGNPPPTVTWYKQGGKLLAESGRFSVSRSGGNSTLTISNVTPE | 61 |
| NOV4: | 474 | DSGTYTCVASNAGGNDTYFATLTV | 497 |
| | | \|\|\|\|\|\|\| \|+\|+ \|+ + \|\|\|\| | |
| smart00409: | 62 | DSGTYTCAATNSSGSASSGVTLTV | 85 |

TABLE 4F
Domain Analysis of NOV4

gnl|Smart|smart00408, IGc2, Immunoglobulin C-2 Type (SEQ ID NO:48)
Length = 63 residues, 96.8% aligned
Score = 58.2 bits (139), Expect = 1e−09

| | | | |
|---|---|---|---|
| NOV4: | 422 | GEDVRFLCRAEGEPAPTVAWVTPQHRPVTATSAGRARVLPGGTLEIQDARPQDSGTYTCV | 481 |
| | | \|\| \| \| \| \|+\| \| + \|+ \| \| \|\| \|++ +\|\|\| \|\|\|\| | |
| smart00408: | 3 | GESVTLTCPASGDPVPNITWLKDGKP-----LPESRVVASGSTLTIKNVSLEDSGLYTCV | 57 |
| NOV4: | 482 | ASNAGG | 487 |
| | | \| \|+ \| | |
| smart00408: | 58 | ARNSVG | 63 |

TABLE 4G
Domain Analysis of NOV4

gnl|Pfam|pfam00047, ig, Immunoglobulin domain. (SEQ ID NO:49)
Length = 68 residues. 100.0% aligned
Score = 43.9 bits (102), Expect = 2e−05

| | | | |
|---|---|---|---|
| NOV4: | 422 | GEDVRFLCRAEGEPAPTVAWVTPQHRPVTATSAGRARVLPGG-------TLEIQDARPQD | 474 |
| | | \|\| \| \| \| \| + + + +\|\| \|\| +\| \| \|+\| | |
| pfam00047: | 1 | GESVTLTCSVSGYPPDPTVTWLRNGKGIELLGSSESRVTSGGRFSISSLSLTISSVTPED | 60 |
| N0V4: | 475 | SGTYTCVA | 482 |
| | | \|\|\|\|\|\|\| | |
| pfam00047: | 61 | SGTYTCVV | 68 |

TABLE 4H
Domain Analysis of NOV4

gnl|Smart|smart00013, LRRNT, Leucine rich repeat
N-terminal domain (SEQ ID NO:50)
Length = 34 residues, 100.0% aligned
Score = 38.1 bits (87), Expect = 0.001

| | | | |
|---|---|---|---|
| NOV4: | 24 | GCPARCECTVQTRAVACTRRRLTAVPDGIPAETRL | 58 |
| | | \|\|\| \| + \| \|\| \|+ \| \|\| \|\| +\|\|+\| \| | |
| smart- | 1 | QCPAPCNCSPGT-AVDCSGRGLTEVPLDLPADTTL | 34 |
| 00013: | | | |

TABLE 41
Domain Analysis of NOV4

gnl|Smart|smart00082, LRRCT, Leucine rich repeat C-terminal
domain (SEQ ID NO:51)
Length = 51 residues, 92.2% aligned
Score = 358 bits (81), Expect = 0.007

| | | | |
|---|---|---|---|
| NOV4 | 352 | NPLAODCRLLWIVQ-RRKTLNFDGPLPA-CATPAEVRGDALRNLPDS | 396 |
| | | \|\| \|\|\| \| \|+++ + + \|\|+\| +\|\| \|\| \| | |
| smart00082: | 1 | NPFICDCELRWLLRWLEANRHLQDPVDLRCASPESLRGPLLLLLPSS | 47 |

Patp BLAST results for NOV4 include those listed in Table 4J.

TABLE 4J
Patp alignments of NOV4

| | | | Smallest |
|---|---|---|---|
| | | | Sum |
| Sequences producing High-scoring | Reading | High | Prob. |
| Segment Pairs: | Frame | Score | P(N) |
| patp:AAB74705 H. membrane assoc. | +2 | 1621 | 8.9e−166 |
| protein MEMAP-11 [Homo sap], 620 aa | | | |

Leucine rich repeat proteins are a family of proteins characterized by a structural motif rich in leucine residues. They are either transmembrane or secreted proteins and are involved in protein-protein interactions. Members of this family have been implicated in extracellular matrix assembly and cellular growth. In addition, several proteins belonging to this family, such as slit, Toll and robo have been shown to mediate key roles in central nervous system development and organogenesis in Drosophila. Vertebrate orthologs of these proteins have also been shown to have similar roles in the CNS as well as other organ systems like kidney.

The protein with closest homology to the protein of invention is a protein encoded by a cDNA cloned from macaque cerebellum. The next closest member is a protein expressed by a gene that is amplified and overexpressed in malignant glioblastomas. This protein is also coexpressed in a subset of malignant gliomas expressing high levels of MDM-4, a putative proto-oncogene that binds to p53 and may play a role in the mechanism by which these tumors escape growth control. Another related protein is a neural leucine-rich repeat protein that is expressed in the developing eye, brain and spinal cord in Xenopus. It is hypothesized to be involved in neural cell adhesion processes. The protein of the invention has high homology to another novel leucine rich repeat protein (20760813_EXT) discovered at CuraGen that is expressed in the uterus, different regions of the brain, cell lines derived from brain and lung tumors, ovarian tumors, bladder tumors and an ocular metastasis of a melanoma tumor. Leucine-rich repeats are short sequence motifs present in a number of proteins with diverse functions and cellular locations. All proteins containing these repeats are thought to be involved in protein-protein interactions. The crystal structure of ribonuclease inhibitor protein has revealed that leucine-rich repeats correspond to beta-alpha structural units. These units are arranged so that they form a parallel beta-sheet with one surface exposed to solvent, so that the protein acquires an unusual, nonglobular shape. These two features may be responsible for the protein-binding functions of proteins containing leucine-rich repeats. (Kobe and Deisenhofer, Trends Biochem Sci 19(10):415-21, 1994)

Small leucine-rich proteoglycans belong to an expanding gene class whose distinctive feature is a structural motif, called the leucine-rich repeat, found in an increasing number of intracellular and extracellular proteins with diverse biological attributes. Three-dimensional modeling of their prototype protein core proposes a flexible, arch-shaped binding surface suitable for strong and distinctive interactions with ligand proteins. Changes in the properties of individual proteoglycans derive from amino acid substitutions in the less conserved surface residues, changes in the number and length of the leucine-rich repeats, and/or variation in glycosylafion These proteoglycans are tissue organizers, orienting and ordering collagen fibrils during ontogeny and in pathological processes such as wound healing, tissue repair, and tumor stroma formation. These properties are rooted in their bifunctional character: the protein moiety binding collagen fibrils at strategic loci, the microscopic gaps between staggered fibrils, and the highly charged glycosaminoglycans extending out to regulate interfibrillar distances and thereby establishing the exact topology of fibrillar collagens in tissues. These proteoglycans also interact with soluble growth factors, modulate their functional activity, and bind to cell surface receptors. The latter interaction affects cell cycle progression in a variety of cellular systems and could explain the purported changes in the expression of these gene products around the invasive neoplastic cells and in regenerating tissues. (Tozzo, Crit Rev Biochem Mol Biol 32(2):141-74, 1997) Many members of the leucine-rich repeat superfamily have been investigated.

Two-dimensional electrophoresis of enzyme-digested genomic DNA was used to identify a novel gene GAC1, which maps at 1q32.1 and which is overexpressed in malignant gliomas in which it is amplified. GAC1 encodes a protein which belongs to the leucine-rich repeat superfamily. Amplification and overexpression of GAC1 was demonstrated in two of eight tumors where amplifications were previously evidenced by comparative genomic hybridization (one glioblastoma multiforme and one anaplastic astrocytoma), and in one of eight unselected glioblastomas multiforme. GAC1 exhibits sequence homology with other proteins which function as cell-adhesion molecules or as signal transduction receptor and is a likely candidate for the target gene in the 1q32.1 amplicon in malignant gliomas. (Almeida et al., Oncogene 16(23):2997-3002, 1998)

Reifenberger et al. have previously reported on the amplification and overexpression of the MDM2 proto-oncogene in a subset of malignant gliomas without TP53 mutation (G. Reifenberger et al., Cancer Res 53: 2736-2739, 1993). In a more recent study it was shown that the MDM4 (MDMX) gene located on 1q32 is a further target for amplification in malignant gliomas. MDM4 codes for a Mdm2-related protein that can bind to p53 and inhibits p53-mediated transcriptional transactivation. A series of 208 gliomas (106 glioblastomas, 46 anaplastic gliomas, and 56 low-grade gliomas) was investigated and 5 tumors (4 glioblastomas and 1 anaplastic oligodendroglioma) with MDM4 amplification and overexpression were identified. Several other genes from 1q32 were found to be coamplified with MDM4, such as GAC 1 in five tumors, REN in four tumors, and RBBP5 in three tumors. Additional analyses revealed that the malignant gliomas with MDM4 amplification and overexpression carried neither mutations in conserved regions of the TP53 gene nor amplification of the MDM2 gene. Taken together, these data indicate that amplification and overexpression of MDM4 is a novel molecular mechanism by which a small fraction of human malignant gliomas escapes p53-dependent growth control. (Riemenschneider et al., Cancer Res 59(24):6091-6, 1999) Hayata et al. reported the isolation and characterization of a Xenopus sequence, XNLRR-1, that is closely related to a gene for mouse neuronal leucine-rich repeat protein (NLRR-1).(Hayata et al., Gene 221(1):159-66, 1998) The cDNA clone is 4179 bp long and encodes a putative transmembrane glycoprotein of 718 amino acids, containing 12 leucine-rich repeats followed by one C2-type immunoglobulin-like domain and one fibronectin type-III repeat. XNLRR-1 is transcribed mainly in the developing eye area and the ventricular zone from diencephalon to hindbrain and slightly in spinal cord in Xenopus tadpoles. The similarity of the XNLRR-1 gene to other known cell adhesion molecules, together with the expression pattern, suggests that XNLRR-I is involved in interactions at the neuronal cell surface. (Hayata et al., Gene 221(1):159-66, 1998)

The slit (sli) gene, encoding a secreted glycoprotein, has been demonstrated to play a vital role in axonal guidance in Drosophila melanogaster by acting as a signalling ligand for the robo receptor (Rothberg et al., Genes Dev. 4, 2169-2187, 1990; Kidd et al., Cell 96, 785-794, 1999). Multiple homologs of both sli and robo have been identified in vertebrates and are thought to play similar roles to their fly counterparts in neural development (Brose et al., Cell 96, 795-806, 1999). Slit2 has been shown to bind Robo1, mediating both neuronal and axonal guidance in the developing central nervous system (CNS), (Brose et al., 1999; Hu, Neuron 23, 703-711, 1999). Importantly, both gene families display distinct expression patterns outside the CNS (Holmes et al., Mech. Dev. 79, 57-72, 1998; Yuan et al., Dev. Biol. 212, 290-306, 1999). Using in situ hybridization on metanephric explant cultures and urogenital tract sections, the expression patterns of Slit 1, 2, 3 and Robo1 and 2 were investigated during murine metanephric development. Slit1 was expressed in the metanephric mesenchyme (MM) surrounding the invading ureteric tree (UT). Slit2 was expressed at the tips of the UT and both Slit2 and Slit3 were expressed at the far proximal end of the comma shaped and S-shaped bodies. Expression of Robo1 was initially diffuse throughout the MM, then upregulated in the pretubular aggregates, and maintained at the distal end of the comma and S-shaped bodies. Robo2 was detected in the induced MM surrounding the arborizing UT tips and later in the proximal end of the S-shaped bodies. Coincident expression of Robo1 with Slit1 in the metanephric mesenchyme and Robo2, Slit2 and Slit3 in the far proximal end of the S-shaped bodies was observed during metanephric development. (Piper et al., Mech Dev 94(1-2):213-217, 2000)

Loss of heterozygosity for 10q23-26 is seen in over 80% of glioblastoma multiforme tumors. Chemova et al. have used a positional cloning strategy to isolate a novel gene, LGI1 (Leucine-rich gene-Glioma Inactivated), which is rearranged as a result of the t(10;19)(q24;q13) balanced translocation in the T98G glioblastoma cell line lacking any normal chromosome 10. Rearrangement of the LGI1 gene was also detected in the A172 glioblastoma cell line and several glioblastoma tumors. These rearrangements lead to a complete absence of LGI1 expression in glioblastoma cells. The LGI1 gene encodes a protein with a calculated molecular mass of 60 kD and contains 3.5 leucine-rich repeats (LRR) with conserved flanking sequences. In the LRR domain, LGI1 has the highest homology with a number of transmembrane and extracellular proteins which function as receptors and adhesion proteins. LGI1 is predominantly expressed in neural tissues, especially in brain; its expression is reduced in low grade brain tumors and it is significantly reduced or absent in malignant gliomas. Its localization to the 10q24 region, and rearrangements or inactivation in malignant brain tumors, suggest that LGI1 is a candidate tumor suppressor gene involved in progression of glial tumors. (Chemova et al., Oncogene 17(22):2873-81, 1998)

Nearly all of the insulin-like growth factor (IGF) in the circulation is bound in a heterotrimeric complex composed of IGF, IGF-binding protein-3, and the acid-labile subunit (ALS). Full-length clones encoding ALS have been isolated from human liver cDNA libraries by using probes based on amino acid sequence data from the purified protein. These clones encode a mature protein of 578 amino acids preceded by a 27-amino acid hydrophobic sequence indicative of a secretion signal. Expression of the cDNA clones in mammalian tissue culture cells results in the secretion into the culture medium of ALS activity that can form the expected complex with IGF-I and IGF-binding protein-3. The amino acid sequence of ALS is largely composed of 18-20 leucine-rich repeats of 24 amino acids. These repeats are found in a number of diverse proteins that, like ALS, participate in protein-protein interactions. (Leong et al., Mol Endocrinol 6(6):870-6, 1992)

After birth, the acid-labile subunit (ALS) associates in the circulation with insulin-like growth factor (IGF)-I or -II and with IGF binding protein-3 (IGFBP-3) to form a 150-kilodalton complex. This association leads to the retention of IGFs in the vascular system and promotes their endocrine actions. ALS is synthesized almost exclusively in liver, and both hepatic ALS mRNA and circulating levels are increased by growth hormone (GH). Three major areas of study were pursued to better understand the regulation of ALS synthesis and its role in the circulating IGF system. First, the mouse ALS gene was isolated and shown to be organized into two exons and a single intron on chromosome 17. Second, using transient transfection studies in the rat H4-II-E hepatoma cell line and primary rat hepatocytes, thc region of the mouse promoter that is responsive to GH was mapped to a nine-base pair cis-element resembling a gamma-interferon-activated sequence. The activation of the mouse ALS gene by GH is mediated by the binding of STAT5 isoforms to this sequence. Finally, an ALS knockout model was created by inactivating the ALS gene in mouse embryonic stem cells. Mice that are homozygous for the mutation grow at a slower rate after birth. This growth depression is associated with large decreases in the plasma concentrations of both IGF-I and IGFBP-3, indicating the critical role of ALS in the regulation of circulating levels of these proteins. Studies of this model will lead to a better understanding of the circulating IGF system. (Boisclair et al., Pediatr Nephrol 14(7):562-6, 2000)

The protein similarity information, expression pattern, and map location for the leucine-rich repeat proteins-like protein and nucleic acid (NOV4) disclosed herein suggest that this NOV4 protein may have important structural and/or physiological functions characteristic of the leucine-rich repeat family. Therefore, the NOV4 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV4 nucleic acids and proteins of the invention are useful in potential diagnostic and therapeutic applications implicated in various diseases and disorders described below and/or other pathologies. For example, the compositions of the present invention will have efficacy for treatment of patients suffering from Von Hippel-Lindau (VHL) syndrome, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies behavioral disorders, addiction, anxiety, pain, neuroprotection, systemic lupus erythematosus, autoimmune disease, asthma, emphysema, scleroderma, allergy, ARDS, fertility, ocular disorders, glioblastoma, glioma, uterine tumors, melanoma, bladder tumors, lung tumors etc. The NOV4 nucleic acids, or fragments thereof, may further be useful in diagnostic applications, wherein the presence or amount of the nucleic acid or the protein are to be assessed.

NOV4 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example, the disclosed NOV4 protein has multiple hydrophilic regions, each of which can be used as an immunogen. These novel proteins can be used in assay systems for functional analysis of various human disorders, which will help in understanding of pathology of the disease and development of new drug targets for various disorders.

NOV5

A disclosed NOV5 nucleic acid of 771 nucleotides (also referred to as 51624520A1/dj1160k1_A1) encoding a novel CD-81-like protein is shown in Table 5A. An open reading frame was identified beginning with an ATG initiation codon at nucleotides 5-7 and ending with a TAG codon at nucleotides 749-751. A putative untranslated region upstream from the initiation codon and downstream from the termination codon is underlined in Table 5A, and the start and stop codons are in bold letters.

TABLE 5A
NOV5 Nucleotide Sequence

| | |
|---|---|
| TACCATGGAGGCGACTGTCTGAGCTGCATGAAGTATCTGATGTTTGTATTCAATT | (SEQ ID NO:9) |
| TCTTCATATTTCTGGGCGGGGCCTGCCTGCTGGCCATCGGCATCTGGGTCATGGTG | |
| GACCCCACCGGCTTCCGGGAQATCGTGGCTGCCAATCCTCTGCTCCTCACCGGCCC | |
| CTACATCCTCCTGGCCATGGGGGGCCTGCTCTTTCTGCTCGGCTTCCTGGGCTGCT | |
| GCGGGGCCGTCCGTGAGAACAAGTGTCTGCTGCTATTTTTCTTCCTGTTCATCCTG | |
| ATCATCTTCCTGGCAGAGCTCTCAGCAGCCATCCTGGCCTTCATCTTCAGGGAAAA | |
| TCTCACCCGAGAATTCTTCACCAAGGAGCTCACCAAGCACTACCAGGGCAATAACG | |
| ACACAGACGTCTTCTCTGCCACCTGGAACTCGGTCATGATCACATTTGGTTGCTGC | |
| GGGGTCAACGGGCCTGAAGACTTTAAGTTTGCATCTGTGTTTCGACTCCTGACCCT | |
| GGATAGTGAAGAGGTGCCGGAGGCCTGCTGCCGGAGGGAACCCCAAAGTCGGGACG | |
| GGGTCCTGCTGAGCCGGGAGGAGTGCCTCCTGGGAAGGAGCCTATTCCTAAACAAG | |
| CAGGGCTGTTACACGGTGATCCTCAACACCTTCGAGACCTACGTCTACTTGGCCGG | |
| AGCCCTTGCCATCGGGGTACTGGCCATCGAGCTTTTCGCCATGATCTTTGCCATGT | |
| GCCTCTTCCGGGGCATCCAGTAGAGGGTATGGCCTGAAGCCTA | |

The NOV5 sequence was identified by subjecting Acc. No. GM_{—}51624520_A to an exon linking process. PCR primers were designed to Ace. No. GM_{—}51624520_A by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a library containing a wide range of cDNA species. The resulting amplicon was gel purified, cloned and sequenced to high redundancy to provide the NOV5 sequence (GM_{—}51624520A1/dj1160k1_A1).

The NOV5 nucleic acid was identified on chromosome 11 by TblastN using CuraGen Corporation's sequence file for CD-81 or homolog as run against the Genomic Daily Files made available by GenBank or from files downloaded from the individual sequencing centers. The nucleic acid sequence was predicted from the genomic file Genbank Accession Number: AC016702 by homology to a known CD-81 or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

A disclosed NOV5 polypeptide (SEQ ID NO:10) encoded by SEQ ID NO:9 is 247 amino acid residues and is presented using the one-letter code in Table 5B. Signal P, Psort and/or Hydropathy results predict that NOV5 has a signal peptide and is likely to be localized in the plasma membrane with a certainty of 0.6400. The most likely cleavage site for a NOV5 peptide is between amino acids 27 and 28, at: ACL-LA. NOV5b has a molecular weight of 27709.5 Daltons.

TABLE 5B
Encoded NOV5 protein sequence

| | |
|---|---|
| MEGDCLSCMKYLMFVFNFFIFLGGACLLAIGIWVMVDPTGFREIVAANPLLLTGAYILLAMGGLL | (SEQ ID NO:10) |
| FLLGFLGCCGAVRENKCLLLFFFLFILIIFLAELSAAILAFIFRENLTREFFTKELTKHYQGNND | |
| TDVFSATWNSVMITFGCCGVNGPEDFKFASVFRLLTLDSEEVPEACCRREPQSRDGVLLSREECL | |
| LGRSLFLNKQGCYTVILNTFETYVYLAGALAIGVLAIELFAMIFAMCLFRGIQ | |

The disclosed NOV5 amino acid sequence has 201 of 248 amino acid residues (81%) identical to, and 220 of 248 residues (88%) positive with, the 247 amino acid residue neuronal tetraspanin protein from Gallus gallus (chicken) (ptnr:SPTREMBL-ACC:Q9PTE0)(E=1.1e⁻¹⁰⁷)

NOV5 also has homology to the amino acid sequences shown in the BLASTP data listed in Table 5C.

TABLE 5C
BLAST results for NOV5

| Gene Index/ | | Length | Identity | Positives | |
|---|---|---|---|---|---|
| Identifier | Protein/Organism | (aa) | (%) | (%) | Expect |
| gi\|14042602\|dbj\| | unnamed protein | 248 | 215/236 | 219/236 | 4e−47 |
| BAB55318.1\| | product [Homo | | (91%) | (92%) | |
| (AK027715) | sapiens] | | | | |
| gi\|6601561\|gb\| | neuronal | 247 | 180/235 | 199/235 | 2e−80 |
| AAF19031.1\|AF206661 | tetraspanin | | (76%) | (84%) | |
| 1 (AF206661) | [Gallus gallus] | | | | |
| gi\|14767870\|ref\| | similar to | 214 | 184/204 | 186/204 | 3e−69 |
| XP_029348.1\| | uroplakin 1B; | | (90%) | (90%) | |
| | tetraspan (H. | | | | |
| | sapiens) [Homo | | | | |
| | sapiens] | | | | |

The homology of these sequences is shown graphically in the ClustalW analysis shown in Table 5D.

[image]

Table 5E lists the domain description from DOMAIN analysis results against NOV5. This indicates that the NOV5 sequence has properties similar to those of other proteins known to contain this domain.

TABLE 5E
Domain Analysis of NOV5

gnl|Pfam|pfam00335, transmembrane4, Tetraspanin family. (SEQ ID NO:55)
Length = 223 residues, 82.5% aligned
Score = 82.4 bits (202), Expect = 3e−17

| | | | |
|---|---|---|---|
| NOV5: | 10 | KYLMFVFNFFIFLGGACLLAIGIWVMVDPTGFREIVAANPLLLTGAYILLAMGGLLFLLG | 69 |
| | | \|\|\|+\|+ \|\| +\| \| \|\|\|+\|\|\|++\|\| + \| \|++ + \| \|\|+\|+\|+\| +\|\|\|+\| | |
| Pfam00335: | 1 | KYLLFLLNFLFWLCGILLLAVGIWLLVDKSFFSELLGGSLSNLVAAYVLIAVGIILFLVG | 60 |
| NOV5: | 70 | FLGCCGAVRENKCLLLFFFLFILIIFLAELSAAILAFIFRENLTREFFTKGLTKHYQGNN | 129 |
| | | \|\|\|\|\|\|\|+\|\|++\|\|\| +\|+\|+\|\|+\|+ \|\|+\| \|\|\|\|+\|\|+ \| | |
| Pfam00335: | 61 | FLGCCGAIRESRCLLGLYFVFLLILFILELAAGILAFVFRDQLESSLKESLKKAIKNYYG | 120 |
| NOV5: | 130 | DTDVFSATWNSVMITFGCCGVNGPEDFKFAPWIVKR-----CRRLLPEEPQSRDGVLLSR | 184 |
| | | \|+ + \| \|\|\|\|\|\| \|+ + \| \|+ \| + + | |
| Pfam00335: | 121 | TDPDERNAWDKLQEQFKCCGVNGYTDWFDSQWFSNGVPFSCCKPSLSCNSAQDEEDTIYL | 180 |
| NOV5: | 185 | EECL | 188 |
| | | \|\| | |
| Pfam00335: | 181 | RGCL | 184 |

Patp BLAST results for NOV5 include those listed in Table 5F.

TABLE 5F
Patp alignments of NOV5

| | | | Smallest |
|---|---|---|---|
| | | | Sum |
| Sequences producing High-scoring | Reading | High | Prob. |
| Segment Pairs: | Frame | Score | P (N) |
| patp:AAB93282 Protein sequence SEQ ID | +2 | 1295 | 3.1e−131 |
| NO:12330 [Homo sap], 248 aa | | | |
| patp:AAB88457 Membrane/secretory | +2 | 1226 | 6.4e−124 |
| clone PSEC0247 [Homo sap], 236 aa | | | |
| patp:AAB49503 Clone HCE1K90 | | | |
| #1 [Homo sap], 248 aa | | | |

Possible SNPs found for NOV5 are listed in Table 5G.

TABLE 5G
SNPs

| Consensus | Base | Amino Acid | Amino Acid |
|---|---|---|---|
| Position | Change | Position | Change |
| 295 | A > G | Silent | N/A |
| 372 | A > G | 123 | K > R |
| 401 | G > A | 133 | V > I |

CD81 antigen (or TAPA1) is a 26-kD integral membrane protein expressed on many human cell types. Antibodies against TAPA1 induce homotypic aggregation of cells and can inhibit their growth. Oren et al. isolated a cDNA coding for TAPA1.(Oren et al., Mol Cell Biol 10(8):4007-15, 1990) The highly hydrophobic TAPA1 protein contains four putative transmembrane domains and a potential N-myristoylation site. TAPA1 showed strong homology with the CD37 leukocyte antigen (OMIM-151523) and with the ME491 melanoma-associated antigen (OMIM-155740), both of which have been implicated in the regulation of cell growth. Andria et al. cloned the murine homolog of TAPA1 from both cDNA and genomic DNA libraries and demonstrated a very high level of homology between human and mouse genes. (Andria et al., J Immunol 147(3):1030-6, 1991; OMIM: 186845)

CD81 is a member of the transmembrane pore integral membrane protein family. It has broad tissue distribution, but its function had not been identified. Boismenu et al. obtained a complete gene from mouse CD81 by RT-PCR. (Boismenu et al., Science. 271(5246):198-200, 1996) A monoclonal antibody specific for mouse CD81 blocked the appearance of alpha-beta T cells but not gamma-delta T cells in fetal organ cultures initiated with day 14.5 thymus lobes. In re-aggregation cultures with CD81-transfected fibroblasts, CD4−/CD8-thymocytes differentiated into CD4+/CD8+T cells. The authors therefore concluded that interaction between immature thymocytes and stromal cells expressing CD81 are required and may be sufficient to induce early events associated with T-cell development.

Chronic hepatitis C virus (HCV) infection occurs in about 3% of the world's population and is a major cause of liver disease. HCV infection is also associated with cryo-globulinemia, a B lymphocyte proliferative disorder. Virus tropism and the mechanisms of cell entry are not completely understood. Pileri et al. demonstrated that the HCV envelope protein E2 binds human CD81, a tetraspanin expressed on various cell types including hepatocytes and B lymphocytes. (Pileri et al., Science;282(5390):93841, 1998) Binding of E2 was mapped to the major extracellular loop of CD81. Recombinant molecules containing this loop bound HCV and antibodies that neutralize HCV infection in vivo inhibited virus binding to CD81 in vitro.

Through eukaryotic expression cloning with an antimetastatic monoclonal antibody Testa et al. have recently identified a tetraspanin member, PETA-3/CD151, as an effector of human tumor cell migration and metastasis. (Testa et al., Cancer Res 59(15):3812-20, 1999)

The above defined information for this invention suggests that this CD-81-like protein (NOV5) may function as a member of a “CD-81 family”. Therefore, the NOV5 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV5 nucleic acids and proteins of the invention are useful in potential therapeutic applications implicated in HCV infection, Burkitt Lymphoma, and metastatic tumors, immunological disorders particularly those involving T-cells, and/or other pathologies and disorders. For example, a cDNA encoding the CD-81-like protein may be useful in gene therapy, and the CD-81-like protein may be useful when administered to a subject in need thereof. By way of nonlimiting example, the compositions of the present invention will have efficacy for treatment of patients suffering from HCV infection, Burkitt Lymphoma metastatic tumors and immunological disorders particularly those involving T-cells. The NOV5 nucleic acid, or fragments thereof, may further be useful in diagnostic applications, wherein the presence or amount of the nucleic acid or the protein are to be assessed.

NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein has multiple hydrophilic regions, each of which can be used as an immunogen. In one embodiment, a contemplated NOV5 epitope is from about amino acids 110 to 140. In another embodiment, a NOV5 epitope is from about amino acids 170 to 190. This novel protein also has value in development of powerful assay system for functional analysis of various human disorders, which will help in understanding of pathology of the disease and development of new drug targets for various disorders.

NOV6

NOV6 includes two novel Voltage-Dependent Anion Channel-like proteins disclosed below. The disclosed proteins have been named NOV6a and NOV6b.

NOV6a

A disclosed NOV6a nucleic acid of 923 nucleotides (also referred to as GM_AC011898_A) encoding a novel Voltage-Dependent Anion Channel-like protein is shown in Table 6A. An open reading frame was identified beginning with an ATG initiation codon at nucleotides 9-11 and ending with a TAA codon at nucleotides 882-884. A putative untranslated region upstream from the initiation codon and downstream from the termination codon is underlined in Table 6A, and the start and stop codons are in bold letters.

TABLE 6A
NOV6a Nucleotide Sequence

| | |
|---|---|
| AGCATAAGATGGCTGTGCCACCCATGTATGCCAATCTTGGCAAGTCTGCCAGGGATGTCTTCA | (SEQ ID NO:11) |
| CTAAGGGCTATGGATTTGGCTTAATAATGCTTGATTTGAAAACAAAATCTGAGAATGGGTTGG | |
| AATTTACAAGCTCAGGCTCAGCCAACACTGAGACCACCAAAGTGACGGGCAGTCTGGAAACCA | |
| AGTACAGATGGACTGAGTACGGCCTGACGTTTACAGAGAAATACAACACGGATAATACACTAG | |
| GCACCGAGATTACTGTGGAAGATCAGCTTGCACGTGGACTGAAGCTGACCTTCGATTCATCCT | |
| TCTTACCTAAGACTGGGGGAAAAAAGATGCTAAAAGGGAAAAAAGATGCTAAAATCAAGACAG | |
| GTTACAAGCAGGAGCACATTACCCTGGACTGCAACATAGATTTCGACATTGCTGGGCCTTCCA | |
| TCCGGGCTGCTCTGATGCTGGGTTACAAGGGCTGGCTGGCCGGCTACCAGATGAATTTTGAGA | |
| CTGCAAAGTCCGGAGTGACCCAGAGCAACTTTGCAGTTGGCTGCAAGACTGATGAATTCCAAT | |
| AGTTGGAGACTGCTGTCAATCTCACCTGGACAAGCGCAGGAAACAGTAACACGCATTTCAAAA | |
| TAGCAGCCAAGTATTTGATTGACCCTGAAGCCTGCTTCTTGGCTAAAGTGAACAACTCCAGCC | |
| TGATAGGTTTAGGATACACTCAGACCCTAAAGCCAGGTATCAAACTGACACTGTCAGCTCTTC | |
| TGGATGGCAAGAACGTCAATGCTGGTGGCCACAAGCTTGGTCTAGGACTGGAATTTCAAGCAT | |
| AAATGAATACTGTACAATTGTTTAATTTTAAACTATTTTGC | |

The NOV6a nucleic acid was identified on chromosome Y by TblastN using CuraGen Corporation's sequence file for Voltage-Dependent Anion Channel or homolog as run against the Genomic Daily Files made available by GenBank or from files downloaded from the individual sequencing centers. The nucleic acid sequence was predicted from the genomic file Genbank or Sequencing Center accession number: AC011898 A by homology to a known Voltage-Dependent Anion Channel or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The disclosed NOV6a nucleic acid sequence has 562 of 573 bases (98%) identical to a Homo sapiens Voltage-Dependent Anion Channel mRNA (GENBANK-ID: AJ002428)(E=1.0e⁻¹⁹¹).

A disclosed NOV6a polypeptide (SEQ ID NO:12) encoded by SEQ ID NO:11 is 291 amino acid residues and is presented using the one-letter amino acid code in Table 6B. Signal P, Psort and/or Hydropathy results predict that NOV6a does not contain a signal peptide and is likely to be localized in the microbody (peroxisome) with a certainty of 0.5118. NOV6a has a molecular weight of 31703.9 Daltons.

TABLE 6B
Encoded NOV6a protein sequence.

(SEQ ID NO:12)

MAVPPMYANLGKSARDVFTKGYGFGLIMLDLKTKSENGLEFTSSGSANTETTKVTGSLETKYRWTEYG
LTFTEKYNTDNTLGTEITVEDQLARGLKLTFDSSFLPKTGGKKMLKGKKDAKIKTGYKQEHITLDCNI
DFDIAGPSIRAALMLGYKGWLAGYQMNFETAKSGVTQSNFAVGCKTDEFQFHTNVNDGTEFGGLIYQK
VNKKLETAVNLTWTSAGNSNTHFKIAAKYLIDPEACFLAKVNNSSLIGLGYTQTLKPGIKLTLSALLD
GKNVNAGGHKLGLGLEFQA

The disclosed NOV6a amino acid sequence has 258 of 291 amino acid residues (88%) identical to, and 267 of 291 residues (91%) positive with, the 283 amino acid residue VOLTAGE-DEPENDENT ANION CHANNEL 1 protein from Oryetolagus cuniculus (Rabbit) (ptnr:SPTREMBL-ACC: Q9TT15)(E=1.2e⁻¹³²). The global sequence homology (as defined by FASTA alignment with the full length sequence of this protein) is 92% amino acid homology and 91% amino acid identity.

NOV6b

A disclosed NOV6b nucleic acid of 867 nucleotides (also referred to as GM_AL133368_A) encoding a novel Voltage-Dependent Anion Channel-like protein is shown in Table 6C. An open reading frame was identified beginning with an ATG initiation codon at nucleotides 5-7 and ending with a TAA codon at nucleotides 854-57. A putative untranslated region upstream from the initiation codon and downstream from the termination codon is underlined in Table 6C, and the start and stop codons are in bold letters.

TABLE 6C
NOV6b Nucleotide Sequence

| | |
|---|---|
| AGATATGTGTAACACACCAACTTACTGTGACCTGGGAAAGGCTGCTGAGGATGTCTTCAACAA | (SEQ ID NO:13) |
| AGGATATGGCTTTGGCATGGGGAAGATAGACCTGAAAACCAAGTCCTGTAGTGCAGTCGAATT | |
| TTCTACTTCTGGTCATGCTTACACTGATACAGGGAAAGCATCAGGCAACCTAGAACCCGAATG | |
| TAAGGTCTGTAACTATGGACTTACCTTCACTCAAAAACGGAACACAGACAATACTCTTGGGAC | |
| AGAAATCTCTTTGGAGAATAAGTTGGCTAAAGGGTTGAAACTGAGTCTTGATACCATATTGGT | |
| ACCAAACACAGGAAAGAAGAGTGGGGAATTGAAGGCCTCCTATAAATGGGATTGTTTTAGTGT | |
| TGGCAGTAATGTTGATCTAGATTTTTCCGGACCAACCATCTATGGCTGGGCTGTGTTGGTCTT | |
| TGAAGGGTGGCTTGCCGGCTATCAGATGAGTTTTGACACAGCCAAATCCAAATTGTCACAGAA | |
| TAATTTCGCCCTGGGTTACGAGGCTGCAGACTTCCAGCTGCACACACATGTGACCGATGGCAC | |
| TGAATTTGGAGGTTCTATCTACCAGAAGGTTAATGGGATTGAAATGTCAATAAACCTTGCTTG | |
| GACGGCTGGAAGTAACAACACCCATTTTGGCATTGCTACTAAGTACAAGCTGGATTGTAGAAC | |
| TTCTCTCTCTGCTAAAGTAAATAATGCCAGCCTGATTGGACTGGGTTATACTCAGACCCTTCG | |
| ACCCGGAGTCAAATTGACCCTTTTATCAGCTTTAATCGATGGAAACAACTTCAGTGCAGGAGG | |
| TCACAAGGTTGGCTTGGCGTTTGAACTGCAAGCTTAATGTGGTTTGAG | |

The NOV6b novel nucleic acid was identified on chromosome 14 by TblastN using CuraGen Corporation's sequence file for Voltage-Dependent Anion Channel or homolog as run against the Genomic Daily Files made available by GenBank or from files downloaded from the individual sequencing centers. The nucleic acid sequence was predicted from the genomic file Genbank or Sequencing Center accession number: AL133368 by homology to a known Voltage-Dependent Anion Channel or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The disclosed NOV6b nucleic acid sequence has 819 of 867 bases (94%) identical to a Homo sapiens Voltage-Dependent Anion Channel mRNA (GENBANK-ID: AF038962)(2.3e⁻¹⁶⁸).

A disclosed NOV6b polypeptide (SEQ ID NO:14) encoded by SEQ ID NO:13 is 283 amino acid residues and is presented using the one-letter amino acid code in Table 6D. Signal P, Psort and/or Hydropathy results predict that NOV6b does not contain a signal peptide and is likely to be localized in the microbody (peroxisome) with a certainty of 0.6113. NOV6b has a molecular weight of 30413.0 Daltons.

TABLE 6D
Encoded NOV6b protein sequence.

(SEQ ID NO:14)

MCNTPTYCDLGKAAEDVFNKGYGFGMGKIDLKTKSCSAVEFSTSGHAYTDTGKASGNLEPECKVCNYG
LTFTQKRNTDNTLGTEISLENKLAKGLKLSLDTILVPNTGKKSGELKASYKWDCFSVGSNVDLDFSGP
TIYGWAVLVFEGWLAGYQMSFDTAKSKLSQNNFALGYEAADFQLHTHVTDGTEFGGSIYQKVNGIEMS
INLAWTAGSNNTHFGIATKYKLDCRTSLSAKVNNASLIGLGYTQTLRPGVKLTLLSALIDGNNFSAGG
HKVGLAFELQA

The disclosed NOV6b amino acid sequence has 256 of 283 amino acid residues (90%) identical to, and 267 of 283 residues (94%) positive with, the 283 amino acid residue VOLTAGE-DEPENDENT ANION CHANNEL 3 protein from Oryctolagus cuniculus (Rabbit) (ptnr:SPTREMBL-ACC: Q9TT13)(4.4e−135). The global sequence homology (as defined by FASTA alignment with the full length sequence of this protein) is 91% amino acid homology and 90% amino acid identity.

NOV6a and NOV6b are related to each other as shown in the alignment listed in Table 6E.

[image]

NOV6a also has homology to the amino acid sequences shown in the BLASTP data listed in Table 6F.

TABLE 6F
BLAST results for NOV6a

| Gene Index/ | | Length | Identity | Positives | |
|---|---|---|---|---|---|
| Identifier | Protein/Organism | (aa) | (%) | (%) | Expect |
| gi\|10720404\|sp\| | VOLTAGE- | 296 | 255/291 | 266/291 | 1e−133 |
| Q60932\|POR1_MOUSE | DEPENDENT | | (87%) | (90%) | |
| | ANION-SELECTIVE | | | | |
| | CHANNEL PROTEIN | | | | |
| | 1 (VDAC-1) | | | | |
| | (MVDAC1) | | | | |
| | (MVDAC5) (OUTER | | | | |
| | MITOCHONDRIAL | | | | |
| | MEMBRANE | | | | |
| | PROTEIN PORIN | | | | |
| | 1) | | | | |
| | (PLASMALEMMAL | | | | |
| | PORIN) | | | | |
| gi\|10720225\|sp\| | VOLTAGE- | 283 | 258/291 | 267/291 | 1e−133 |
| Q9TT15\|POR1_RABIT | DEPENDENT | | (88%) | (91%) | |
| | ANION-SELECTIVE | | | | |
| | CHANNEL PROTEIN | | | | |
| | 1 (VDAC-1) | | | | |
| | (OUTER | | | | |
| | MITOCHONDRIAL | | | | |
| | MEMBRANE | | | | |
| | PROTEIN PORIN | | | | |
| | 1) | | | | |
| gi\|8745552\|gb\|AAF | voltage- | 283 | 257/291 | 267/291 | 1e−133 |
| 78963.1\|AF2684611 | dependent anion | | (88%) | (91%) | |
| (AF268461) | channel 1 [Sus | | | | |
| | scrofa] | | | | |
| gi\|4507879\|ref\|NP | voltage- | 283 | 258/291 | 267/291 | 1e−133 |
| 003365.1\| | dependent anion | | (88%) | (91%) | |
| | channel 1 [Homo | | | | |
| | sapiens] | | | | |
| gi\|238427\|gb\| | Porin 31HM | 282 | 257/290 | 266/290 | 1e−132 |
| AAB20246.1\| | [human, | | (88%) | (91%) | |
| | skeletal muscle | | | | |
| | membranes, | | | | |
| | Peptide, 282 | | | | |
| | aa] | | | | |

The homology of these sequences is shown graphically in the ClustalW analysis shown in Table 6G.

[image]

[image]

Table 6H lists the domain description from DOMAIN analysis results against NOV6a. This indicates that the NOV6a sequence has properties similar to those of other proteins known to contain this domain.

TABLE 6H
Domain Analysis of NOV6a

gnl|Pfam|pfam01459, Euk_porin, Eukaryotic porin. (SEQ ID NO:61)
Length = 281 residues, 99.3% aligned
Score = 303 bits (775), Expect = 1e−83

| | | | |
|---|---|---|---|
| NOV6a | 4 | PPMYANLGKSARDVFTKGYGFGLIMLDLKTKSENGLEFTSSGSANTETTKVTGSLETKYR | 63 |
| | | \|\|+\|+++\|\|\|\|\|\|+ \| \| \|\| + \|\| \|\|+ \|\|+\|\|\|\|+\| \| ++ \|++\|+\|\|\|\|\|+ | |
| Pfam01459: | 3 | PPLYSDIGKSARDLLNKDYNFGPLKFDLTTKTPNGVEFTSAGKQNVDSGKLSGNLETKYK | 62 |
| NOV6a: | 64 | WTEYGLTFTEKYNTDNTLGTEITVEDQLARGLKLTFDSSFLPKTGGKKMLKGKKDAKIKT | 123 |
| | | \|\|\|\| \|+\|+\|\|\|\| \|\|\| \| \|+\|\|\|\| \|\|\|\| \| \|\| \|+\| \|\| | |
| Pfam01459: | 63 | DKNYGLTLTQKWNTDNNLGTTIEVDDQLAPGLKLILDFSFPPQTAKSGKLK--------L | 114 |
| NOV6a: | 124 | GYKQEHITLDCNIDFDIAGPSIRAALMLGYKGWLAGYQMNFETAKSGVTQSNFAVGCKTD | 183 |
| | | \| ++ ++\| + \|\|+\| + +\|\|++\|\|\|\|\| ++\|+\|\| \| \|+ \| \|+\| | |
| Pfam01459: | 115 | QYLHDYFGARASVDL-LKGPTINGSGVLGHEGWLAGADVSFDTATSKFTKYNAALGYTAP | 173 |
| NOV6a: | 184 | EFQFHTNVNDGTEFGGLIYQKVNKKLETAVNLTWTSAGNSNTHFKIAAKYLIDPEACFLA | 243 |
| | | ++ \| \|+\|+\| \|\| \| \|\|\| \|\|\|\| \| \|\| \| ++\|\|+ + \| \|\| +\|\|+ \| | |
| Pfam01459: | 174 | DYSLHLNLNNGQEFTASYYHKVNSKLETGVKATWNSGTSNNTNIEFATKYRLDPDTSVKA | 233 |
| NOV6a: | 244 | KVNNSSLIGLGYTQTLKPGIKLTLSALLDGKNVNAGGHKLGLGLEFQA | 291 |
| | | \|\|\|\|\| \| \| \| \|+\|\|+ \|\|+\|\| \| \| ++\|\|\|\|\|+\|\| \|\|\|+ | |
| Pfam01459: | 234 | KVNNSGLASLAYQHELRPGVTLTVSASFDTKALDAGGHKVGLSLEFKP | 281 |

Patp BLAST results for NOV6a include those listed in Table 61.

TABLE 6I
Patp alignments of NOV6a

| | | | Smallest |
|---|---|---|---|
| | | | Sum |
| Sequences producing High-scoring- | Reading | High | Prob. |
| Segment Pairs: | Frame | Score | P (N) |
| patp:AAY45015 Protein encoded by | +3 | 931 | 1.2e−92 |
| fchd545 gene [Homo sap], 283 aa | | | |
| patp:AAW36004 Human Fchd545 gene | | | |
| product [Homo sap], 283 aa | | | |
| patp:AAY07222 Voltage-dependent anion | | | |
| channel CBMAAD07 protein sequence | | | |
| [Homo sap], 283 aa | | | |

Patp BLAST results for NOV6b include those listed in Table 6J.

TABLE 6J
Patp alignments of NOV6b

| | | | Smallest |
|---|---|---|---|
| | | | Sum |
| Sequences producing High-scoring- | Reading | High | Prob. |
| Segment Pairs: | Frame | Score | P (N) |
| patp:AAY45015 Protein encoded by- | +2 | 1329 | 7.8e−135 |
| fchd545 gene [Homo sap], 283 aa | | | |
| patp:AAW48908 Human high voltage- | | | |
| dependent anion channel protein- | | | |
| [Homo sap], 283 aa | | | |
| patp:AAW36004 Human Fchd545 gene- | | | |
| product [Homo sap], 283 aa | | | |
| patp:AAY07222 Voltage-dependent anion- | | | |
| channel CBMAAD07 protein- | | | |
| sequence [Homo sap], 283 aa | | | |

The homologies shown above are shared by NOV6b insofar as NOV6b is homologous to NOV6a as shown in Table 6E.

Potassium channels represent the most complex class of voltage-gated ion channels from both functional and structural standpoints. Present in all eukaryotic cells, their diverse functions include maintaining membrane potential, regulating cell volume, and modulating electrical excitability in neurons. The delayed rectifier function of potassium channels allows nerve cells to efficiently repolarize following an action potential. In Drosophila, 4 sequence-related K+ channel genes—Shaker, Shaw, Shab, and Shal—have been identified. Each has been shown to have a human homolog. (Adelman et al., Neuron 15(6): 1449-54, 1995)

By PCR of genomic DNA with primers based on regions conserved between Drosophila Shaker and a mouse voltage-gated potassium channel, Ramaswami et al. (1990) isolated fragments of several related human genes. They used the fragments to screen cDNA libraries and cloned cDNAs encoding several potassium channels that they designated HuKI (KCNA1), HuKII (KCNA4; OMIM:176266), HuKIV (KCNA2; OMIM:176262), and HuKV (KCNA6; OMIM: 176257). Like other Shaker-class potassium channels, the predicted 495-amino acid KCNA1 protein contains 6 hydrophobic segments, a positively charged region called S4 between hydrophobic segments 3 and 4, and a leucine zipper. KCNA1 shares 98% amino acid identity with its rat homolog, RCK1. When expressed in Xenopus oocytes, KCNA1, KCNA4, and KCNA2 exhibited different voltage dependence, kinetics, and sensitivity to pharmacologic potassium channel blockers. KCNA1 and KCNA2 were noninactivating channels and resembled delayed rectifiers, while KCNA4 was rapidly inactivating.

Chandy et al. demonstrated that 3 closely related potassium channel genes, MK1, MK2, and MK3, are located at separate sites in the genome of the mouse. (Chandy et al., Science 247(4945):973-5, 1990) These genes, encoding subunits of voltage-dependent K+ channels, are homologous to the Drosophila Shaker gene. McPherson et al. (1991) mapped member 1 of the Shaker-related subfamily of K+ channel genes (the homolog of MK1) to human chromosome 12 by study of somatic cell hybrids. Curran et al. mapped the KCNA1 gene to chromosome 12 by use of human-rodent somatic cell panels and narrowed the localization to the distal short arm by in situ hybridization (Curran et al., Genomics 12(4):729-37, 1992). Linkage studies had shown a maximum lod score of 2.72 at a recombination fraction of 0.05 between KCNA1 and the von Willebrand locus (VWF; OMIM: 193400). Using interspecific backcrosses between Mus musculus and Mus spretus, Klocke et al. mapped the Kcna1, Kcna5 (OMIM: 176267), and Kcna6 genes to mouse chromosome 6, close to the homolog of TPI1 (OMIM:190450), which is located on 12p13 in the human. (Klocke et al., Genomics 18(3):568-74, 1993) Albrecht et al. determined that a 300-kb cluster on chromosome 12p13 contains the human KCNA6, KCNA1, and KCNA5 genes arranged in tandem. (Albrecht et al., Receptors Channels 3(3):213-20, 1995)

Browne et al. performed mutation analysis of the KCNA1 coding region in 4 families with myokymia (rippling of muscles) with episodic ataxia, also known as episodic ataxia type 1 (EA1; OMIM: 160120). They found 4 different missense mutations present in heterozygous state. (Browne et al., Nat Genet 8(2): 136-40, 1994) For a comprehensive review of episodic ataxia type 1 and its causative mutations, see Brandt and Strupp (Audiol Neurootol 2(6):373-83, 1997). Adelman et al. (1995) injected Xenopus oocytes with cDNAs corresponding to 6 different mutations associated with autosomal dominant myokymia with episodic ataxia. (Adelman et al., Neuron 15(6):1449-54, 1995) They demonstrated that coassembly of one or more episodic ataxia subunits with a wild type subunit can alter channel function, giving a dominant-negative effect.

The above defined information for this invention suggests that these Voltage-Dependent Anion Channel-like proteins (NOV6) may function as a member of a “Voltage-Dependent Anion Channel family”. Therefore, the NOV6 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV6 nucleic acids and proteins of the invention are useful in potential therapeutic applications implicated in in Episodic Ataxia, type 1, Long QT Syndrome 1 and 2, Benign Neonatal Epilepsy, Jervell and Lange-Neilson syndrome, Autosomal dominant deafness (DFNA 2), non-insulin dependent diabetes mellitus, CNS disorders, arrhythmia, seizure, asthma, hypertension therapy and/or other pathologies and disorders. For example, a cDNA encoding the Voltage-Dependent Anion Channel-like protein may be useful in gene therapy, and the Voltage-Dependent Anion Channel-like protein may be useful when administered to a subject in need thereof. By way of nonlimiting example, the compositions of the present invention will have efficacy for treatment of patients suffering from in Episodic Ataxia, type 1, Long QT Syndrome 1 and 2, Benign Neonatal Epilepsy, Jervell and Lange-Neilson syndrome, Autosomal dominant deafness (DFNA 2), non-insulin dependent diabetes mellitus, CNS disorders, arrhythmia, seizure, asthma, hypertension therapy. The NOV6 nucleic acid encoding Voltage-Dependent Anion Channel-like protein, and the Voltage-Dependent Anion Channel-like protein of the invention, or fragments thereof, may further be useful in diagnostic applications, wherein the presence or amount of the nucleic acid or the protein are to be assessed.

NOV6 nucleic acids and polypeptides are further useful in the generation of antibodies that bind immuno-specifically to the novel NOV6 substances for use in therapeutic or diagnostic methods. These antibodies may be generated according to methods known in the art, using prediction from hydrophobicity charts, as described in the “Anti-NOVX Antibodies” section below. For example the disclosed NOV6 proteins have multiple hydrophilic regions, each of which can be used as an immunogen. In one embodiment, a contemplated NOV6a epitope is from about amino acids 10 to 20. In another embodiment, a NOV6b epitope is from about amino acids 10 to 85. In additional embodiments, NOV6a epitopes are from about amino acids 35 to 85, from about amino acids 95 to 140, and from about amino acids 150 to 230. In further embodiments, NOV6b epitopes are from about amino acids 95 to 130, from about amino acids 145 to 195, and from about amino acids 210 to 240. This novel protein also has value in development of powerful assay system for functional analysis of various human disorders, which will help in understanding of pathology of the disease and development of new drug targets for various disorders.

NOV7

A disclosed NOV7 nucleic acid of 1494 nucleotides (also referred to AC016572 da 1) encoding a novel butyrophilin-like receptor protein is shown in Table 7A. An open reading frame was identified beginning with a ATG initiation codon at nucleotides 1-3 and ending with a TGA codon at nucleotides 1492-1494. In Table 7A, and the start and stop codons are in bold letters.

TABLE 7A
NOV7 Nucleotide Sequence

| | |
|---|---|
| ATGAACCCGGTACCTCAGATGGAAATGCAGAAATCACCCATGTTCTGCGTCGCTCACTCTGGGA | (SEQ ID NO:15) |
| GCTGTAGACCGGAGCTGTTCCTATTCGGCCATCTTGGCTCCTCCCCCTCACTACAAAGGGTTTT | |
| GATGATTTATCTTTTGTATGTCTTCCCACAGGGCAGTGGCAGGTGTTTGGGCCAGACAAGCCTG | |
| TCCAGGCCTTGGTGGGGGAGGACGCAGCATTCTCCTGTTTCCTGTCTCCTAAAACCAATGCAGA | |
| GGCCATGGAAGTGCGGTTCTTCAGGGGCCAGTTCTCTAGCGTGGTCCACCTCTACAGGGACGGG | |
| AAGGACCAGCCATTTATGCAGATGCCACAGTATCAAGGCAGGACAAAACTGTGAAGGATTCTAT | |
| TGCGGAGGGGCGCATCTCTCGAGGCTGGAAAACATTACTGTGTTGGATGCTGGCCTCTATGGGT | |
| GCAGGATTAGTTCCCAGTCTTACTACCAGAAGGCCATCTGGGAGCTACAGGTGTCAGCACTGGG | |
| CTCAGTTCCTCTCATTTCCATCACGGGATATGTTGATAGAGACATCCAGCTACTCTGTCAGTCC | |
| TCGGGCTGGTTCCCCCGGCCCGTGCAAGGAGCCAGCGTCGTGTTTGTGCCTTGTACACTCCTGT | |
| GTCCACCACTGAATATACTGTTTCTGTTTCAGGGAAAATCCAGGCGGAACTGGGTAAGTATGTG | |
| TCATGTCCTGAGCCTCCCACACATGGTTCTCCCGGGTCCCTCCCTGATCCACAGTTTGAGCCTC | |
| TGGACGACCCTGGCTGCAGGCTGGACAGGAAGCACCGACTGGAGAAGAAAGCACGGACAGGCAG | |
| AATTGAGAGACGCCCGGAAACACGCAGTGGAGGTGACTCTGGATCCAGAGACGGCTCACCCGAA | |
| GCTCTGCGTTTCTGATCTGAAAACTGTAACCCATAGAAAAGCTCCCCAGGAGGTGCCTCACTCT | |
| GAGAAGAGATTTACAAGGAAGAGTGTGGTGGCTTCTCAGAGTTTCCAAGCAGGGAAACATTACT | |
| GGGAGGTGGACGGAGGACACAATAAAAGGTGGCGCGTGGGAGTGTGCCGGGATGATGTGGACAG | |
| GAGGAAGGAGTACGTGACTTTGTCTCCCGATCATGGGTACTGGGTCCTCAGACTGAATGGAGAA | |
| CATTTGTATTTCACATTAAATCCCCGTTTTATCAGCGTCTTCCCCAGGACCCCACCTACAAAAA | |
| TAGGGGTCTTCCTGGACTATGAGTGTGGGACCATCTCCTTCTTCAACATAAATGACCAGTCCCT | |
| TATTTATACCCTGACATGTCGGTTTGAAGGCTTATTGAGGCCCTACATTGAGTATCCGTCCTAT | |
| AATGAGCAAAATGGAACTCCCATAGTCATCTGCCCAGTCACCCAGGAATCAGAGAAAGAGGCCT | |
| CTTGGCAAAGGGCCTCTGCAATCCCAGAGACAAGCAACAGTGAAGTCCTCCTCACAGGCAACCA | |
| CGCCCTTCCTCCCCAGGGGTGA | |

The disclosed NOV7 nucleic acid sequence has 721/821 (87%) identical to a butyrophilin like receptor mRNA from Homo Sapiens (GENBANK-ID: AB020625|acc:AB020625 (E=3.5e⁻¹⁹⁰).

A disclosed NOV7 polypeptide (SEQ ID NO:16) encoded by SEQ ID NO:15 is 497 amino acid residues and is presented using the one-letter amino acid code in Table 7B. Signal P, Psort and/or Hydropathy results predict that NOV7 has a signal peptide and is likely to be localized in the nucleus with a certainty of 0.8700, the plasma membrane with a certainty of 0.7000 and the microbody (peroxisome) with a certainty of 0.6171. The NOV7 protein predicted here is similar to the butyrophilin-like receptor protein family, some members of which have presented at the plasma membrane. Therefore it is likely that this NOV7 protein is available at the same sub-cellular localization and hence accessible to a diagnostic probe and for various therapeutic applications. The most likely cleavage site for a NOV7 peptide is between amino acids 56 and 57, at: GSG-RC.

TABLE 7B
Encoded NOV7 protein sequence.

| | |
|---|---|
| MNPVPQMEMQKSPMFCVAHSGSCRPELFLFGHLGSSPSLQRVLMIYLLYVFPQGSGRCLGQTSL | (SEQ ID NO:16) |
| SRPWWGRTQHSPVSCLLKPMQRPWKCGSSGASSLAWSTSTGTGRTSHLCRCHSIKAGQNCEGFY | |
| CGGAHLSRLENITVLDAGLYGCRISSQSYYQKAIWELQVSALGSVPLISITGYVDRDIQLLCQS | |
| SGWFPRPVQGASVVFVPCTLLCPPLNILFLFQGKSRRNWVSMCHVLSLPHMVLPGPSLIHSLSL | |
| WTTLAAGWTGSTDWRRKHGQAELRDARKHAVEVTLDPETAHPKLCVSDLKTVTHRKAPQEVPHS | |
| EKRFTRKSVVASQSFQAGKHYWEVDGGHNKRWRVGVCRDDVDRRKEYVTLSPDHGYWVLRLNGE | |
| HLYFTLNPRFISVFPRTPPTKIGVFLDYECGTISFFNINDQSLIYTLTCRFEGLLRPYIEYPSY | |
| NEQNGTPIVICPVTQESEKEASWQRASAIPETSNSEVLLTGNHALPPQG | |

NOV7 maps to chromosome 5p35 and was found to be expressed in at least the following tissues: mammary gland, small intestine, colon, testis and leukocytes.

The disclosed NOV7 amino acid sequence has 249 of 337 amino acid residues (73%) identical to, and 277 of 337 amino acid residues (82%) similar to, the Homo sapiens 432 amino acid residue BUTYROPHILIN LIKE RECEPTOR(SPTREMBL-ACC:Q9Y2C7)(2.1e⁻¹²⁹).

NOV7 also has homology to the amino acid sequence shown in the BLASTP data listed in Table 7C.

TABLE 7C
BLAST results for NOV7

| Gene Index/ | Protein/ | Length | Identity | Positives | |
|---|---|---|---|---|---|
| Identifier | Organism | (aa) | (%) | (%) | Expect |
| gi\|5729748\|ref\|NP_ | butyrophilin- | 432 | 246/338 | 273/338 | 1e−135 |
| 006698.1\| | like 3; | | (72%) | (79%) | |
| | butyrophilin- | | | | |
| | like receptor | | | | |
| | [Homo sapiens] | | | | |

The homology of these sequences is shown graphically in the ClustalW analysis shown in Table 7D.

[image]

[image]

Table 7E and 7F lists the domain description from DOMAIN analysis results against NOV7. This indicates that the NOV7 sequence has properties similar to those of other proteins known to contain this domain.

TABLE 7E
Domain Analysis of NOV7

gnh|Smart|smart00449, SPRY, Domain in SPla and the RYanodine
Receptor.; Domain of unknown function. Distant homologues are
domains in butyrophilin/marenostrin/pyrin homologues. (SEQ ID NO:63)
Length = 125 residues, 97.6% aligned
Score = 76.3 bits (186), Expect = 4e−15

| | | | |
|---|---|---|---|
| NOV7: | 338 | GKHYWEV--DGGHNKRWRVGVCRDDVDRRKEYVTLSPDHGYWVLRLNGEHLYFTLNPRFI | 395 |
| | | \|+\|\|+\|\| \| +\| \| + \| \| \| \| +\| \| | |
| smart00449: | 62 | GRHYFEVEVFTGGDKGHWRVGWATKSVPRGGFRLLGEDKGSWGYDGDGGKKYHNSEFPEY | 61 |
| NOV7: | 396 | SVFPRTPPTKIGVFLDYECGTISFFNINDQSLIYTLTCRFEGLLRPYIEYPSYNEQNGTP | 455 |
| | | + + \| \|\| \|\|\| \| \|\|\|\|\|+ + \| \| \| \| + \| | |
| smart00449: | 62 | GLPFQEPGDVIGCFLDLEAGTISFYKNGKYLGLAFFDVTFSGPLYPAV---SLGNGGSVR | 118 |
| NOV7: | 456 | IVICP | 460 |
| | | + \| | |
| smart00449: | 119 | LNFGP | 123 |

TABLE 7F
Domain Analysis of NOV7

gnl|Pfam|pfam00622, SPRY, SPRY domain. (SEQ ID NO:64)
Length = 123 residues, 97.6% aligned
Score = 67.0 bits (162), Expect = 2e−12

| | | | |
|---|---|---|---|
| NOV7: | 338 | GKHYWEVDGGHNKRWRVGVCRDDVDRRKEYVTLSPD-HGYWVLRLNGEHLYFTLNPRFIS | 396 |
| | | \|\|\|\|+\|\|+ + \|\| 10 +\| \| \| \| \| \| | |
| pfam00622: | 2 | GKHYFEVEVDTGGETHWRIGWATKSVRKPGESLLGDNEGSWGFDGTGGKKYHNGFGE-DY | 60 |
| NOV7: | 397 | VFPRTPPTKIGVFLDYECGTISFFNINDQSLIYTLT-CRFEGLLRPYIEYPSYNEQNGTP | 455 |
| | | \| \|\| \|\|\| \| \| \|\|\| \| + \| \| \| \| \| + \| | |
| pfam00622: | 61 | GLPFQEGDVIGCFLDLESGEISFTK-NGKYLGVAFRNVTFGGPLYPAV---SLGSGEAVQ | 116 |
| NOV7: | 456 | IVICP | 460 |
| | | + \| | |
| pfam00622: | 117 | LNFGP | 121 |

Patp BLAST results for NOV7 include those listed in Table 7G.

TABLE 7G
Patp alignments of NOV7

| | | | Smallest |
|---|---|---|---|
| | | | Sum |
| Sequences producing High-scoring | Reading | High | Prob. |
| Segment Pairs: | Frame | Score | P (N) |
| patp:AAB87567 Human PRO1347 | −1 | 1178 | 2.4e−197 |
| [Homo sap], 500 aa | | | |
| patp:AAY99385 Human PRO1347 | | | |
| (UNQ702) amino acid sequence | | | |
| SEQ ID NO:148 [Homo sap], 500 aa | | | |
| patp:AAB66134 Protein of the | | | |
| invention #46 [Homo sap], 500 aa | | | |

Shibui et.al. isolated a cDNA clone which shows a similarity with human butyrophilin from a human colon mucosa cDNA library (J Hum Genet 44(4):249-52, 1999). The cDNA is 1964 bases long, with one open reading frame encoding a protein of 433 amino acids. The deduced amino acid sequence shows an overall homology of 36.5% with the human butyrophilin protein. This gene is mainly expressed in small intestine, colon, testis, and leukocytes. The chromosomal location of the gene was determined on the chromosome 5q35 region by polymerase chain reaction-based analysis with both a human/rodent monochromosomal hybrid cell panel and a radiation hybrid mapping panel. (Shibui et al., J Hum Genet 44(4):249-52, 1999).

Human butyrophilin was cloned and sequenced from a human breast cDNA library. The derived amino acid sequence shows 84% sequence identity and identical domain arrangements with the previously reported bovine sequence. Sequence analysis reveals an immunoglobulin constant (IgC) domain that was not previously identified in the bovine sequence. The extracellular domain composition of butyrophilin suggests a cell surface receptor function. (Taylor et.al., Biochim Biophys Acta 1306(1):1-4, 1996)

The molecular and cellular biology of the milk protein butyrophilin was reviewed by Mather et.al. (J Dairy Sci 76(12):3832-50, 1993). Butyrophilin constitutes more than 40% by weight of the total protein associated with the fat globule membrane of bovine milk. Closely related proteins are abundant in the fat globule membranes of many other species. Butyrophilin is synthesized as a peptide of 526 amino acids with an amino-terminal hydrophobic signal sequence of 26 amino acids, which is cleaved before secretion in association with the fat globule membrane. Hydropathy analysis and in vitro translation of butyrophilin mRNA indicate that the protein associates with membranes in a type I orientation via a single stretch of 27 hydrophobic amino acids in the approximate middle of the sequence. Evidence that butyrophilin is incorporated into fat globule membrane as a transmembrane protein and as a cytoplasmically oriented peripheral component is discussed. The carboxy-terminal sequence of butyrophilin is significantly homologous to two other proteins: ret finger protein and the 52-kDa nuclear antigen A of Sjogren's syndrome. Expression of bovine butyrophilin mRNA correlates with the onset of milk fat secretion toward the end of pregnancy and is maintained throughout lactation. The possible function of butyrophilin in the secretion of milk lipid droplets is also discussed. (Mather et.al., J Dairy Sci 76(12);3832-50, 1993)

The protein similarity information, expression pattern, and map location for the butyrophilin-like receptor protein and nucleic acid (NOV7) disclosed herein suggest that NOV7 may have important structural and/or physiological functions characteristic of the butyrophilin-like receptor family. Therefore, the NOV7 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV7 nucleic acids and proteins of the invention are useful in potential diagnostic and therapeutic applications implicated in various diseases and disorders described below and/or other pathologies. For example, the compositions of the present invention will have efficacy for treatment of patients suffering from Fertility, Inflammatory bowel disease, Diverticular disease, Autoimmune disorders and Cancer. The NOV7 nucleic acid, or fragments thereof, may further be useful in diagnostic applications, wherein the presence or amount of the nucleic acid or the protein are to be assessed.

NOV7 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV7 protein has multiple hydrophilic regions, each of which can be used as an immunogen. This novel protein also has value in development of powerful assay system for functional analysis of various human disorders, which will help in understanding of pathology of the disease and development of new drug targets for various disorders.

NOV8

A disclosed NOV8 nucleic acid of 3065 nucleotides (also referred to as 101360122_EXT4) encoding a novel MEGF/FIBRILLIN-like protein is shown in Table 8A. An open reading frame was identified beginning with a ATG initiation codon at nucleotides 16-18 and ending with a TAG codon at nucleotides 3034-3036. A putative untranslated region upstream from the initiation codon and downstream from the termination codon is underlined in Table 8A, and the start and stop codons are in bold letters.

TABLE 8A
NOV8 Nucleotide Sequence

| | |
|---|---|
| CCAGTAGCTCCAGCCATGGGCTCGGGGCGCGTACCCGGGCTCTGCCTGCTTGTCCTGCTGGTCC | (SEQ ID NO:17) |
| ACGCCCGCGCCGCCCAGTACAGCAAAGCCGCGCAAGGTAAGGAAGGAGGGGCGCGCGGCCTGGG | |
| GGCTGTCCTGGCTGCTGGGCCTCAGGGCCTAGGAGCGATTCCCGAGGGGCAGGGCAGGTGCTGG | |
| GGACCCCTGCCAGATGTGGATGAGTGTGTGGAGGGGACTGACAACTGCCACATCGATGCTATCT | |
| GCCAGAACACCCCGAGGTCATACAAGTGCATCTGCAAGTCTGGCTACACACGGGACGGCAAACA | |
| CTGCAAAGACGTGGATGAGTGCGAGCGAGAGGATAATGCAGGTTGTGTGCATGACTGTGTCAAC | |
| ATCCCTGGCAATTACCGGTGTACCTGCTATGATGGATTCCACCTGGCACATGACGGACACAACT | |
| GTCTGGATGTGGACGAGTGTGCCGAGGGCAACGGCGGCTGTCAGCAGAGCTGTGTCAACATGAT | |
| GGGCAGCTATGAGTGCCACTGCCGGGAAGGCTTCTTCCTCAGCGACAACCAGCATACCTGTATC | |
| CAGCGGCCAGAAGAAGGAATGAATTGCATGAACAAGAACCACGGCTGTGCCCACATTTGCCGGG | |
| AGACACCCAAGGGGGGTATTGCCTGTGAATGCCGTCCTGGCTTTGAGCTTACCAAGAACCAACG | |
| GGACTCTAAATTGACATGCAACTATGGTAACGGCGGCTGCCAGCACACGTGTGATGACACAGAG | |
| CAGGGTCCCCGGTCCGGCTGCCATATCAACTTTGTGCTCCATACCGACGGGAAGACATGCATCG | |
| GGGAAAGGCGGCTAGAGCAGCACATCCCCACTCAAGCCGTTTCTAATGAGACCTGTGCTGTCAA | |
| CAACGGGGGCTGTGACAGTAAGTGCCATGATGCAGCGACTGGTGTCCACTGCACCTGCCCTGTG | |
| GGCTTCATGCTGCAGCCAGACAGGAAGACGTGCAAAGATATAGATGAGTGCCGCTTAAACAACG | |
| GGGGCTGTGACCATATTTGCCGCIACACAGTGGGCAGCTTCGAATGCAGTTGCAAGAAAGGCTA | |
| TAAGCTTCTCATCAATGAGAGGAACTGCCAGGATATAGACGAGTGTTCCTTTGATCGAACCTGT | |
| GACCACATATGTGTCAACACACCAGGAAGCTTCCAGTGTCTCTGCCATCGTGGCTACCTGTTGT | |
| ATGGTATCACCCACTGTGGGGATGTGCATGAATGCAGCATCAACCGGGGAGGTTGCCGCTTTGG | |
| CTGCATCAACACTCCTGGCAGCTACCAGTGTACCTGCCCAGCAGGCCAGGGTCGGCTGCACTGG | |
| AATGGCAAAGATTGCACAGAGCCACTGAAGTGTCAGGGCAGTCCTGGGGCCTCGAAAGCCATGC | |
| TCAGCTGCAACCGGTCTGGCAAGAAGGACACCTGTGCCCTGACCTGTCCCTCCACCATCCCTCT | |
| AGAGGCTGCAGTGCTGTCCATTAAACAACGGGCCTCCTTCAAGATCAAGGATGCCAAATCCCGT | |
| TTGGCTGGGAGTGAGAACTTTCCAGGGGATGTCCCTGGGGTTGACAAGCCCTCTCTCCCAGGTG | |
| GTGCCCCCTGCTCTGAATGCCAGGTCACCTTCATCCACCTTAAGTGTGACTCCTCTCGGAAGGG | |
| CAAGGGCCGACGGGCCCGGCCCTCCTGCTCACAGCAACTCTTTCTCCTCCCTGATACACACGGC | |
| CATCCACCACCAGCCAGCTGTGGGCTGCCCTGCCTCCGACAGCGAATGGAACGGCGGCTGAAAG | |
| GATCCCTGAAGATGCTCAGAAAGTCCATCAACCAGGACCGCTTCCTGCTGCGCCTGGCAGGCCT | |
| TGATTATGAGCTGGCCCACAAGCCGGGCCTGGTAGCCGGGGAGCGAGCAGAGCCGATGGAGTCC | |
| TGTAGGCCCGGGCAGCACCGTGCCGACTCTCCCTCAGTCAGCTGCCCGCAGGGAACGTATTACC | |
| ACGGCCAGACGGAGCAGTGTGTGCCATGCCCAGCGGGCACCTTCCAGGAGAGAGAAGGGCAGCT | |
| CTCCTGCGACCTTTGCCCGAGAGAGAAAGACCAAGGATCTCCTAAGCCCCGGAAGCACGGCGCG | |
| GGACCCAAGTGTGGTCAGCGCCAACGGCGCAAACATTCGGAAGATGGGTTCAAGCCCTGTCAGC | |
| CATGCCCACGTGGCACCTACCAACCTGAAGCAGGACGGACCCTATGCTTCCCTTGTGGTGGGGG | |
| CCAGGGCACTACTACAACACCACCATCCACCGCTGTATTCGCTGTGCCATGGGCTCCTATCAGC | |
| CCGACTTCCGTCAGAACTTCTGCAGCCGCTGTCCAGGAAACACAAGCACAGACTTTGATGGCTC | |
| TACCAGTGTGGCCCAATGCAAGAATCGTCAGTGTGGTGGGGAGCTGGGTGAGTTCACTGGCTAT | |
| ATTGAGTCCCCCAACTACCCGGGCAACTACCCAGCTGGTGTGGAGTGCATCTGGAACATCAACC | |
| CCCCACCCAAGCGCAAGATCCTTATCGTGGTACCAGAGATCTTCCTGCCATCTGAGGATGAGTG | |
| TGGGGACGTCCTCGTCATGAGAAAGAACTCATCCCCATCCTCCATTACCACTTATGACACCTGC | |
| CAGACCTACGAGCGTCCCATTGCCTTCACTGCCCGTTCCAGGAAGCTCTGGATCAACTTCAAGA | |
| CAAGCGAGGCCAACAGCGCCCGTGGCTTCCAGATTCCCTATGTTACCTATGATGAGGACTATGA | |
| GCAGCTGGTAGAAGACATTGTGCGAGATGGCCGGCTCTATGCCTCTGAAAACCACCAGGAGATT | |
| TTAAAGGACAAGAAGCTCATCAAGGCCTTCTTTGAGGTGCTAGCCCACCCCCAGAACTACTTCA | |
| AGTACACAGAGAAACACAAGGAGATGCTGCCAAAATCCTTCATCAAGCTGCTCCGCTCCAAAGT | |
| TTCCAGCTTCCTGAGGCCCTACAAATAGTAACCCTAGGCTCACACACGCCAACGCGT | |

The disclosed NOV8 nucleic acid sequence has similarity to several fragments of the sequence of fibrillin-2 mRNA from mouse (GENBANK-ID:MUSFBN2|acc:L39790), including a fragment having 236 of 374 bases (63%) identical to a fibrillin-2 mRNA from mouse (E=3.7e²¹).

A disclosed NOV8 protein (SEQ ID NO:18) encoded by SEQ ID NO:17 has 1006 amino acid residues, and is presented using the one-letter code in Table 8B. Signal P, Psort and/or Hydropathy results predict that NOV8 has a signal peptide and is likely to be localized extracellularly with a certainty of 0.3700. The sequence has three hydrophobic regions apart from the region spanning the putative signal peptide, which could constitute a hydrophobic core of the protein. The most likely cleavage site for a NOV8 peptide is between amino acids 21 and 22, at: RAA-QY. NOV8 has a molecular weight of 110709.2 Daltons.

TABLE 8B
Encoded NOV8 protein sequence.

| | |
|---|---|
| MGSGRVPGLCLLVLLVHARAAQYSKAAQGKEGGARGLGAVLAACPQGLGAIPEGQGRCWGPLPD | (SEQ ID NO:18) |
| VDECVEGTDNCHIDAICQNTPRSYKCICKSGYTGDGKHCKDVDECEREDNAGCVHDCVNIPGNY | |
| RCTCYDGFHLAHDCHNCLDVDECAECNGGCQQSCVNMMOSYECHCREGFFLSDNQHTCIQRPEE | |
| GMNCMNKNHGCAHICRETPKGGIACECRPGFELTKNQRDCKLTCNYGNGGCQHTCDDTEQGPRC | |
| GCHIKFVLHTDGKTCIGERRLEQHIPTQAVSNETCAVNNGGCDSKCHDAATGVHCTCPVGFMLQ | |
| PDRKTCKDIDECRLNNGGCDHICRNTVGSFECSCKKGYKLLINERNCQDTDECSFDRTCDHICV | |
| NTPGSFQCLCHRGYLLYGITHCGDVDECSINRGGCRFGCINTPGSYQCTCPAGQGRLHWNGKDC | |
| TEPLKCQGSPGASKAMLSCNRSGKKDTCALTCPSTIPLEAAVLSIKQRASFKIKDAKCRLAGSE | |
| NFPGDVPGVDKPSLPGGAPCSECQVTFIHLKCDSSRKGKGRRARPSCSQQLFLLPDTHGHPPPA | |
| SCGLPCLRQRMERRLKGSLKMLRKSINQDRFLLRLAGLDYELAHKPGLVAGERAEPMESCRPGQ | |
| HRADSPSVSCPQGTYYHGQTEQCVPCPAGTFQEREGQLSCDLCPREKDQGSPKPRKHGAGPKCG | |
| QRQRRKHSEDGFKPCQPCPRGTYQPEAGRTLCFPCGGGLTTKHEGAISFQDCDTKVQCSPGHYY | |
| NTSIHRCIRCAMGSYQPDFRQNFCSRCPGNTSTDFDGSTSVAQCKNRQCGGELGEFTGYIESPN | |
| YPGNYPAGVECIWNINPPPKRKILIVVPEIFLPSEDECGDVLVMRKNSSPSSITTYETCQTYER | |
| PIAFTARSRKLWINFKTSEANSARGFQIPYVTYDEDYEQLVEDIVRDGRLYASENHQEILKDKK | |
| LIKAFFEVLAHPQNYFKYTEKHKEMLPKSFIKLLRSKVSSFLRPYK | |

NOV8 maps to chromosome 6 and was found to be expressed in at least the following tissues: kidney.

The disclosed NOV8 amino acid has 606 of 996 amino acid residues (60%) identical to, and 751 of 996 amino acid residues (75%) similar to, the 999 amino acid residue CEGP1 protein from human (TREMBLNEW-ACC:CAB92285)(E=0.0).

NOV8 also has homology to the amino acid sequences shown in the BLASTP data listed in Table 8C.

TABLE 8C
BLAST results for NOV8

| Gene Index/ | | Length | Identity | Positives | |
|---|---|---|---|---|---|
| Identifier | Protein/Organism | (aa) | (%) | (%) | Expect |
| gi\|10190748\|ref\|NP_ | CEGP1 protein | 999 | 595/983 | 741/983 | 0.0 |
| 066025.1\| | [Homo sapiens] | | (60%) | (74%) | |
| gi\|9910154\|ref\|NP_ | Cegp1 protein; | 997 | 579/1007 | 732/1007 | 0.0 |
| 064436.1\| | ICRFP703B1614Q5.1; | | (57%) | (72%) | |
| | ICRFP703N2430Q5.1 | | | | |
| | [Mus musculus] | | | | |
| gi\|12738840\|ref\|NP_ | signal peptide, | 961 | 548/912 | 650/912 | 1e−130 |
| 073560.1\| | CUB domain, EGF- | | (60%) | (71%) | |
| | like 1 [Mus | | | | |
| | musculus] | | | | |

The homology of these sequences is shown graphically in the ClustalW analysis shown in Table 8D.

[image]

[image]

[image]

[image]

Tables 8E-8H list the domain description from DOMAIN analysis results against NOV8. This indicates that the NOV8 sequence has properties similar to those of other proteins known to contain this domain.

TABLE 8E
Domain Analysis of NOV8

gnl|Smart|smart00042, CUB, Domain first found in Clr, Cls,
uEGF, and bone morphogenetic protein.; This domain is found
mostly among developmentally-regulated proteins. Spermadhesins
contain only this domain. (SEQ ID NO:68)
Length = 114 residues, 99.1% aligned
Score = 80.5 bits (197), Expect = 4e−16

| | | | |
|---|---|---|---|
| NOV8: | 817 | CGGELGEFTGYIESPNYPGNYPAGVECIWNINPPPKRKILIVVPEIFLPSEDECG-DVLV | 875 |
| | | \|\|\| \| +\| \| \|\|\|\|\| +\|\| + \|+\| \|+ \|\| +\| + + \| \| \| \| \| + | |
| smart00042: | 1 | CGGTLTASSGTITSPNYPNSYPNNLNCVWTISAPPGYRIELKFTDFDLESSDNCTYDYVE | 60 |
| NOV8: | 876 | MRKNSSPSSITTYETCQTYERPIAFTARSRKLWINFKTSEANSARGFQIPYVT | 928 |
| | | + \| \|\| \| + \| ++ \| + + \| + + \|\|\| \| | |
| smart00042: | 61 | IYDGPSTSSPLLGRFCGSELPPPIISSSSNSMTVTFVSDSSVQKRGFSARYSA | 113 |

TABLE 8F
Domain Analysis of NOV8

gnl|Pfam|pfam00431, CUB, CUB domain. (SEQ ID NO:69)
Length = 110 residues, 100.0% aligned
Score = 80.5 bits (197), Expect = 4e−16

| | | | |
|---|---|---|---|
| NOV8: | 817 | CGGELGEFTGYIESPNYPGNYPAGVECIWNINPPPKRKILIVVPEIFLPSEDECG-DVLV | 875 |
| | | \|\|\| \| \| +\| \| \|\|\|\|\| \|\| \|\|+\| \| \|\| ++ + + \| \| \|\| \| + | |
| pfam00431: | 1 | CGGVLTESSGSISSPNYPNPYPPNKECVWRIRAPPGYRVSLTFQDFDLEDHDGCGYDYVE | 60 |
| NOV8: | 876 | MRKNSSPSSITTYETCQTYERPIAFTARSRKLWINFKTSEANSARGFQIPY | 926 |
| | | +\| \|\| \| \| + \| ++ \| \| + + \|\|\|+ \| | |
| pfam00431: | 61 | IRDGDPSSSPLLGRFC-GSGIPEDIRSSSNRMTIKFVSDASVQKRGFKATY | 110 |

TABLE 8G
Domain Analysis of NOV8

gnl|Smart|smart00179, EGF_CA, Calcium-binding EGF-
like domain SEQ ID NO:70)
Length = 41 residues, 97.6% aligned
Score = 397 bits (91), Expect = 8e−04

| | | | |
|---|---|---|---|
| NOV8: | 64 | DVDECVEGTDNCHIDAICQNTPRSYKC-ICKSGYTGDGKHC | 103 |
| | | \|+\|\|\| \| + \| \| \|\| \|\|+\| \| \|\|\| \|\|++\| | |
| smart00179: | 1 | DIDECASG-NPCQNGGTCVNTVGSYRCEECPPGYTLDGRNC | 40 |

TABLE 8H
Domain Analysis of NOV8

gnl|Smart|smart00179, EGF_CA, Calcium-binding EGF-like domain
(SEQ ID NO:71)
Length = 41 residues, 90.2% aligned
Score = 36.6 bits (83), Expect = 0.007

| | | | |
|---|---|---|---|
| NOV8: | 369 | DIDECSFDRTCDH--ICVNTPGSFQC-LCHRGYLLYG | 402 |
| | | \|\|\|\|\|+ \| + \|\|\|\| \|\|++\| \| \|\| \| \| | |
| smart00179: | 1 | DIDECASGNPCQNGGTCVNTVGSYRCEECPPGYTLDG | 37 |

Patp BLAST results for NOV8 include those listed in Table 81.

TABLE 8I
Patp alignments of NOV8

| | | | Smallest |
|---|---|---|---|
| | | | Sum |
| Sequences producing High-scoring | Reading | High | Prob. |
| Segment Pairs: | Frame | Score | P (N) |
| patp:AAY07735 Breast-specific BS200 | +1 | 1698 | 6.2e−174 |
| protein [Homo sap], 516 aa | | | |
| patp:AAB00192 Breast cancer protein BCO2 | +1 | 1513 | 2.5e−154 |
| [Homo sap], 392 aa | | | |

Possible SNPs found for NOV8 are listed in Table 8J.

TABLE 8J
SNPs

| Consensus | Base | Amino Acid | Amino Acid |
|---|---|---|---|
| Position | Change | Position | Change |
| 2803 | G > C | 930 | D > H |

Proteins belonging to the MEGF/Fibrillin family of proteins share a common feature of having epidermal growth factor (EGF)-like motifs. Examples of this family include the MEGF proteins, which are expressed in the brain and may be involved in neural development and function, and the fibrillins, which are involved in extracellular matrix structure and maintenance. It also includes the latent transforming growth factor beta (TGFbeta) binding proteins, which regulate the release of TGFbeta extracellularly and thereby modulate the involvement of this growth factor in development and disease. However, there are a number of proteins sharing this motif with very diverse cellular roles and the functional significance of the EGF-like domain is unclear. However, defects in these proteins can have profound effects on cellular and extracellular physiology and structure. For example, a mutation in fibrillin 1 causes Marfan syndrome, a disease that involves connective tissue, bone and lung manifestations.

The Marfan syndrome (MFS), initially described just over 100 years ago, was among the first conditions classified as a heritable disorder of connective tissue. MFS lies at one end of a phenotypic continuum, with people in the general population who have one or another of the features of MFS at the other end, and those with a variety of other conditions in between. Diagnosis of MFS and these other conditions remains based on clinical features. Mutations in FBN1, the gene that encodes fibrillin-1, are responsible for MFS and (in a few patients) other disorders in the continuum. In addition to skeletal, ocular, and cardiovascular features, patients with MFS have involvement of the skin, integument, lungs, and muscle tissue. Over the past 30 years, evolution of aggressive medical and surgical management of the cardiovascular problems, especially mitral valve prolapse, aortic dilatation, and aortic dissection, has resulted in considerable improvement in life expectancy. (Pyeritz, Annu Rev Med 51:481-510, 2000)

Fibrillins 1 and 2 are the main constituents of the extracellular microfibrils responsible for the biomechanical properties of most tissues and organs. They are cysteine-rich glycoproteins predominantly made of multiple repeats homologous to the calcium-binding epidermal growth factor module, and are translated as precursor proteins cleaved by furine/PACE-like activities. Fibrillins polymerize extracellularly as parallel bundles of head-to-tail monomers. Binding to calcium rigidifies the structure of the monomers and the supramolecular organization of the macroaggregates. Fibrillin-1 mutations result in the pleiotropic manifestations of Marfan syndrome, and fibrillin-2 alterations cause the overlapping phenotype of congenital contractural arachnodactyly. It is hypothesized that fibrillin-2 guides elastogenesis, whereas fibrillin-1 provides force-bearing structural support. Gene targeting work in the mouse is shedding new light on their distinct and overlapping contributions to tissue morphogenesis and homeostasis. It is also providing an animal model in which to test therapies aimed at reducing hemodynamic stress and the collapse of the aortic matrix during dissecting aneurysm. (Ramirez and Pereira, Int J Biochem Cell Biol 31(2):255-9, 1999)

The latent transforming growth factor-beta binding proteins (LTBP) are a recently identified family of widely expressed multidomain glycoproteins that range in size from 125 kDa to 240 kDa. Four LTBP genes have been described, and the homology of latent transforming growth factor-beta binding proteins molecules to the fibrillins has resulted in their inclusion in the so-called ‘fibrillin superfamily’. They form intracellular covalent complexes with latent transforming growth factor-beta and target these growth factors to the extracellular matrix. This review describes their structure, summarizes current understanding of their dual roles as growth factor binding proteins and components of the extracellular matrix, and highlights their significance in tissue development and disease. (Sinha et al., Matrix Biol 17(8-9):52945, 1998)

Protein engineering studies on human C1r and C1s revealed important characteristics of the individual domains of these multidomain serine-proteases, and supplied evidence about the cooperation of the domains to create binding sites, and to control the activation process. The recombinant subcomponents were expressed in the baculovirus-insect cell system and the biological activity was checked. Deletions and point mutants of C1r were constructed and C1r-C1s chimeras were also produced. Deletion mutants demonstrated that the N-terminal CUB domain and the EGF-like domain of C1r together are responsible for the calcium dependent C1r-C1s interaction. It seems very likely that these two modules form the calcium-binding site of the C1r alpha-fragment and participate in the tetramer formation. The deletion mutants also demonstrated that the N-terminal region of the C1r molecule contains essential elements involved in the control of activation of the serine-protease module. The substrate specificity of the serine-protease is also determined by the five N-terminal noncatalytic domain of C1r/C1s chimera, which contains the catalytic domain of C1s preceded by the N-terminal region of C1r, could replace the C1r in the hemolytically active C1 complex. The C1s/C1r chimera, in which the alpha-fragment of the C1r was replaced for that of the C1s exibits both C1r- and C1s-like characteristics. The zymogen form of human C1r was stabilized by mutating the Arg(463)-Ile(464) bond. Using stable zymogen C1r it was shown that one active C1r in the C1 complex is sufficient for the full activity of the entire complex. Further experiment with this mutant could provide important information about the structure of the C l complex. (Gal and Zavodszky, Immunobiology 199(2):317-26, 1998)

Two consensus domains (CUB and EGF-like) have been shown to be important to members of the MEGF/Fibrillin protein familiy. The CUB domain is an extracellular domain of approximately 110 residues which is found in functionally diverse, mostly developmentally regulated proteins (Bork and Beckmann, J Mol Biol 231:539-545, 1993; Bork, FEBS Lett 282:9-12, 1991). Almost all CUB domains contain four conserved cysteines which probably form two disulfide bridges (C1-C2, C3-C4). The structure of the CUB domain has been predicted to be a beta-barrel similar to that of immunoglobulins. Proteins that have been found to contain the CUB domain include mammalian complement subcomponents C1s/C1r, which form the calcium-dependent complex C1, the firstcomponent of the classical pathway of the complement system; hamster serine protease Casp, which degrades type I and IV collagen and fibronectin in the presence of calcium; mammalian complement-activating component of Ra-reactive factor (RARF), a protease that cleaves the C4 component of complement; vertebrate enteropeptidase (3.4.21.9), a type II membrane protein of the intestinal brush border, which activates trypsinogen; vertebrate bone morphogenic protein 1 (BMP-1), a protein which induces cartilage and bone formation and expressesmetalloendopeptidase activity; sea urchins blastula proteins BP10 and SpAN; Caenorhabditis elegans hypothetical proteins F42A10.8 and R151.5; neuropilin (A5 antigen), a calcium-independent cell adhesion molecule that functions during the formation of certain neuronal circuits; fibropellins I and III from sea urchin; mammalian hyaluronate-binding protein TSG-6 (or PS4), a serum and growth factor induced protein; mammalian spermadhesins; and Xenopusembryonic protein UVS.2, which is expressed during dorsoanterior development. (Interpro: IPR000859)

A sequence of about thirty to forty amino-acid residues long found in the sequence of epidermal growth factor (EGF) has been shown to be present, in a more or less conserved form, in a large number of other, mostly animal proteins. The list of proteins currently known to contain one or more copies of an EGF-like pattern is large and varied. The functional significance of EGF domains in what appear to be unrelated proteins is not yet clear. However, a common feature is that these repeats are found in the extracellular domain of membrane-bound proteins or in proteins known to be secreted (exception: prostaglandin G/H synthase). The EGF domain includes six cysteine residues which have been shown (in EGF) to be involved in disulfide bonds. The main structure is a two-stranded beta-sheet followed by a loop to a C-terminal short two-stranded sheet. Subdomains between the conserved cysteines vary in length. (Interpro:IPR000561)

The protein similarity information, expression pattern, and map location for the MEGF/FIBRILLIN-like protein and nucleic acid (NOV8) disclosed herein suggest that NOV8 protein may have important structural and/or physiological functions characteristic of the (epidermal growth factor) EGF family. Therefore, the NOV8 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV8 nucleic acids and proteins of the invention are useful in potential diagnostic and therapeutic applications implicated in various diseases and disorders described below and/or other pathologies. For example, the compositions of the present invention will have efficacy for treatment of patients suffering from diabetes, autoimmune disease, renal artery stenosis, interstitial nephritis, glomerulonephritis, polycystic kidney disease, systemic lupus erythematosus, renal tubular acidosis, IgA nephropathy, hypercalceimia and Lesch-Nyhan syndrome. NOV8 nucleic acid, or fragments thereof, may further be useful in diagnostic applications, wherein the presence or amount of the nucleic acid or the protein are to be assessed.

NOV8 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV8 protein has multiple hydrophilic regions, each of which can be used as an immunogen. This novel protein also has value in development of powerful assay system for functional analysis of various human disorders, which will help in understanding of pathology of the disease and development of new drug targets for various disorders.

NOV9

A disclosed NOV9 nucleic acid of 1425 nucleotides (also referred to as GMG55707_EXT.0.1da1) encoding a novel Growth/Differentiation Factor 6-like protein is shown in Table 9A. An open reading frame was identified beginning with an ATG initiation codon at nucleotides 31-33 and ending with a TAG codon at nucleotides 1396-1398. A putative untranslated region upstream from the initiation codon and downstream from the termination codon is underlined in Table 9A, and the start and stop codons are in bold letters.

TABLE 9A
NOV9 Nucleotide Sequence

| | |
|---|---|
| CTCCTGGGGAGACGCAGCCACTTGCCCGCCATGGATACTCCCAGGGTCCTGCTCTCGGCCGTCT | (SEQ ID NO:19) |
| TCCTCATCAGTTTTCTGTGGGATTTGCCCGGTTTCCAGCAGGCTTCCATCTCATCCTCCTGTTC | |
| GTCCGCCGAGCTGGGTTCCACCAAGGGCATGCGAAGCCGCAAGGAAGGCAAGATGCAGCGGGCG | |
| CCGCGCGACAGTGACGCGGGCCGGGAGGGCCAGGAACCACAGCCGCGGCCTCAGGACGAACCCC | |
| GGGCTCAGCAGCCCCGGGCGCAGGAGCCGCCAGGCAGGGGTCCGCGCGTGGTGCCCCACGACTA | |
| CATGCTGTCAATCTACAGGACTTACTCCATCGCTGAGAAGCTGGGCATCAATGCCAGCTTTTTC | |
| CAGTCTTCCAAGTCGGCTAATACGATCACCAGCTTTGTAGACAGGGGACTAGACGATCTCTCGC | |
| ACACTCCTCTCCGGAGACAGAAGTATTTGTTTGATGTGTCCATGCTCTCAGACAAAGAAGAGCT | |
| GGTGGGCGCGGAGCTGCGGCTCTTTCGCCAGGCGCCCTCAGCGCCCTGGGGGCCACCAGCCGGG | |
| CCGCTCCACGTGCAGCTCTTCCCTTGCCTTTCGCCCCTACTGCTGGACGCGCGGACCCTGGACC | |
| CGCAGGGGGCGCCGCCCGCCGGCTGGGAAGTCTTCGACGTGTGGCAGGGCCTGCGCCACCAGCC | |
| CTGGAAGCAGCTGTGCTTGGAGCTGCGGGCCGCATGGGGCGAGCTGGACGCCGGGGAGGCCGAG | |
| GCGCGCGCGCGGGGACCCCAGCAACCGCCGCCCCCGGACCTGCGGAGTCTGGGCTTCGGCCGGA | |
| GGGTGCGGCCTCCCCAGGAGCGGGCCCTGCTGGTGGTATTCACCAGATCCCAGCGCAAGAACCT | |
| GTTCGCAGAGATGCGCGAGCAGCTGGGCTCGGCCGAGGCTGCGGGCCCGGGCGCGGGCGCCGAG | |
| GGGTCGTCGCCGCCGCCGTCGGGCGCCCCGGATGCCAGGCCTTGGCTGCCCTCGCCCGGCCGCC | |
| GGCGGCGGCGCACGGCCTTCGCCAGTCGCCATGGCAAGCGGCACGGCAAGAAGTCCAGGCTACG | |
| CTGCAGCAAGAAGCCCCTGCACGTGAACTTCAAGGAGCTGGGCTGGGACGACTGGATTATCGCG | |
| CCCCTGGAGTACGAGGCCTATCACTGCGAGGGTGTATGCGACTTCCCGCTGCGCTCGCACCTGG | |
| AGCCCACCAACCACGCCATCATCCAGACGCTGATGAACTCCATGGACCCCGGCTCCACCCCGCC | |
| CAGCTGCTGCGTGCCCACCAAATTGACTCCCATCAGCATTCTATACATCGACGCGGGCAATAAT | |
| GTGGTCTACAAGCAGTACGAGGACATGGTGGTGGAGTCGTGCGGCTGCAGGTAGCGGTGCCTTT | |
| CCCGCCGCCTTGGCCCG | |

A disclosed NOV9 polypeptide (SEQ ID NO:20) encoded by SEQ ID NO:19 has 455 amino acid residues and is presented using the one-letter code in Table 9B. Signal P, Psort and/or Hydropathy results predict that NOV9 has a signal peptide and is likely to be localized extracellularly with a certainty of 0.5804. The most likely cleavage site for a NOV9 peptide is between amino acids 22 and 23, at LPG-FQ. NOV2 has a molecular weight of 50677 Daltons.

TABLE 9B
Encoded NOV9 protein sequence.

| | |
|---|---|
| MDTPRVLLSAVFLISFLWDLPGFQQASISSSCSSAELGSTKGMRSRKEGKMQRAPRDSDAGREGQ | (SEQ ID NO:20) |
| EPQPRPQDEPRAQQPRAQEPPGRGPRVVPHEYMLSIYRTYSIAEKLGINASFFQSSKSANTITSF | |
| VDRGLDDLSHTPLRRQKYLFDVSMLSDKEELVGAELRLFRQAPSAPWGPPAGPLHVQLFPCLSPL | |
| LLDARTLDPQGAPPAGWEVFDVWQGLRHQPWKQLCLELRAAWGELDAGEAEARARGPQQPPPPDL | |
| RSLGFGRRVRPPQERALLVVFTRSQRKNLFAEMREQLGSAEAAGPGAGAEGSWPPPSGAPDARPW | |
| LPSPGRRRRRTAFASRHGKRHGKKSRLRCSKKPLHVNFKELGWDDWIIAPLEYEAYHCEGVCDFP | |
| LRSHLEPTNHAIIQTLMNSMDPGSTPPSCCVPTKLTPISILYIDAGNNVVYKQYEDMVVESCGCR | |

The disclosed NOV9 amino acid sequence has 354 of 435 amino acid residues (81%) identical to, and 372 of 435 residues (85%) positive with, the Bos taurus 436 amino acid residue GROWTH/DIFFERENTIATION FACTOR 6 PRECURSOR (GDF-6) (CARTILAGE-DERIVED MORPHOGENETIC PROTEIN 2)(CDMP-2)ptnr:SWISSPROT-ACC:P55106)(E=6.3e⁻¹⁸⁵).

The NOV9 amino acid sequence also has 146 of 399 amino acid residues (36%) identical to, and 229 of 399 residues (57%) positive with, the 476 amino acid residue CGI-04 PROTEIN protein from Homo sapiens, (ptnr:SPTREMBL-ACC: Q9Y2Z4)(E=2.7e⁻⁶⁴).

NOV9 also has homology to the amino acid sequences shown in the BLASTP data listed in Table 9C.

TABLE 9C
BLAST results for NOV9

| Gene Index/ | | Length | Identity | Positives | |
|---|---|---|---|---|---|
| Identifier | Protein/Organism | (aa) | (%) | (%) | Expect |
| gi\|1707885\|sp\|P55106 | GROWTH/ | 436 | 335/438 | 350/438 | 1e−156 |
| \|GDF6_BOVIN | DIFFERENTIATION | | (76%) | (79%) | |
| | ATION FACTOR 6 | | | | |
| | PRECURSOR (GDF- | | | | |
| | 6) (CARTILAGE - | | | | |
| | DERIVED | | | | |
| | MORPHOGENETIC | | | | |
| | PROTEIN 2)(CDMP- | | | | |
| | 2) | | | | |
| gi\|5052013\|gb\| | growth and | 399 | 271/459 | 309/459 | 1e−125 |
| AAD38402.1\|AF155125_1 | differentiation | | (59%) | (67%) | |
| (AF155125) | factor 6 [Xenopus | | | | |
| | laevis] | | | | |
| gi\|914116\|gb\|AAB34226.1\| | TGF- | 154 | 235/381 | 274/381 | 1e−119 |
| | beta = transforming | | (61%) | (71%) | |
| | growth factor | | | | |
| | beta/Radar | | | | |
| | product [Danio | | | | |
| | rerio = zebrafish, | | | | |
| | embryos, Peptide, | | | | |
| | 354 aa] | | | | |
| gi\|1906321\|emb\| | Dynamo protein | 412 | 218/369 | 264/369 | 1e−109 |
| CAA68102.1\|(X99769) | [Danio rerio] | | (59%) | (71%) | |
| gi\|9802031\|gb\|AAF99597.1 | growth/differentiation | 413 | 198/385 | 250/385 | 2e−90 |
| \|AF239676 1 | factor 16 | | (51%) | (64%) | |
| (AF239676) | precursor protein | | | | |
| | [Xenopus laevis] | | | | |

The homology of these sequences is shown graphically in the ClustalW analysis shown in Table 9D.

[image]

[image]

[image]

Tables 9E-9G list the domain description from DOMAIN analysis results against NOV9. This indicates that the NOV9 sequence has properties similar to those of other proteins known to contain this domain.

TABLE 9E
Domain Analysis of NOV9

gnl|Smart|smart00204, TGFB, Transforming growth factor-beta (SEQ ID NO:77)
(TGF-beta) family; Family members are active as disulphide-linked
homo- or heterodimers. TGFB is a multifunctional peptide
that controls proliferation, differentiation, and other
functions in many cell types.
Length = 102 residues, 100.0% aligned
Score = 172 bits (437), Expect = 3e−44

| | | | |
|---|---|---|---|
| NOV9: | 354 | CSKKPLHVNFKELGWDDIIAPLEYEAYHCEGVCDFPLRSHLEPTNHAIIQTLMNSMDPG | 413 |
| | | \| + \|+\|+\|\|+\|\|\|\|\|\|\|\|\|\| \| \|\|+\|\|\| \| \|\|\| \| \|\|\|\|\|+\|+\|++++\|\|\| | |
| smart00204 | 1 | CRRHDLYVDFKDLGWDDWIIAPKGYNAYYCEGECPFPLSERLNATNHAIVQSLVHALDPG | 60 |
| NOV9: | 414 | STPPSCCVPTKLTPISILYIDAGNNVVYKQYEDMVVESCGCR | 455 |
| | | + \| \|\|\|\|\|\|\|+\|+\|+\|\| \| \|\|\| + \| +\|\|\|\| \|\|\|\| | |
| smart00204: | 61 | AVPKPCCVPTKLSPLSMLYYDDDGNVVLRNYPNMVVEECGCR | 102 |

TABLE 9F
Domain Analysis of NOV9

gnl|Pfam|pfam00019, TGF-beta, Transforming growth factor beta
like domain. (SEQ ID NO:78)
Length = 106 residues, 97.2% aligned
Score = 135 bits (340) Expect = 5e−33

| | | | |
|---|---|---|---|
| NOV9 | 354 | CSKKPLHVNFKELGWDDWIIAPLEYEAYHCEGVCDFPLRSHLEPT-NHAIIQTLMNSMDP | 412 |
| | | \| +\|+\|+\|++\|\|\|\|\|\|\|\|\| \|\|+\|\|\|\|\|\|\|+\| +\|\|\|\|+\|\|\|+ +\| | |
| pfam00019: | 4 | CRLRSLYVDFRDLGWGDWIIAPEGYIANYCSGSCPFPLRDYLNLSLNHAILQTLVRLRNP | 63 |
| NOV9: | 413 | GSTPPSCCVPTKLTPISILYIDAGNNVVYKQYEDMVVESCGCR | 455 |
| | | + \| \|\|\|\|\| \|+\|+\|+\|\|+\| +\|\|\| \| \| +\| \|\| \|\|\|\| | |
| pfam00019: | 64 | RAVPQPCCVPTALSPLSVLYLDDNSNVVLKVYPNMSVEECGCR | 106 |

TABLE 9G
Domain Analysis of NOV9

gnl|Pfam|pfam00688, TGFb_propeptide, TGF-beta propeptide. (SEQ ID NO:79)
Length = 226 residues, 82.7% aligned
Score = 87.0 bits (214), Expect = 2e−18

| | | | |
|---|---|---|---|
| NOV9: | 93 | VPHEYMLSIYRTYSIAEKLG------INASFFQSSKSANTITSFVDRGLDDLSHTP--LR | 144 |
| | | \|+\|\| +\| \| \|+ \|+ + + \|\|\|\| \|\| +\|\| + | |
| pfam00688: | 40 | SVPEFMLDLYNALSELEEGKVGRVPEISDYDGREAGRANTIRSFSHLEVDDFEESTPESH | 99 |
| NOV9: | 145 | RQKYLFDVSMLSDKEELVGAELRLFRQAPSAPWGPPAGPLHVQLFPCLSP-------LLL | 197 |
| | | \|+++ \|+\|\| + + \| \| \|\|\|\|\|+\| \|+++ \| \| \|\| | |
| pfam00688: | 100 | RKRFRFNVSSIPEGETLTAAELRLYRD---PLALRKRAEQRVEIYQLLKPGSDGSPTRLL | 156 |
| NOV9: | 198 | DARTLDPQGAPPAGWEVFDVWQGLRHQPWKQLCLELRAAWGELDAGEAEARARGPQQPPP | 257 |
| | | \|+\| +\| \|\| \|\|\| + \| \| \| + \| \| | |
| pfam00688: | 157 | DSRLVDASD--SGGWLSFDVTSAVNRWLSKPESNLGLQLEVECLCGHVDPRRAGLIG--- | 211 |
| NOV9: | 258 | PDLRSLGFGRRVRPPQERALLVVFT | 282 |
| | | \| \| + \|\|\| \| | |
| pfam00688: | 212 | ----------EPGPQQLQPLLVTFF | 226 |

Patp BLAST results for NOV9 include those listed in Table 9H.

TABLE 9H
Patp alignments of NOV9

| | | | Smallest |
|---|---|---|---|
| | | | Sum |
| Sequences producing High-scoring | Reading | High | Prob. |
| Segment Pairs: | Frame | Score | P (N) |
| patp:AAR95636 Cartilage-derived morph | +1 | 1795 | 3.3e−184 |
| protein-2 [Bos taurus], 436 aa | | | |
| patp:AAW26591 Human bone morph protein | +1 | 1747 | 4.0e−179 |
| BMP-13 [Homo sap], 321 aa | | | |

The above defined information for this invention suggests that this Growth/Differentiation Factor 6-like protein (NOV9) may function as a member of a “Growth/Differentiation Factor 6 family”. Therefore, the NOV9 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV9 nucleic acid encoding Growth/Differentiation Factor 6-like protein, and the Growth/Differentiation Factor 6-like protein of the invention, or fragments thereof, may further be useful in diagnostic applications, wherein the presence or amount of the nucleic acid or the protein are to be assessed. NOV9 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV9 protein has multiple hydrophilic regions, each of which can be used as an immunogen. In one embodiment, a contemplated NOV9 epitope is from about amino acids 25 to 95. In another embodiment, a NOV9 epitope is from about amino acids 120 to 140. In additional embodiments, NOV9 epitopes are from about amino acids 200 to 270 and from about amino acids 275 to 360. These novel proteins can be used in assay systems for functional analysis of various human disorders, which are useful in understanding of pathology of the disease and development of new drug targets for various disorders.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17 and 19, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17 and 19 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:1, 3, 5, 7, 9, 11, 13, 15, 17, and 19, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17 and 19, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 5, 7, 9, 11, 13, 15, 17 or 19 is one that is sufficiently complementary to the nucleotide sequence shown SEQ ID NOS:1, 3, 5, 7, 9, 11, 13, 15, 17 or 19 that it can hydrogen bond with little or no mismatches to the nucleotide sequence shown SEQ ID NOS:1, 3, 5, 7, 9, 11, 13, 15, 17 and 19, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 17 and 19, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 17 or 19; or an anti-sense strand nucleotide sequence of SEQ ID NOS:1, 3, 5, 7, 9, 11, 13, 15, 17 or 19; or of a naturally occurring mutant of SEQ ID NOS:1, 3, 5, 7, 9, 11, 13, 15, 17 and 19.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17 or 19, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 17 and 19 due to degeneracy of the genetic code and thus encode the same NOVX proteins as that encoded by the nucleotide sequences shown in SEQ ID NOS:1, 3, 5, 7, 9, 11, 13, 15, 17 and 19. In another embodiment, an isolated nucleic acid molecule of the invention has a nucleotide sequence encoding a protein having an amino acid sequence shown in SEQ ID NOS:2, 4, 6, 8, 10, 12, 14, 16, 18 or 20.

In addition to the human NOVX nucleotide sequences shown in SEQ ID NOS:1, 3, 5, 7, 9, 11, 13, 15, 17 and 19, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 5, 7, 9, 11, 13, 15, 17 and 19 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17 and 19. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at

pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:1, 3, 5, 7, 9, 11, 13, 15, 17 and 19, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17 and 19, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS:1, 3, 5, 7, 9, 11, 13, 15, 17 and 19, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 17 and 19, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18 or 20. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:1, 3, 5, 7, 9, 11, 13, 15, 17 and 19 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18 and 20. Preferably, the protein encoded by the nucleic acid molecule is at least about 60% homologous to SEQ ID NOS:2, 4, 6, 8, 10, 12, 14, 16, 18 and 20; more preferably at least about 706 homologous SEQ ID NOS:2, 4, 6, 8, 10, 12, 14, 16, 18 or 20; still more preferably at least about 80% homologous to SEQ ID NOS:2, 4, 6, 8, 10, 12, 14, 16, 18 or 20; even more preferably at least about 90% homologous to SEQ ID NOS:2, 4, 6, 8, 10, 12, 14, 16, 18 or 20; and most preferably at least about 95% homologous to SEQ ID NOS:2, 4, 6, 8, 10, 12, 14, 16, 18 or 20.

An isolated nucleic acid molecule encoding an NOVX protein homologous to the protein of SEQ ID NOS:2, 4, 6, 8, 10, 12, 14, 16, 18 or 20 can be created by introducing one or more nucleotide substitutions, additions or deletions into the nucleotide sequence of SEQ ID NOS:1, 3, 5, 7, 9, 11, 13, 15, 17 and 19, such that one or more amino acid substitutions, additions or deletions are introduced into the encoded protein.

Mutations can be introduced into SEQ ID NOS:1, 3, 5, 7, 9, 11, 13, 15, 17 and 19 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:1, 3, 5, 7, 9, 11, 13, 15, 17 and 19, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof, (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17 and 19, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18 or 20, or antisense nucleic acids complementary to an NOVX nucleic acid sequence of SEQ ID NOS:1, 3, 5, 7, 9, 11, 13, 15, 17 and 19, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisensc nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 5, 7, 9, 11, 13, 15, 17 and 19). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g. the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. BioorgMed Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as anfisense or antigene agents for sequence-specific modulation of gene expression by, eg., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art For example, PNA-DNA chimeras of NOVX can be generated that may combine the advantageous properties of PNA and DNA. Such chimeras allow DNA recognition enzymes (e.g, RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′PNA segment and a 3′DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharnz. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18 or 20. The invention also includes a mutant or variant protein any of whose residues may be changed from the corresponding residues shown in SEQ ID NOS:2, 4, 6, 8, 10, 12, 14, 16, 18 or 20 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18 or 20) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18 or 20. In other embodiments, the NOVX protein is substantially homologous to SEQ ID NOS:2, 4, 6, 8, 10, 12, 14, 16, 18 or 20, and retains the functional activity of the protein of SEQ ID NOS:2, 4, 6, 8, 10, 12, 14, 16, 18 or 20, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18 or 20, and retains the functional activity of the NOVX proteins of SEQ ID NOS:2, 4, 6, 8, 10, 12, 14, 16, 18 or 20.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17 and 19.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g. A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18 or 20), whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences arc ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S1 nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an Fab expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragmnents, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Gucrin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can bc subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al, Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “filly human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatrnent of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (JNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fe region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al., Cancer Research, 53: 2560-2565 (1993). Alternatively, an antibody can be engineered that has dual Fe regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g. for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerytlirin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most conunonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g. NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 9337943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g. the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilglunan, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 17 and 19 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and arc described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 17 and 19), but more preferably, is a non-human homologue of a human NOVX gene. For example, a mouse homologue of human NOVX gene of SEQ ID NOS:1, 3, 5, 7, 9, 11, 13, 15, 17 and 19 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g. a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring home of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for case of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. USA. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylanuminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-inked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 17 and 19, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids arc prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like coleemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17 and 19 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoictic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 17 and 19, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g. Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g. Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g. Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chein. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g. Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g. in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e g, Eichelbaum, 1996. Clin. Exp. Pharmacol. Physiol, 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof, (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (eg., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLE 1

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources), Panel 4 (containing cells and cell lines from normal cells and cells related to inflammatory conditions) and Panel CNSD.01 (containing samples from normal and diseased brains).

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

In the results for Panel 1, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural efflusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from humnan malignancies and in cases where indicated many malignant tissues have “matched margins” obtained from noncancerous tissue iust adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN garmma at approximately 20-50 ng/ml, IL4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Iiyclone), 100 μM non essential amino acids (Gibco/Life Technologies, Rockville, Md.), 1 mM sodium pyruvate (Gibco), mercaptoethanol 5.5×10⁵ M (Gibco), and 10 mM Hepes (Gibco) and Interleukin 2 for 46 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM HIepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD9 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μl/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 45 days, the Th1, Th2 and Tr1 lymphocytes were washed and then expanded again with IL-2 for 47 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 107 cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supemuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

NOV1: Asparaginyl Endopeptidase-Like

Expression of gene NOV1 (GMba261alA) was assessed using the primer-probe set Ag2462, described in Table B.

TABLE B
Probe Name Ag2462

| | | | | Start | SEQ ID |
|---|---|---|---|---|---|
| Primers | Sequences | TM | Length | Position | NO: |
| Forward | 5′-ATCATGCAGTACGGAAACGA-3′ | 59.2 | 20 | 864 | 80 |
| Probe | TET-5′-CGATCTCCACATTAAAAGTGATGCAG-3′-TAMRA | 65 | 26 | 886 | 81 |
| Reverse | 5′-GGAGAACTGGCTTTGTGTTTC-3′ | 58.8 | 21 | 923 | 82 |

The NOV1 gene is expressed at low/undetectable levels across the samples on Panels 1.3D and 4D (data not shown). Results from an experiment examining NOV1 gene expression on Panel 2D could not be evaluated due to chemistry problems (data not shown).

NOV3: Melastatin-Like

Expression of gene NOV3 (32073570 EXT) was assessed using the primer-probe sets Ag534, Ag535, and Ag3690, described in Tables B, C and D. Results of the RTQ-PCR runs are shown in Tables F, G, and H.

TABLE C
Probe Name Ag534

| | | | | Start | SEQ ID |
|---|---|---|---|---|---|
| Primers | Sequences | TM | Length | Position | NO: |
| Forward | 5′-TCAGTGATACAATTGCCATAATTTCTTT-3′ | | 28 | 68 | 83 |
| Probe | FAM-5′-TTTGCTCCAAATCTTAGTCCAAATCCAATGAA-3′-TAMRA | | 32 | 97 | 84 |
| Reverse | 5′-AAAAACATGATTATCATATGCATTTGC-3′ | | 27 | 139 | 85 |

TABLE D
Probe Name Ag535

| | | | | Start | SEQ ID |
|---|---|---|---|---|---|
| Primers | Sequences | TM | Length | Position | NO: |
| Forward | 5′-TTTACAAGTGAAGGCAATTTCCAA-3′ | | 24 | 108 | 86 |
| Probe | TET-5′-AGCCATAATAAAATGATAACGCTGGTACTTCCATACAAT-3′-TAMRA | | 39 | 133 | 87 |
| Reverse | 5′-GAGGCAGAACTGGTTTCTCATGA-3′ | | 23 | 174 | 88 |

TABLE E
Probe Name Ag3690

| | | | | Start | SEQ ID |
|---|---|---|---|---|---|
| Primers | Sequences | TM | Length | Position | NO: |
| Forward | 5′-AGACGTCAAACAGGGAAATCTT-3′ | 59.2 | 22 | 1720 | 89 |
| Probe | FAM-5′-CCTCCAGGATATAAGATCACTCTGATTGA-3′-TAMRA | 64.8 | 29 | 1742 | 90 |
| Reverse | 5′-TCTGTAGGTTCCTCCCATGAG-3′ | 59.2 | 21 | 1793 | 91 |

TABLE F
Panel 1.1

| | Relative | | Relative |
|---|---|---|---|
| | Expression(%) | | Expression(%) |
| | 1..1tm836f_{—} | | 1..1tm836f_{—} |
| Tissue Name | ag534 | Tissue Name | ag534 |
| Adipose | 3.8 | Renal ca. TK-10 | 25.9 |
| Adrenal gland | 21.2 | Renal ca. UO-31 | 6.2 |
| Bladder | 57.8 | Renal ca. RXF 393 | 4.4 |
| Brain (amygdala) | 3.7 | Liver | 59.5 |
| Brain (cerebellum) | 59.9 | Liver (fetal) | 13.8 |
| Brain (hippocampus) | 15.0 | Liver ca. (hepatoblast) HepG2 | 13.7 |
| Brain (substantia nigra) | 21.5 | Lung | 5.6 |
| Brain (thalamus) | 7.6 | Lung (fetal) | 6.6 |
| Cerebral Cortex | 12.9 | Lung ca (non-s.cell) HOP-62 | 50.3 |
| Brain (fetal) | 17.9 | Lung ca. (large cell) NCI-H460 | 47.6 |
| Brain (whole) | 9.9 | Lung ca. (non-s.cell) NCI-H23 | 27.7 |
| CNS ca. (glio/astro) U-118-MG | 12.3 | Lung ca. (non-s.cl) NCI-H522 | 74.2 |
| CNS ca. (astro) SF-539 | 27.0 | Lung ca. (non-sm. cell) A549 | 32.1 |
| CNS ca. (astro) SNB-75 | 7.6 | Lung ca. (s.cell var.) SHP-77 | 7.6 |
| CNS ca. (astro) SW1783 | 4.9 | Lung ca. (small Cell) LX-1 | 52.5 |
| CNS ca. (glio) U251 | 18.0 | Lung ca. (small cell) NCI-H69 | 39.2 |
| CNS ca. (glio) SF-295 | 31.2 | Lung ca. (squam.) SW 900 | 12.6 |
| CNS ca. (glio) SNB-19 | 35.6 | Lung ca. (squam.) NCI-H596 | 34.6 |
| CNS ca. (glio/astro) U87-MG | 35.4 | Lymph node | 10.4 |
| CNS ca.* (neuro; met) SK-N- | 32.5 | Spleen | 6.4 |
| AS | | | |
| Mammary gland | 10.7 | Thymus | 13.1 |
| Breast ca. BT-549 | 8.0 | Ovary | 2.9 |
| Breast ca. MDA-N | 32.1 | Ovarian ca. IGROV-1 | 21.9 |
| Breast ca.* (pl. effusion) T47D | 62.8 | Ovarian ca. OVCAR-3 | 11.7 |
| Breast ca.* (pl. effusion) MCF-7 | 98.6 | Ovarian ca. OVCAR-4 | 4.5 |
| Breast ca.* (pl.ef) MDA-MB- | 7.4 | Ovarian ca. OVCAR-5 | 42.3 |
| 231 | | | |
| Small intestine | 24.8 | Ovarian ca. OVCAR-8 | 51.4 |
| Colorectal | 3.1 | Ovarian ca.* (ascites) SK-OV-3 | 28.1 |
| Colon ca. HT29 | 24.7 | Pancreas | 39.5 |
| Colon ca. CaCo-2 | 48.0 | Pancreatic ca. CAPAN 2 | 4.3 |
| Colon ca. HCT-15 | 15.3 | Pituitary gland | 28.7 |
| Colon ca. HCT-116 | 21.3 | Placenta | 27.5 |
| Colon ca. HCC-2998 | 53.2 | Prostate | 23.0 |
| Colon ca. SW480 | 5.2 | Prostate ca.* (bone met) PC-3 | 32.1 |
| Colon ca.* (SW480 met) SW620 | 44.4 | Salivary gland | 41.2 |
| Stomach | 13.0 | Trachea | 15.3 |
| Gastric ca.* (liver met) NCI- | 67.4 | Spinal cord | 14.1 |
| N87 | | | |
| Heart | 44.1 | Testis | 9.8 |
| Fetal Skeletal | 6.1 | Thyroid | 19.2 |
| Skeletal muscle | 54.0 | Uterus | 17.1 |
| Endothelial cells | 17.2 | Melanoma M14 | 26.6 |
| Heart (fetal) | 6.5 | Melanoma LOX IMVI | 5.3 |
| Kidney | 100.0 | Melanoma UACC-62 | 15.5 |
| Kidney (fetal) | 30.4 | Melanoma SK-MEL-28 | 80.7 |
| Renal ca. 786-0 | 15.9 | Melanoma* (met) SK-MEL-5 | 39.5 |
| Renal ca. A498 | 16.4 | Melanoma Hs688(A).T | 14.9 |
| Renal ca. ACHN | 11.3 | Melanoma* (met) Hs688(B).T | 16.3 |

TABLE G
Panel 1.2

| | Relative | | Relative |
|---|---|---|---|
| | Expression(%) | | Expression(%) |
| | 1.2tm874t_{—} | | 1.2tm874t_{—} |
| Tissue Name | ag535 | Tissue Name | ag535 |
| Endothelial cells | 10.2 | Renal ca. 786-0 | 14.6 |
| Heart (fetal) | 1.5 | Renal ca. A498 | 21.8 |
| Pancreas | 94.0 | Renal ca. RXF 393 | 5.5 |
| Pancreatic ca. CAPAN 2 | 8.4 | Renal ca. ACHN | 13.4 |
| Adrenal Gland (new lot*) | 41.5 | Renal ca. UO-31 | 5.8 |
| Thyroid | 29.1 | Renal ca. TK-10 | 22.2 |
| Salivary gland | 47.0 | Liver | 56.3 |
| Pituitary gland | 48.3 | Liver fetal | 20.6 |
| Brain (fetal) | 17.1 | Liver ca. (hepatoblast) HepG2 | 11.3 |
| Brain (whole) | 41.8 | Lung | 14.0 |
| Brain (amygdala) | 9.3 | Lung (fetal) | 20.7 |
| Brain (cerebellum) | 20.0 | Lung ca. (small cell) LX-1 | 48.3 |
| Brain (hippocampus) | 20.7 | Lung ca. (small cell) NCI-H69 | 27.4 |
| Brain (thalamus) | 17.6 | Lung ca. (s.cell var.) SHP-77 | 5.6 |
| Cerebral Cortex | 11.9 | Lung ca. (large cell) NCI-H460 | 61.1 |
| Spinal cord | 12.0 | Lung ca. (non-sm. cell) A549 | 25.9 |
| CNS ca. (glio/astro) U87-MG | 45.1 | Lung ca. (non-s.cell) NCI-H23 | 22.4 |
| CNS ca. (glio/astro) U-118-MG | 20.0 | Lung ca (non-s.cell) HOP-62 | 34.9 |
| CNS ca. (astro) SW1783 | 6.2 | Lung ca. (non-s.cl) NCI-H522 | 100.0 |
| CNS ca.* (neuro; met) SK-N- | 34.4 | Lung ca. (squam.) SW 900 | 15.8 |
| AS | | | |
| CNS ca. (astro) SF-539 | 22.2 | Lung ca. (squam.) NCI-H596 | 40.3 |
| CNS ca. (astro) SNB-75 | 9.6 | Mammary gland | 30.4 |
| CNS ca. (glio) SNB-19 | 31.0 | Breast ca.* (pl. effusion) MCF-7 | 70.7 |
| CNS ca. (glio) U251 | 24.7 | Breast ca.* (pl.ef) MDA-MB- | 6.6 |
| | | 231 | |
| CNS ca. (glio) SF-295 | 27.7 | Breast ca.* (pl. effusion) T47D | 37.9 |
| Heart | 49.3 | Breast ca. BT-549 | 16.6 |
| Skeletal Muscle (new lot*) | 48.6 | Breast ca. MDA-N | 31.4 |
| Bone marrow | 23.2 | Ovary | 2.5 |
| Thymus | 17.7 | Ovarian ca. OVCAR-3 | 25.5 |
| Spleen | 22.5 | Ovarian ca. OVCAR-4 | 5.6 |
| Lymph node | 33.0 | Ovarian ca. OVCAR-5 | 45.1 |
| Colorectal | 1.5 | Ovarian ca. OVCAR-8 | 23.3 |
| Stomach | 24.7 | Ovarian ca. IGROV-1 | 27.2 |
| Small intestine | 34.4 | Ovarian ca.* (ascites) SK-OV-3 | 33.9 |
| Colon ca. SW480 | 5.0 | Uterus | 21.2 |
| Colon ca.* (SW480 | 25.3 | Placenta | 60.7 |
| met) SW620 | | | |
| Colon ca. HT29 | 20.3 | Prostate | 25.3 |
| Colon ca. HCT-116 | 20.4 | Prostate ca.* (bone met) PC-3 | 59.9 |
| Colon ca. CaCo-2 | 39.8 | Testis | 37.1 |
| 8329 CC Well to Mod Diff | 2.5 | Melanoma Hs688(A).T | 14.5 |
| (ODO3866) | | | |
| Colon ca. HCC-2998 | 54.7 | Melanoma* (met) Hs688(B).T | 18.7 |
| Gastric ca.* (liver met) NCI- | 69.3 | Melanoma UACC-62 | 19.1 |
| N87 | | | |
| Bladder | 43.8 | Melanoma M14 | 25.2 |
| Trachea | 13.2 | Melanoma LOX IMVI | 4.2 |
| Kidney | 48.3 | Melanoma* (met) SK-MEL-5 | 50.0 |
| Kidney (fetal) | 51.8 | Adipose | 5.1 |

TABLE H
Panel 4.1D

| | Relative | | Relative |
|---|---|---|---|
| | Expression (%) | | Expression (%) |
| | 4.1dx4tm5979- | | 4.1dx4tm5979- |
| Tissue Name | f_3690_b2 | Tissue Name | f_3690_b2 |
| 93768_Secondary Th1_anti- | 70.2 | 93100_HUVEC | 22.8 |
| CD28/anti-CD3 | | (Endothelial)_IL-1b | |
| 93769_Secondary Th2_anti- | 77.8 | 93799_HUVEC | 33.9 |
| CD28/anti-CD3 | | (Endothelial)_IFN gamma | |
| 93770_Secondary Tr1_anti- | 79.9 | 93102_HUVEC | 25.6 |
| CD28/anti-CD3 | | (Endothelial)_TNF alpha + IFN | |
| | | gamma | |
| 93573_Secondary Th1_resting | 28.9 | 93101_HUVEC | 29.5 |
| day 4-6 in IL-2 | | (Endothelial)_TNF alpha + IL4 | |
| 93572_Secondary Th2_resting | 39.2 | 93781_HUVEC | 14.8 |
| day 4-6 in IL-2 | | (Endothelial)_IL-11 | |
| 93571_Secondary Tr1_resting | 38.9 | 93583_Lung Microvascular | 45.4 |
| day 4-6 in IL-2 | | Endothelial Cells_none | |
| 93568_primary Th1_anti- | 64.2 | 93584_Lung Microvascular | 44.1 |
| CD28/anti-CD3 | | Endothelial Cells_TNFa (4 ng/ml) | |
| | | and IL1b (1 ng/ml) | |
| 93569_primary Th2_anti- | 79.9 | 92662_Microvascular Dermal | 35.4 |
| CD28/anti-CD3 | | endothelium_none | |
| 93570_primary Tr1_anti- | 76.7 | 92663_Microsvasular Dermal | 34.8 |
| CD28/anti-CD3 | | endothelium_TNFa (4 ng/ml) | |
| | | and IL1b (1 ng/ml) | |
| 93565_primary Th1_resting dy | 45.1 | 93773_Bronchial | 57.5 |
| 4-6 in IL-2 | | epithelium_TNFa (4 ng/ml) and | |
| | | IL1b (1 ng/ml)** | |
| 93566_primary Th2_resting dy | 38.2 | 93347_Small Airway | 12.4 |
| 4-6 in IL-2 | | Epithelium_none | |
| 93567_primary Tr1_resting dy | 64.7 | 93348_Small Airway | 23.2 |
| 4-6 in IL-2 | | Epithelium_TNFa (4 ng/ml) | |
| | | and IL1b (1 ng/ml) | |
| 93351_CD45RA CD4 | 52.9 | 92668_Coronery Artery | 26.9 |
| lymphocyte_anti-CD28/anti- | | SMC_resting | |
| CD3 | | | |
| 93352_CD45RO CD4 | 84.3 | 92669_Coronery Artery | 20.4 |
| lymphocyte_anti-CD28/anti- | | SMC_TNFa (4 ng/ml) and IL1b | |
| CD3 | | (1 ng/ml) | |
| 93251_CD8 Lymphocytes_anti- | 75.9 | 93107_astrocytes_resting | 20.5 |
| CD28/anti-CD3 | | | |
| 93353_chronic CD8 | 74.5 | 93108_astrocytes_TNFa (4 | 22.1 ng/ml) |
| Lymphocytes 2ry_resting dy 4-6 | | and IL1b (1 ng/ml) | |
| in IL-2 | | | |
| 93574_chronic CD8 | 33.1 | 92666_KU-812 | 45.2 |
| Lymphocytes 2ry_activated | | (Basophil)_resting | |
| CD3/CD28 | | | |
| 93354_CD4_none | 26.8 | 92667_KU-812 | 85.8 |
| | | (Basophil)_PMA/ionoycin | |
| 93252_Secondary | 49.8 | 93579_CCD1106 | 40.0 |
| Th1/Th2/Tr1_anti-CD95 CH11 | | (Keratinocytes)_none | |
| 93103_LAK cells_resting | 57.9 | 93580_CCD1106 | 33.1 |
| | | (Keratinocytes)_TNFa and | |
| | | IFNg** | |
| 93788_LAK cells_IL-2 | 66.3 | 93791_Liver Cirrhosis | 18.2 |
| 93787_LAK cells_IL-2 + IL-12 | 52.0 | 93577_NCI-H292 | 31.9 |
| 93789_LAK cells_IL-2 + IFN | 68.6 | 93358_NCI-H292_IL-4 | 38.3 |
| gamma | | | |
| 93790_LAK cells_IL-2 + IL-18 | 68.3 | 93360_NCI-H292_IL-9 | 64.7 |
| 93104_LAK | 56.7 | 93359_NCI-H292_IL-13 | 38.0 |
| cells_PMA/ionomycin and IL-18 | | | |
| 93578_NK Cells IL-2_resting | 66.8 | 93357_NCI-H292_IFN gamma | 50.0 |
| 93109_Mixed Lymphocyte | 80.6 | 93777_HPAEC_- | 27.9 |
| Reaction_Two Way MLR | | | |
| 93110_Mixed Lymphocyte | 59.6 | 93778_HPAEC_IL-1 beta/TNA | 49.2 |
| Reaction_Two Way MLR | | alpha | |
| 93111_Mixed Lymphocyte | 44.1 | 93254_Normal Human Lung | 51.0 |
| Reaction_Two Way MLR | | Fibroblast_none | |
| 93112_Mononuclear Cells | 22.5 | 93253_Normal Human Lung | 18.4 |
| (PBMCs)_resting | | Fibroblast_TNFa (4 ng/ml) and | |
| | | IL-1b (1 ng/ml) | |
| 93113_Mononuclear Cells | 74.7 | 93257_Normal Human Lung | 38.8 |
| (PBMCs)_PWM | | Fibroblast_IL-4 | |
| 93114_Mononuclear Cells | 60.2 | 93256_Normal Human Lung | 52.7 |
| (PBMCs)_PHA-L | | Fibroblast_IL-9 | |
| 93249_Ramos (B cell)_none | 100.0 | 93255_Normal Human Lung | 38.1 |
| | | Fibroblast_IL-13 | |
| 93250_Ramos (B | 89.2 | 93258_Normal Human Lung | 41.9 |
| cell)_ionomycin | | Fibroblast_IFN gamma | |
| 93349_B lymphocytes_PWM | 50.0 | 93106_Dermal Fibroblasts | 52.9 |
| | | CCD1070_resting | |
| 93350_B lymphoytes_CD40L | 88.8 | 93361_Dermal Fibroblasts | 84.9 |
| and IL-4 | | CCD1070_TNF alpha 4 ng/ml | |
| 92665_EOL-1 | 54.4 | 93105_Dermal Fibroblasts | 27.9 |
| (Eosinophil)_dbcAMP | | CCD1070_IL-1 beta 1 ng/ml | |
| differentiated | | | |
| 93248_EOL-1 | 73.4 | 93772_dermal fibroblast_IFN | 30.1 |
| (Eosinophil)_dbcAMP/PMAionomycin | | gamma | |
| 93356_Dendritic Cells_none | 60.0 | 93771_dermal fibroblast_IL-4 | 80.5 |
| 93355_Dendritic Cells_LPS | 53.3 | 93892_Dermal fibroblasts_none | 37.8 |
| 100 ng/ml | | | |
| 93775_Dendritic Cells_anti- | 61.3 | 99202_Neutrophils_TNFa + LPS | 29.0 |
| CD40 | | | |
| 93774_Monocytes_resting | 60.1 | 99203_Neutrophils_none | 13.9 |
| 93776_Monocytes_LPS 50 | 55.5 | 735010_Colon_normal | 21.7 |
| ng/ml | | | |
| 93581_Macrophages_resting | 49.1 | 735019_Lung_none | 30.3 |
| 93582_Macrophages_LPS 100 | 29.1 | 64028-1_Thymus_none | 92.2 |
| ng/ml | | | |
| 93098_HUVEC | 16.2 | 64030-1_Kidney_none | 75.0 |
| (Endothelial)_none | | | |
| 93099_HUVEC | 27.9 | | |
| (Endothelial)_starved | | | |

Panel 1.1 Summary: Ag534 The NOV3 gene encodes a protein with homology to melastatin, a member of the transient receptor potential (Trp) family of calcium ion channels. The NOV3 gene is expressed at moderate to high levels across all the samples on this panel with the highest expression detected in kidney (CT=25). Interestingly, defects in ion channels are associated with kidney disorders, such as Bartter's syndrome, policystic kidney disease and Dent's disease (Dworakowska and Dolowy, Ion channels-related diseases. Acta Biochim Pol 47: 685-703, 2000), suggesting that the NOV3 gene may also play a role in kidney homeostasis.

Furthermore, this gene is expressed in a variety of metabolically relevant tissues, including adrenal gland, heart, skeletal muscle, liver, pancreas, pituitary gland, and thyroid. Therefore, as a classical drug target, the NOV3 protein may be useful for the treatment of disease in any or all of these tissues, including diabetes and obesity. In support of this hypothesis, mutations in ion channels have previously been associated with hyperinsulinemic hypoglycemia of infancy (Dworakowska and Dolowy, Ion channels-related diseases. Acta Biochim Pol 47: 685-703, 2000).

Among CNS samples, the NOV3 gene is expressed in hippocampus, substantia nigra, thalamus, and cerebral cortex with highest expression detected in the cerebellum (CT values<30). The protein encoded by the NOV3 gene shows considerable homology to known ion channels, which are the primary targets of all known antiepileptics. Furthermore, all gene mutations known to cause epilepsy or seizure disorders are found in ion channels (Dworakowska and Dolowy, Ion channels-related diseases. Acta Biochim Pol 47: 685-703, 2000; Li and Lester, Ion channel diseases of the central nervous system. CNS Drug Rev 7: 214-240, 2001). Two established antiepileptics (valproate and carbamazepine) also have efficacy in the treatment of bipolar disorder. Therefore, therapeutic modulation of this gene or its protein product may be beneficial in the treatment of these disorders.

Interestingly, it also appears that there is a difference in NOV3 gene expression between several adult tissues and their fetal counterparts. Specifically, expression of this gene is significantly higher in adult kidney, liver, skeletal muscle and heart when compared to the corresponding fetal tissues. Thus, the expression of the NOV3 gene could be used as a marker of adult tissues, or alternatively its relative absence could be used as a marker of fetal tissues. Since fetal tissues show potential use for organ regeneration, the expression of this gene may be inhibitory to organogenesis. Thus, the therapeutic modulation of the activity of the NOV3 gene product, through the use of small molecule drugs or antibodies, might be of use for the treatment of diseases whose pathology is characterized by organ degeneration.

Panel 1.2 Summary: Ag535 The NOV3 gene is expressed at moderate to high levels across all the samples on this panel with the highest expression detected in lung cancer cell line NCI-H522 (CT=25). In general, the pattern of NOV3 gene expression is consistent with that observed in Panel 1.1; see Panel 1.1 for discussion of expression pattern in CNS and metabolically relevant tissues.

Interestingly, there appears to be a difference in NOV3 gene expression between fetal and adult liver. In addition, this gene is also highly expressed in pancreas (CT=25). Thus, the relative expression of the NOV3 gene might be useful as a marker of pancreas tissue. Furthermore, since this gene appears to be differentially expressed in adult and fetal liver, and that fetal liver represents a state of organogenesis, the therapeutic down-modulation of this gene product, through the use of small molecule drugs or antibodies might be of use in the treatment of diseases involving liver degeneration.

Panel 4.1D Summary: Ag3690 The NOV3 gene is expressed at low to moderate levels in each of the cells and tissues examined on this panel. This observation suggests that this gene plays an important role in a variety of immunologically relevant cell types. Interestingly, calcium release activated calcium channels have been shown to be required for T cell activation, cytokine synthesis, and proliferation (Lepple-Wienhues et al., Stimulation of CD95 (Fas) blocks T lymphocyte calcium channels through sphingomyelinase and sphingolipids. Proc Natl Acad. Sci. USA 96: 13795-13800, 1999).

NOV4: Leucine-Rich Repeat-Like

Expression of gene NOV4 (124141642_EXT) was assessed using the primer-probe sets Ag1388 and Ag2455, described in Tables I and J. Results of the RTQ-PCR runs are shown in Tables K, L, M, N and O.

TABLE I
Probe Name Ag1388

| | | | | Start | SEQ ID |
|---|---|---|---|---|---|
| Primers | Sequences | TM | Length | Position | NO: |
| Forward | 5′-CTGGTAATCCTGCTGGACTACA-3′ | 59.3 | 22 | 412 | 92 |
| Probe | FAM-5′-CTTTCCAGGACCTGCACAGCCTG-3′-TAMRA | 69.5 | 23 | 434 | 93 |
| Reverse | 5′-AGACGAATACCAGGTCGTTGT-3′ | 58.6 | 21 | 476 | 94 |

TABLE J
Probe Name Ag2455

| | | | | Start | SEQ ID |
|---|---|---|---|---|---|
| Primers | Sequences | TM | Length | Position | NO: |
| Forward | 5′-GCTGGTAATCCTGCTGGACTA-3′ | 59.3 | 21 | 475 | 95 |
| Probe | FAM-5′-ACTTTCCAGGACCTGCACAGCCTG-3′-TAMRA | 69.9 | 24 | 497 | 96 |
| Reverse | 5′-AGACGAATACCAGGTCGTTGT-3′ | 58.6 | 21 | 540 | 97 |

TABLE K
Panel 1.2

| | Relative | | Relative |
|---|---|---|---|
| | Expression (%) | | Expression (%) |
| | 1.2tm1617f_{—} | | 1.2tm1617f_{—} |
| Tissue Name | ag1388 | Tissue Name | ag1388 |
| Endothelial cells | 0.9 | Renal ca. 786-0 | 0.3 |
| Heart (fetal) | 0.7 | Renal ca. A498 | 0.2 |
| Pancreas | 0.2 | Renal ca. RXF 393 | 1.7 |
| Pancreatic ca. CAPAN 2 | 0.2 | Renal ca. ACHN | 0.0 |
| Adrenal Gland (new lot*) | 6.1 | Renal ca. UO-31 | 0.2 |
| Thyroid | 0.9 | Renal ca. TK-10 | 0.0 |
| Salivary gland | 18.3 | Liver | 1.7 |
| Pituitary gland | 0.0 | Liver (fetal) | 2.0 |
| Brain (fetal) | 1.1 | Liver ca. (hepatoblast) HepG2 | 2.9 |
| Brain (whole) | 10.5 | Lung | 1.6 |
| Brain (amygdala) | 7.4 | Lung (fetal) | 0.4 |
| Brain (cerebellum) | 100.0 | Lung ca. (small cell) LX-1 | 1.3 |
| Brain (hippocampus) | 12.4 | Lung ca. (small cell) NCI-H69 | 6.6 |
| Brain (thalamus) | 20.2 | Lung ca. (s.cell var.) SHP-77 | 0.0 |
| Cerebral Cortex | 20.4 | Lung ca. (large cell) NCI-H460 | 0.9 |
| Spinal cord | 1.6 | Lung ca. (non-sm. cell) A549 | 2.7 |
| CNS ca. (glio/astro) U87-MG | 3.3 | Lung ca. (non-s.cell) NCI-H23 | 0.3 |
| CNS ca. (glio/astro) U-118-MG | 3.4 | Lung ca (non-s.cell) HOP-62 | 0.0 |
| CNS ca. (astro) SW1783 | 1.1 | Lung ca. (non-s.cl) NCI-H522 | 0.5 |
| CNS ca.* (neuro; met) SK-N- | 2.5 | Lung ca. (squam.) SW 900 | 1.3 |
| AS | | | |
| CNS ca. (astro) SF-539 | 1.3 | Lung ca. (squam.) NCI-H596 | 3.3 |
| CNS ca. (astro) SNB-75 | 1.7 | Mammary gland | 3.3 |
| CNS ca. (glio) SNB-19 | 0.7 | Breast ca.* (pl. effusion) MCF-7 | 6.3 |
| CNS ca. (glio) U251 | 0.0 | Breast ca.* (pl.ef) MDA-MB- | 0.2 |
| | | 231 | |
| CNS ca. (glio) SF-295 | 0.2 | Breast ca.* (pl. effusion) T47D | 15.6 |
| Heart | 1.0 | Breast ca. BT-549 | 0.5 |
| Skeletal Muscle (new lot*) | 0.2 | Breast ca. MDA-N | 0.3 |
| Bone marrow | 38.4 | Ovary | 0.6 |
| Thymus | 0.9 | Ovarian ca. OVCAR-3 | 2.9 |
| Spleen | 6.8 | Ovarian ca. OVCAR-4 | 0.7 |
| Lymph node | 6.0 | Ovarian ca. OVCAR-5 | 1.3 |
| Colorectal | 0.5 | Ovarian ca. OVCAR-8 | 1.1 |
| Stomach | 60.7 | Ovarian ca. IGROV-1 | 5.7 |
| Small intestine | 5.9 | Ovarian ca.* (ascites) SK-OV-3 | 3.9 |
| Colon ca. SW480 | 0.1 | Uterus | 2.4 |
| Colon ca.* (SW480 met) SW620 | 1.2 | Placenta | 1.8 |
| Colon ca. HT29 | 0.2 | Prostate | 2.4 |
| Colon ca. HCT-116 | 0.4 | Prostate ca.* (bone met) PC-3 | 0.3 |
| Colon ca. CaCo-2 | 1.1 | Testis | 4.2 |
| 83219 CC Well to Mod Diff | 2.7 | Melanoma Hs688(A).T | 0.2 |
| (ODO3866) | | | |
| Colon ca. HCC-2998 | 6.9 | Melanoma* (met) Hs688(B).T | 0.7 |
| Gastric ca.* (liver met) NCI- | 0.5 | Melanoma UACC-62 | 0.1 |
| N87 | | | |
| Bladder | 9.7 | Melanoma M14 | 0.0 |
| Trachea | 4.8 | Melanoma LOX IMVI | 0.0 |
| Kidney | 0.8 | Melanoma* (met) SK-MEL-5 | 0.0 |
| Kidney (fetal) | 0.4 | Adipose | 9.8 |

TABLE L
Panel 1.3D

| | Relative | | Relative |
|---|---|---|---|
| | Expression(%) | | Expression(%) |
| | 1.3dtm4554f_{—} | | 1.3dtm4554f_{—} |
| Tissue Name | ag2455 | Tissue Name | ag2455 |
| Liver adenocarcinoma | 0.0 | Kidney (fetal) | 0.0 |
| Pancreas | 1.4 | Renal ca. 786-0 | 0.0 |
| Pancreatic ca. CAPAN 2 | 0.0 | Renal ca. A498 | 0.0 |
| Adrenal gland | 6.0 | Renal ca. RXF 393 | 1.2 |
| Thyroid | 2.0 | Renal ca. ACHN | 0.0 |
| Salivary gland | 1.8 | Renal ca. UO-31 | 0.0 |
| Pituitary gland | 1.4 | Renal ca. TK-10 | 0.0 |
| Brain (fetal) | 5.1 | Liver | 0.0 |
| Brain (whole) | 32.3 | Liver (fetal) | 4.9 |
| Brain (amygdala) | 50.7 | Liver ca. (hepatoblast) HepG2 | 2.0 |
| Brain (cerebellum) | 84.1 | Lung | 10.3 |
| Brain (hippocampus) | 72.7 | Lung (fetal) | 0.2 |
| Brain (substantia nigra) | 7.2 | Lung ca. (small cell) LX-1 | 0.2 |
| Brain (thalamus) | 48.6 | Lung ca. (small cell) NCI-H69 | 0.8 |
| Cerebral Cortex | 90.8 | Lung ca. (s.cell var.) SHP-77 | 0.0 |
| Spinal cord | 39.5 | Lung ca. (large cell) NCI-H460 | 0.0 |
| CNS ca. (glio/astro) U87-MG | 1.5 | Lung ca. (non-sm. cell) A549 | 0.9 |
| CNS ca. (glio/astro) U-118-MG | 0.0 | Lung ca. (non-s.cell) NCI-H23 | 0.0 |
| CNS ca. (astro) SW1783 | 1.0 | Lung ca. (non-s.cell) HOP-62 | 0.0 |
| CNS ca.* (neuro; met) SK-N- | 0.5 | Lung ca. (non-s.cl) NCI-H522 | 1.2 |
| AS | | | |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 2.7 |
| CNS ca. (glio) SNB-19 | 3.1 | Mammary gland | 1.2 |
| CNS ca. (glio) U251 | 0.0 | Breast ca.* (pl. effusion) MCF-7 | 0.8 |
| CNS ca. (glio) SF-295 | 0.0 | Breast ca.* (pl.ef) MDA-MB- | 0.0 |
| | | 231 | |
| Heart (fetal) | 1.1 | Breast ca.* (pl. effusion) T47D | 1.9 |
| Heart | 0.0 | Breast ca. BT-549 | 0.0 |
| Fetal Skeletal | 2.5 | Breast ca. MDA-N | 0.0 |
| Skeletal muscle | 1.1 | Ovary | 0.4 |
| Bone marrow | 36.9 | Ovarian ca. OVCAR-3 | 2.6 |
| Thymus | 21.3 | Ovarian ca. OVCAR-4 | 0.0 |
| Spleen | 100.0 | Ovarian ca. OVCAR-5 | 1.1 |
| Lymph node | 29.3 | Ovarian ca. OVCAR-8 | 0.8 |
| Colorectal | 0.2 | Ovarian ca. IGROV-1 | 0.8 |
| Stomach | 0.6 | Ovarian ca.* (ascites) SK-OV-3 | 0.0 |
| Small intestine | 4.0 | Uterus | 3.0 |
| Colon ca. SW480 | 0.0 | Placenta | 9.2 |
| Colon ca.* (SW480 met) SW620 | 2.4 | Prostate | 2.0 |
| Colon ca. HT29 | 0.8 | Prostate ca.* (bone met) PC-3 | 0.0 |
| Colon ca. HCT-116 | 0.0 | Testis | 6.6 |
| Colon ca. CaCo-2 | 9.3 | Melanoma Hs688(A).T | 0.0 |
| 83219 CC Well to Mod Diff | 3.2 | Melanoma* (met) Hs688(B).T | 0.0 |
| (ODO3866) | | | |
| Colon ca. HCC-2998 | 0.9 | Melanoma UACC-62 | 0.0 |
| Gastric ca.* (liver met) NCI- | 0.0 | Melanoma M14 | 0.0 |
| N87 | | | |
| Bladder | 2.9 | Melanoma LOX IMVI | 0.0 |
| Trachea | 5.3 | Melanoma* (met) SK-MEL-5 | 0.0 |
| Kidney | 0.0 | Adipose | 0.0 |

TABLE M
Panel 2D

| | Relative | Relative |
|---|---|---|
| | Expression(%) | Expression(%) |
| | 2Dtm2328f_{—} | 2dtm4516f_{—} |
| Tissue Name | ag1388 | ag2455 |
| Normal Colon GENPAK 061003 | 5.9 | 17.6 |
| 83219 CC Well to Mod Diff (ODO3866) | 10.1 | 9.0 |
| 83220 CC NAT (ODO3866) | 3.2 | 18.8 |
| 83221 CC Gr.2 rectosigmoid (ODO3868) | 4.2 | 4.2 |
| 83222 CC NAT (ODO3868) | 10.9 | 2.7 |
| 83235 CC Mod Diff (ODO3920) | 0.0 | 4.0 |
| 83236 CC NAT (ODO3920) | 5.0 | 3.0 |
| 83237 CC Gr.2 ascend colon (ODO3921) | 9.9 | 7.2 |
| 83238 CC NAT (ODO3921) | 2.4 | 17.0 |
| 83241 CC from Partial Hepatectomy (ODO4309) | 5.2 | 0.0 |
| 83242 Liver NAT (ODO4309) | 0.0 | 0.0 |
| 87472 Colon mets to lung (OD04451-01) | 11.0 | 17.1 |
| 87473 Lung NAT (OD04451-02) | 23.8 | 20.2 |
| Normal Prostate Clontech A+ 6546-1 | 6.3 | 4.7 |
| 84140 Prostate Cancer (OD04410) | 11.2 | 12.6 |
| 84141 Prostate NAT (OD04410) | 0.0 | 11.9 |
| 87073 Prostate Cancer (OD04720-01) | 0.0 | 4.4 |
| 87074 Prostate NAT (OD04720-02) | 3.5 | 7.2 |
| Normal Lung GENPAK 061010 | 25.0 | 23.8 |
| 83239 Lung Met to Muscle (ODO4286) | 0.0 | 4.3 |
| 83240 Muscle NAT (ODO4286) | 8.5 | 0.0 |
| 84136 Lung Malignant Cancer (OD03126) | 8.5 | 11.1 |
| 84137 Lung NAT (OD03126) | 0.0 | 15.3 |
| 84871 Lung Cancer (OD04404) | 6.8 | 0.0 |
| 84872 Lung NAT (OD04404) | 15.0 | 18.2 |
| 84875 Lung Cancer (OD04565) | 2.8 | 14.8 |
| 84876 Lung NAT (OD04565) | 5.0 | 13.8 |
| 85950 Lung Cancer (OD04237-01) | 2.4 | 0.0 |
| 85970 Lung NAT (OD04237-02) | 6.4 | 0.0 |
| 83255 Ocular Mel Met to Liver (ODO4310) | 0.0 | 0.0 |
| 83256 Liver NAT (ODO4310) | 4.8 | 0.0 |
| 84139 Melanoma Mets to Lung (OD04321) | 2.7 | 0.0 |
| 84138 Lung NAT (OD04321) | 13.5 | 31.4 |
| Normal Kidney GENPAK 061008 | 0.5 | 4.7 |
| 83786 Kidney Ca, Nuclear grade 2 (OD04338) | 9.8 | 7.5 |
| 83787 Kidney NAT (OD04338) | 1.6 | 2.1 |
| 83788 Kidney Ca Nuclear grade 1/2 (OD04339) | 2.3 | 10.2 |
| 83789 Kidney NAT (OD04339) | 0.0 | 0.0 |
| 83790 Kidney Ca, Clear cell type (OD04340) | 12.0 | 10.4 |
| 83791 Kidney NAT (OD04340) | 2.9 | 10.0 |
| 83792 Kidney Ca, Nuclear grade 3 (OD04348) | 5.6 | 2.5 |
| 83793 Kidney NAT (OD04348) | 5.5 | 1.4 |
| 87474 Kidney Cancer (OD04622-01) | 33.0 | 21.9 |
| 87475 Kidney NAT (OD04622-03) | 5.0 | 5.5 |
| 85973 Kidney Cancer (OD04450-01) | 0.0 | 0.0 |
| 85974 Kidney NAT (OD04450-03) | 0.0 | 0.0 |
| Kidney Cancer Clontech 8120607 | 0.0 | 9.1 |
| Kidney NAT Clontech 8120608 | 5.3 | 2.6 |
| Kidney Cancer Clontech 8120613 | 0.0 | 0.0 |
| Kidney NAT Clontech 8120614 | 0.0 | 0.0 |
| Kidney Cancer Clontech 9010320 | 24.7 | 32.5 |
| Kidney NAT Clontech 9010321 | 8.4 | 0.0 |
| Normal Uterus GENPAK 061018 | 3.7 | 2.0 |
| Uterus Cancer GENPAK 064011 | 5.7 | 2.3 |
| Normal Thyroid Clontech A+ 6570-1 | 3.2 | 4.4 |
| Thyroid Cancer GENPAK 064010 | 3.1 | 2.4 |
| Thyroid Cancer INVITROGEN A302152 | 4.4 | 9.3 |
| Thyroid NAT INVITROGEN A302153 | 0.0 | 11.0 |
| Normal Breast GENPAK 061019 | 34.9 | 23.3 |
| 84877 Breast Cancer (OD04566) | 0.9 | 11.1 |
| 85975 Breast Cancer (OD04590-01) | 33.9 | 39.0 |
| 85976 Breast Cancer Mets (OD04590-03) | 92.0 | 76.8 |
| 87070 Breast Cancer Metastasis (OD04655-05) | 100.0 | 100.0 |
| GENPAK Breast Cancer 064006 | 0.0 | 12.2 |
| Breast Cancer Res. Gen. 1024 | 5.5 | 13.7 |
| Breast Cancer Clontech 9100266 | 0.9 | 2.6 |
| Breast NAT Clontech 9100265 | 0.0 | 5.2 |
| Breast Cancer INVITROGEN A209073 | 6.0 | 4.2 |
| Breast NAT INVITROGEN A2090734 | 4.6 | 12.6 |
| Normal Liver GENPAK 061009 | 2.7 | 0.0 |
| Liver Cancer GENPAK 064003 | 3.3 | 1.3 |
| Liver Cancer Research Genetics RNA 1025 | 9.0 | 2.3 |
| Liver Cancer Research Genetics RNA 1026 | 5.6 | 1.6 |
| Paired Liver Cancer Tissue Research Genetics RNA 6004-T | 6.0 | 3.6 |
| Paired Liver Tissue Research Genetics RNA 6004-N | 0.0 | 2.6 |
| Paired Liver Cancer Tissue Research Genetics RNA 6005-T | 3.2 | 2.1 |
| Paired Liver Tissue Research Genetics RNA 6005-N | 8.1 | 0.0 |
| Normal Bladder GENPAK 061001 | 11.8 | 17.9 |
| Bladder Cancer Research Genetics RNA 1023 | 7.2 | 1.9 |
| Bladder Cancer INVITROGEN A302173 | 6.0 | 2.7 |
| 87071 Bladder Cancer (OD04718-01) | 1.5 | 2.6 |
| 87072 Bladder Normal Adjacent (OD04718-03) | 11.3 | 1.9 |
| Normal Ovary Res. Gen. | 0.3 | 2.1 |
| Ovarian Cancer GENPAK 064008 | 8.4 | 1.6 |
| 87492 Ovary Cancer (OD04768-07) | 0.0 | 2.7 |
| 87493 Ovary NAT (OD04768-08) | 0.0 | 0.0 |
| Normal Stomach GENPAK 061017 | 6.6 | 13.2 |
| Gastric Cancer Clontech 9060358 | 2.1 | 4.8 |
| NAT Stomach Clontech 9060359 | 6.0 | 7.1 |
| Gastric Cancer Clontech 9060395 | 11.0 | 7.5 |
| NAT Stomach Clontech 9060394 | 3.6 | 9.9 |
| Gastric Cancer Clontech 9060397 | 3.1 | 0.0 |
| NAT Stomach Clontech 9060396 | 0.0 | 4.3 |
| Gastric Cancer GENPAK 064005 | 0.0 | 6.8 |

TABLE N
Panels 4D/4R

| | | | Relative |
|---|---|---|---|
| | Relative Expression(%) | | Expression(%) |
| | 4Dtm1781f_{—} | 4rtm1790f_{—} | 4Dx4tm4260f_{—} |
| Tissue Name | ag1388 | ag1388 | ag2455_a1 |
| 93768_Secondary Th1_anti-CD28/anti-CD3 | 2.6 | 2.9 | 5.3 |
| 93769_Secondary Th2_anti-CD28/anti-CD3 | 4.9 | 3.7 | 6.5 |
| 93770_Secondary Tr1_anti-CD28/anti-CD3 | 9.9 | 3.4 | 5.1 |
| 93573_Secondary Th1_resting day 4-6 in IL-2 | 12.0 | 12.2 | 6.8 |
| 93572_Secondary Th2_resting day 4-6 in IL-2 | 12.1 | 16.4 | 14.9 |
| 93571_Secondary Tr1_resting day 4-6 in IL-2 | 11.5 | 16.2 | 25.2 |
| 93568_primary Th1_anti-CD28/anti-CD3 | 5.0 | 6.2 | 1.8 |
| 93569_primary Th2_anti-CD28/anti-CD3 | 4.6 | 5.5 | 1.0 |
| 93570_primary Tr1_anti-CD28/anti-CD3 | 5.8 | 4.0 | 1.4 |
| 93565_primary Th1_resting dy 4-6 in IL-2 | 31.2 | 100.0 | 23.4 |
| 93566_primary Th2_resting dy 4-6 in IL-2 | 36.1 | 55.1 | 27.2 |
| 93567_primary Tr1_resting dy 4-6 in IL-2 | 22.1 | 4.9 | 28.0 |
| 93351_CD45RA CD4 lymphocyte_anti- | 0.0 | 0.9 | 2.4 |
| CD28/anti-CD3 | | | |
| 93352_CD45RO CD4 lymphocyte_anti- | 3.6 | 4.0 | 2.2 |
| CD28/anti-CD3 | | | |
| 93251_CD8 Lymphocytes_anti-CD28/anti-CD3 | 3.6 | 2.4 | 2.5 |
| 93353_chronic CD8 Lymphocytes 2ry_resting | 3.7 | 3.0 | 0.0 |
| dy 4-6 in IL-2 | | | |
| 93574_chronic CD8 Lymphocytes 2ry_activated | 3.7 | 3.6 | 3.4 |
| CD3/CD28 | | | |
| 93354_CD4_none | 7.3 | 11.6 | 17.4 |
| 93252_Secondary Th1/Th2/Tr1_anti-CD95 | 39.2 | 43.2 | 18.5 |
| CH11 | | | |
| 93103_LAK cells_resting | 6.4 | 4.5 | 5.1 |
| 93788_LAK cells_IL-2 | 9.4 | 8.2 | 5.3 |
| 93787_LAK cells_IL-2 + IL-12 | 2.5 | 7.5 | 2.7 |
| 93789_LAK cells_IL-2 + IFN gamma | 3.9 | 13.7 | 3.7 |
| 93790_LAK cells_IL-2 + IL-18 | 1.5 | 1.3 | 3.2 |
| 93104_LAK cells_PMA/ionomycin and IL-18 | 0.0 | 0.4 | 1.0 |
| 93578_NK Cells IL-2_resting | 10.0 | 9.2 | 10.6 |
| 93109_Mixed Lymphocyte Reaction_Two Way | 3.1 | 6.9 | 8.8 |
| MLR | | | |
| 93110_Mixed Lymphocyte Reaction_Two Way | 0.0 | 1.4 | 4.0 |
| MLR | | | |
| 93111_Mixed Lymphocyte Reaction_Two Way | 6.7 | 6.0 | 6.5 |
| MLR | | | |
| 93112_Mononuclear Cells (PBMCs) resting | 9.7 | 12.5 | 5.9 |
| 93113_Mononuclear Cells (PBMCs)_PWM | 5.0 | 12.2 | 2.7 |
| 93114_Mononuclear Cells (PBMCs)_PHA-L | 5.6 | 5.1 | 4.7 |
| 93249_Ramos (B cell)_none | 6.8 | 5.7 | 4.7 |
| 93250_Ramos (B cell)_ionomycin | 4.1 | 48.3 | 9.4 |
| 93349_B lymphocytes_PWM | 7.1 | 17.7 | 10.3 |
| 93350_B lymphoytes_CD40L and IL-4 | 21.8 | 2.7 | 36.9 |
| 92665_EOL-1 (Eosinophil)_dbcAMP | 100.0 | 65.1 | 100.0 |
| differentiated | | | |
| 93248_EOL-1 | 17.6 | 35.4 | 37.9 |
| (Eosinophil)_dbcAMP/PMAionomycin | | | |
| 93356_Dendritic Cells_none | 1.4 | 2.0 | 0.0 |
| 93355_Dendritic Cells_LPS 100 ng/ml | 0.0 | 0.0 | 2.5 |
| 93775_Dendritic Cells_anti-CD40 | 0.0 | 0.7 | 0.7 |
| 93774_Monocytes_resting | 10.5 | 23.2 | 18.7 |
| 93776_Monocytes_LPS 50 ng/ml | 2.6 | 6.2 | 3.0 |
| 93581_Macrophages_resting | 2.8 | 5.1 | 5.5 |
| 93582_Macrophages_LPS 100 ng/ml | 0.0 | 1.1 | 1.6 |
| 93098_HUVEC (Endothelial)_none | 0.0 | 0.1 | 0.0 |
| 93099_HUVEC (Endothelial)_starved | 0.0 | 0.0 | 0.0 |
| 93100_HUVEC (Endothelial)_IL-1b | 0.0 | 0.2 | 0.0 |
| 93779_HUVEC (Endothelial)_IFN gamma | 0.0 | 0.0 | 0.0 |
| 93102_HUVEC (Endothelial)_TNF alpha + IFN | 0.0 | 0.0 | 0.0 |
| gamma | | | |
| 93101_HUVEC (Endothelial)_TNF alpha + IL4 | 0.0 | 0.1 | 0.0 |
| 93781_HUVEC (Endothelial)_IL-11 | 0.0 | 0.0 | 0.0 |
| 93583_Lung Microvascular Endothelial | 0.0 | 0.0 | 0.0 |
| Cells_none | | | |
| 93584_Lung Microvascular Endothelial | 0.0 | 0.4 | 0.0 |
| Cells_TNFa (4 ng/ml) and IL1b (1 ng/ml) | | | |
| 92662_Microvascular Dermal | 0.0 | 0.4 | 0.0 |
| endothelium_none | | | |
| 92663_Microsvasular Dermal | 0.0 | 0.3 | 0.0 |
| endothelium_TNFa (4 ng/ml) and IL1b (1 ng/ml) | | | |
| 93773_Bronchial epithelium_TNFa (4 ng/ml) | 1.5 | 0.0 | 0.0 |
| and IL1b (1 ng/ml)** | | | |
| 93347_Small Airway Epithelium_none | 0.0 | 0.0 | 0.0 |
| 93348_Small Airway Epithelium_TNFa (4 | 0.0 | 0.2 | 0.7 |
| ng/ml) and IL1b (1 ng/ml) | | | |
| 92668_Coronery Artery SMC_resting | 1.3 | 0.0 | 0.0 |
| 92669_Coronery Artery SMC_TNFa (4 ng/ml) | 0.0 | 0.0 | 0.0 |
| and IL1b (1 ng/ml) | | | |
| 93107_astrocytes_resting | 0.0 | 0.5 | 0.0 |
| 93108_astrocytes_TNFa (4 ng/ml) and IL1b (1 ng/ml) | 0.0 | 0.0 | 0.0 |
| 92666_KU-812 (Basophil)_resting | 1.9 | 5.6 | 3.5 |
| 92667_KU-812 (Basophil)_PMA/ionoycin | 2.4 | 6.7 | 3.9 |
| 93579_CCD1106 (Keratinocytes)_none | 0.0 | 0.0 | 0.0 |
| 93580_CCD1106 (Keratinocytes)_TNFa and | 0.0 | 0.4 | 0.0 |
| IFNg** | | | |
| 93791_Liver Cirrhosis | 10.7 | 1.8 | 7.3 |
| 93792_Lupus Kidney | 2.5 | 2.6 | 1.5 |
| 93577_NCI-H292 | 0.0 | 4.5 | 3.8 |
| 93358_NCI-H292_IL-4 | 0.0 | 2.3 | 0.0 |
| 93360_NCI-H292_IL-9 | 1.3 | 0.4 | 0.5 |
| 93359_NCI-H292_IL-13 | 0.0 | 0.4 | 0.0 |
| 93357_NCI-H292_IFN gamma | 0.0 | 0.5 | 0.0 |
| 93777_HPAEC_- | 0.0 | 0.0 | 0.0 |
| 93778_HPAEC_IL-1 beta/TNA alpha | 0.0 | 0.1 | 0.4 |
| 93254_Normal Human Lung Fibroblast_none | 0.0 | 0.8 | 0.0 |
| 93253_Normal Human Lung Fibroblast_TNFa | 0.0 | 0.6 | 0.0 |
| (4 ng/ml) and IL-1b (1 ng/ml) | | | |
| 93257_Normal Human Lung Fibroblast_IL-4 | 0.0 | 0.0 | 0.0 |
| 93256_Normal Human Lung Fibroblast_IL-9 | 0.0 | 0.0 | 0.0 |
| 93255_Normal Human Lung Fibroblast_IL-13 | 0.0 | 0.0 | 0.0 |
| 93258_Normal Human Lung Fibroblast_IFN | 0.0 | 0.0 | 0.0 |
| gamma | | | |
| 93106_Dermal Fibroblasts CCD1070_resting | 0.0 | 0.2 | 0.0 |
| 93361_Dermal Fibroblasts CCD1070_TNF | 8.9 | 19.2 | 9.4 |
| alpha 4 ng/ml | | | |
| 93105_Dermal Fibroblasts CCD1070_IL-1 beta | 0.0 | 0.0 | 0.5 |
| 1 ng/ml | | | |
| 93772_dermal fibroblast_IFN gamma | 0.0 | 0.0 | 0.0 |
| 93771_dermal fibroblast_IL-4 | 0.0 | 0.0 | 0.0 |
| 93259_IBD Colitis 1** | 2.9 | 0.2 | 0.9 |
| 93260_IBD Colitis 2 | 1.5 | 1.1 | 2.4 |
| 93261_IBD Crohns | 1.4 | 0.5 | 0.0 |
| 735010_Colon_normal | 34.9 | 5.5 | 31.0 |
| 735019_Lung_none | 11.8 | 2.7 | 11.6 |
| 64028-1_Thymus_none | 1.5 | 0.4 | 0.9 |
| 64030-1_Kidney_none | 4.5 | 6.7 | 11.8 |

TABLE O
Panel CNSD.01

| | Relative | | Relative |
|---|---|---|---|
| | Expression (%) | | Expression (%) |
| | cns1x4tm6186f_{—} | | cns1x4tm6186f_{—} |
| Tissue Name | ag2455_a2 | Tissue Name | ag2455_a2 |
| 102633_BA4 Control | 9.1 | 102605_BA17 PSP | 1.3 |
| 102641_BA4 Control2 | 59.7 | 102612_BA17 PSP2 | 8.6 |
| 102625_BA4 Alzheimer's2 | 14.7 | 102637_Sub Nigra Control | 31.8 |
| 102649_BA4 Parkinson's | 20.1 | 102645_Sub Nigra Control2 | 35.7 |
| 102656_BA4 Parkinson's2 | 51.0 | 102629_Sub Nigra | 15.7 |
| | | Alzheimer's2 | |
| 102664_BA4 Huntington's | 5.6 | 102660_Sub Nigra Parkinson's2 | 29.9 |
| 102671_BA4 Huntington's2 | 29.6 | 102667_Sub Nigra | 50.1 |
| | | Huntington's | |
| 102603_BA4 PSP | 9.6 | 102674_Sub Nigra | 9.8 |
| | | Huntington's2 | |
| 102610_BA4 PSP2 | 22.2 | 102614_Sub Nigra PSP2 | 2.0 |
| 102588_BA4 Depression | 0.9 | 102592_Sub Nigra Depression | 0.0 |
| 102596_BA4 Depression2 | 8.8 | 102599_Sub Nigra Depression2 | 0.0 |
| 102634_BA7 Control | 13.6 | 102636_Glob Palladus Control | 43.8 |
| 102642_BA7 Control2 | 31.4 | 102644_Glob Palladus Control2 | 100.0 |
| 102626_BA7 Alzheimer's2 | 0.0 | 102620_Glob Palladus | 18.3 |
| | | Alzheimer's | |
| 102650_BA7 Parkinson's | 23.4 | 102628_Glob Palladus | 15.2 |
| | | Alzheimer's2 | |
| 102657_BA7 Parkinson's2 | 27.4 | 102652_Glob Palladus | 24.9 |
| | | Parkinson's | |
| 102665_BA7 Huntington's | 21.7 | 102659_Glob Palladus | 66.9 |
| | | Parkinson's2 | |
| 102672_BA7 Huntington's2 | 36.8 | 102606_Glob Palladus PSP | 45.4 |
| 102604_BA7 PSP | 7.7 | 102613_Glob Palladus PSP2 | 43.1 |
| 102611_BA7 PSP2 | 0.0 | 102591_Glob Palladus | 12.9 |
| | | Depression | |
| 102589_BA7 Depression | 9.0 | 102638_Temp Pole Control | 23.1 |
| 102632_BA9 Control | 3.7 | 102646_Temp Pole Control2 | 67.9 |
| 102640_BA9 Control2 | 30.4 | 102622_Temp Pole Alzheimer's | 2.5 |
| 102617_BA9 Alzheimer's | 0.0 | 102630_Temp Pole | 6.7 |
| | | Azheimer's2 | |
| 102624_BA9 Alzheimer's2 | 1.7 | 102653_Temp Pole Parkinson's | 39.7 |
| 102648_BA9 Parkinson's | 6.8 | 102661_Temp Pole | 12.8 |
| | | Parkinson's2 | |
| 102655_BA9 Parkinson's2 | 15.7 | 102668_Temp Pole | 26.1 |
| | | Huntington's | |
| 102663_BA9 Huntington's | 21.7 | 102607_Temp Pole PSP | 0.0 |
| 102670_BA9 Huntington's2 | 1.1 | 102615_Temp Pole PSP2 | 0.0 |
| 102602_BA9 PSP | 3.6 | 102600_Temp Pole | 4.8 |
| | | Depression2 | |
| 102609_BA9 PSP2 | 6.2 | 102639_Cing Gyr Control | 36.1 |
| 102587_BA9 Depression | 8.5 | 102647_Cing Gyr Control2 | 28.9 |
| 102595_BA9 Depression2 | 0.0 | 102623_Cing Gyr Alzheimer's | 7.0 |
| 102635_BA17 Control | 12.7 | 102631_Cing Gyr Alzheimer's2 | 0.0 |
| 102643_BA17 Control2 | 36.0 | 102654_Cing Gyr Parkinson's | 17.7 |
| 102627_BA17 Alzheimer's2 | 5.3 | 102662_Cing Gyr Parkinson's2 | 14.1 |
| 102651_BA17 Parkinson's | 23.5 | 102669_Cing Gyr Huntington's | 52.1 |
| 102658_BA17 Parkinson's2 | 18.3 | 102676_Cing Gyr | 8.5 |
| | | Huntington's2 | |
| 102666_BA17 Huntington's | 24.9 | 102608_Cing Gyr PSP | 0.0 |
| 102673_BA17 Huntington's2 | 6.8 | 102616_Cing Gyr PSP2 | 0.6 |
| 102590_BA17 Depression | 3.7 | 102594_Cing Gyr Depression | 5.0 |
| 102597_BA17 Depression2 | 6.6 | 102601_Cing Gyr Depression2 | 3.4 |

Panel 1.2 Summary: Ag1388 Expression of the NOV4 gene in the samples on this panel seems to be restricted, in large part, to normal tissues. The NOV4 gene is most highly expressed in a sample derived from cerebellum (CT 26). Expression of this gene is also prominent in stomach. Based upon this pattern of expression, the expression of this gene might be of use as a marker of cerebellar or stomach tissue.

Among CNS samples, the NOV4 gene is expressed in cerebellum, amygdala, hippocampus, thalamus, cerebral cortex and spinal cord. This result is consistent with what is observed in Panel 1.3D; please see below for summary of potential implications of the expression of this gene in the CNS.

The NOV4 gene encodes a type 1 membrane protein with several leucine-rich-repeat domains, indicating that this gene product may be involved in extracellular signalling and/or interactions with the extracellular matrix. Among metabolically relevant tissues, this gene is expressed at low but significant levels in the adrenal gland, thyroid, heart and liver. As a potential extracelular signalling molecule, the NOV4 gene product may serve as an antibody target for diseases involving any or all of these tissues.

Panel 1.3D Summary: Ag2455 Expression of the NOV4 gene in this panel is largely restricted to normal brain and normal lymphoid tissues. Highest expression of this gene is detected in spleen (CT=30), with lower but significant expression in lymph node, bone marrow and thymus. Thus, the expression of this gene might be useful as a marker of lymphoid tissue.

Moderate and roughly equivalent expression is also detected in several regions of the CNS including amygdala, cerebellum, substantia nigra, hippocampus, thalamus, cerebral cortex and spinal cord. In Drosophilia, the LRR region of axon guidance proteins has been shown to be critical for function (especially in axon repulsion) (Battye et al., Repellent signaling by Slit requires the leucine-rich repeats. J. Neurosci. 21: 4290-4298, 2001). Since the NOV4 gene encodes a leucine-rich-repeat protein that is expressed across all brain regions, it is an excellent candidate neuronal guidance protein for axons, dendrites and/or growth cones in general. Therefore, therapeutic modulation of the levels of this protein, or possible signaling via this protein, may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease).

Panel 2D Summary: Ag1388/Ag2455 Results from two experiments using different probe/primer sets are in good agreement. Strikingly, expression of the NOV4 gene is highest in two metastatic breast cancer samples (CT=31-32), and is also detectable in several other breast cancer samples. In addition, there appears to be a moderate association with overexpression of the NOV4 gene in kidney cancers when compared to their normal adjacent tissues, as 6 of 9 pairs show this pattern of expression. Thus, expression of this gene could be used as a marker for the detection of breast or kidney cancer. In addition, therapeutic down modulation of the NOV4 gene product, through the use of antibodies or small molecule drugs, may be useful for the treatment of breast or kidney cancer.

Panel 4D/4R Summary: Ag1388/Ag2455 Significant expression of the NOV4 gene is detected in bone marrow, spleen, and lymph node, as well as in the thymus in one experiment. These results are consistent with what is observed in Panel 1.3D. In addition, differential NOV4 gene expression is observed in the eosinophil cell line EOL-1 under resting conditions over that in EOL-1 cells stimulated by phorbol ester and ionomycin. Furthermore, unstimulated T lymphocytes (Th1, Th2, and Tr1) expressed this gene at higher levels than anti-CD28+anti-CD3-stimulated T cells. Thus, the NOV4 gene may be involved in both eosinophil and T lymphocyte function. Antibodies raised against the NOV4 protein that stimulate its activity may be useful in reduction of eosinophil activation and may therefore be useful therapeutic antibodies for asthma and allergy, and also as anti-inflammatory therapeutics for T cell-mediated autoimmune and inflammatory diseases. Furthermore, the isolated extracellular domain of the NOV4 protein may likewise function as a protein therapeutic in the treatment of asthma, emphysema, and allergy, as well as in other autoimmune and inflammatory diseases such as rheumatoid arthritis, inflammatory bowel disease, and psoriasis.

Panel CNSD.01 Summary: Ag2455 Among the samples on this panel, the NOV4 gene is most highly expressed in the globus palladus, a region of the basal ganglia involved in the control of movement; various inputs to the globus palladus are lost in Parkinson's disease and Huntington' disease. Since there is evidence that leucine-rich repeat proteins are critical in axonal guidance, the protein encoded by the NOV4 gene may be important in the treatment of Parkinson's and/or Huntington's disease by stimulating neuroregeneration and/or stem cell implantation for the establishment of connectivity. Likewise modulation of the activity of this protein may serve to slow or stop neurodegeneration in these diseases.

NOV5: CD81/Tetraspanin-Like

Expression of gene NOV5 (GM_{—}51624520_A1/dj1160k1_A1) was assessed using the primer-probe sets Ag2940, Ag610 and Ag1199, described in Tables P, Q, and R. Results of the RTQ-PCR runs are shown in Tables S, T, U, V and W.

TABLE P
Probe Name Ag2940

| | | | | Start | SEQ ID |
|---|---|---|---|---|---|
| Primers | Sequences | TM | Length | Position | NO: |
| Forward | 5′-CTGAGCTGCATGAAGTATCTGA-3′ | 58.3 | 22 | 15 | 98 |
| Probe | TET-5′-TCAATTTCTTCATATTTCTGGGCGGG-3′-TAMRA | 68.5 | 26 | 46 | 99 |
| Reverse | 5′-GTCCACCATGACCCAGATC-3′ | 59.7 | 19 | 110 | 100 |

TABLE Q
Probe Name Ag610

| | | | | Start | SEQ ID |
|---|---|---|---|---|---|
| Primers | Sequences | TM | Length | Position | NO: |
| Forward | 5′-GCACTACCAGGGCAATAACGA-3′ | | 21 | 373 | 101 |
| Probe | FAM-5′-ACGTCTTCTCTCCCACCTGGAACTCG-3′-TAMRA | | 26 | 399 | 102 |
| Reverse | 5′-GCAGCAACCAAATGTGATCATG-3′ | | 22 | 427 | 103 |

TABLE R
Probe Name Ag1199

| | | | | Start | SEQ ID |
|---|---|---|---|---|---|
| Primers | Sequences | TM | Length | Position | NO: |
| Forward | 5′-TGTTCATCCTGATCATCTTCCT-3′ | 58.6 | 22 | 270 | 104 |
| Probe | FAM-5′-AGCCATCCTGGCCTTCATCTTCAG-3′-TAMRA | 68.8 | 24 | 307 | 105 |
| Reverse | 5′-AGAATTCTCGGGTGAGATTTTC-3′ | 58.7 | 22 | 332 | 106 |

TABLE S
Panel 1.1

| | Relative | | Relative |
|---|---|---|---|
| | Expression (%) | | Expression (%) |
| | 1.1tm767f_{—} | | 1.1tm767f_{—} |
| Tissue Name | ag610 | Tissue Name | ag610 |
| Adipose | 1.8 | Renal ca. TK-10 | 12.0 |
| Adrenal gland | 30.6 | Renal ca. UO-31 | 8.0 |
| Bladder | 5.5 | Renal ca. RXF 393 | 5.1 |
| Brain (amygdala) | 1.7 | Liver | 8.5 |
| Brain (cerebellum) | 85.3 | Liver (fetal) | 3.7 |
| Brain (hippocampus) | 8.2 | Liver ca. (hepatoblast) HepG2 | 0.0 |
| Brain (substantia nigra) | 7.5 | Lung | 9.2 |
| Brain (thalamus) | 5.7 | Lung (fetal) | 13.0 |
| Cerebral Cortex | 2.6 | Lung ca (non-s.cell) HOP-62 | 15.3 |
| Brain (fetal) | 23.8 | Lung ca. (large cell) NCI-H460 | 0.0 |
| Brain (whole) | 6.9 | Lung ca. (non-s.cell) NCI-H23 | 1.3 |
| CNS ca. (glio/astro) U-118-MG | 0.0 | Lung ca. (non-s.cl) NCI-H522 | 4.6 |
| CNS ca. (astro) SF-539 | 0.7 | Lung ca. (non-sm. cell) A549 | 0.3 |
| CNS ca. (astro) SNB-75 | 1.2 | Lung ca. (s.cell var.) SHP-77 | 0.0 |
| CNS ca. (astro) SW1783 | 2.3 | Lung ca. (small cell) LX-1 | 0.0 |
| CNS ca. (glio) U251 | 0.0 | Lung ca. (small cell) NCI-H69 | 0.4 |
| CNS ca. (glio) SF-295 | 9.0 | Lung ca. (squam.) SW 900 | 0.2 |
| CNS ca. (glio) SNB-19 | 0.0 | Lung ca. (squam.) NCI-H596 | 0.6 |
| CNS ca. (glio/astro) U87-MG | 0.0 | Lymph node | 4.6 |
| CNS ca.* (neuro; met) SK-N- | 49.7 | Spleen | 3.3 |
| AS | | | |
| Mammary gland | 9.7 | Thymus | 1.0 |
| Breast ca. BT-549 | 0.0 | Ovary | 12.1 |
| Breast ca. MDA-N | 0.0 | Ovarian ca. IGROV-1 | 1.6 |
| Breast ca.* (pl. effusion) T47D | 0.0 | Ovarian ca. OVCAR-3 | 4.9 |
| Breast ca.* (pl. effusion) MCF-7 | 0.0 | Ovarian ca. OVCAR-4 | 0.5 |
| Breast ca.* (pl.ef) MDA-MB- | 0.0 | Ovarian ca. OVCAR-5 | 2.5 |
| 231 | | | |
| Small intestine | 17.6 | Ovarian ca. OVCAR-8 | 0.0 |
| Colorectal | 4.0 | Ovarian ca.* (ascites) SK-OV-3 | 8.8 |
| Colon ca. HT29 | 0.0 | Pancreas | 8.3 |
| Colon ca. CaCo-2 | 5.4 | Pancreatic ca. CAPAN 2 | 9.7 |
| Colon ca. HCT-15 | 0.0 | Pituitary gland | 6.5 |
| Colon ca. HCT-116 | 4.7 | Placenta | 15.8 |
| Colon ca. HCC-2998 | 0.0 | Prostate | 4.8 |
| Colon ca. SW480 | 0.0 | Prostate ca.* (bone met) PC-3 | 0.0 |
| Colon ca.* (SW480 | 0.0 | Salivary gland | 4.1 |
| met) SW620 | | | |
| Stomach | 9.9 | Trachea | 2.9 |
| Gastric ca.* (liver met) NCI- | 0.0 | Spinal cord | 7.2 |
| N87 | | | |
| Heart | 100.0 | Testis | 4.1 |
| Fetal Skeletal | 27.4 | Thyroid | 10.1 |
| Skeletal muscle | 16.6 | Uterus | 11.1 |
| Endothelial cells | 84.7 | Melanoma M14 | 0.0 |
| Heart (fetal) | 55.1 | Melanoma LOX IMVI | 0.0 |
| Kidney | 43.8 | Melanoma UACC-62 | 0.0 |
| Kidney (fetal) | 12.3 | Melanoma SK-MEL-28 | 0.0 |
| Renal ca. 786-0 | 0.0 | Melanoma* (met) SK-MEL-5 | 2.0 |
| Renal ca. A498 | 0.0 | Melanoma Hs688(A).T | 10.1 |
| Renal ca. ACHN | 2.2 | Melanoma* (met) Hs688(B).T | 3.7 |

TABLE T
Panel 1.2

| | Relative | | Relative |
|---|---|---|---|
| | Expression (%) | | Expression (%) |
| | 1.2tm1395f_{—} | | 1.2tm1395f_{—} |
| Tissue Name | ag1199 | Tissue Name | ag1199 |
| Endothelial cells | 19.6 | Renal ca. 786-0 | 0.0 |
| Heart (fetal) | 97.3 | Renal ca. A498 | 0.0 |
| Pancreas | 0.3 | Renal ca. RXF 393 | 7.7 |
| Pancreatic ca. CAPAN 2 | 19.3 | Renal ca. ACHN | 1.0 |
| Adrenal Gland (new lot*) | 92.0 | Renal ca. UO-31 | 2.5 |
| Thyroid | 1.4 | Renal ca. TK-10 | 2.6 |
| Salivary gland | 2.5 | Liver | 9.7 |
| Pituitary gland | 2.6 | Liver (fetal) | 6.0 |
| Brain (fetal) | 16.6 | Liver ca. (hepatoblast) HepG2 | 0.0 |
| Brain (whole) | 7.6 | Lung | 13.4 |
| Brain (amygdala) | 5.1 | Lung (fetal) | 8.9 |
| Brain (cerebellum) | 51.4 | Lung ca. (small cell) LX-1 | 0.0 |
| Brain (hippocampus) | 16.3 | Lung ca. (small cell) NCI-H69 | 0.0 |
| Brain (thalamus) | 3.7 | Lung ca. (s.cell var.) SHP-77 | 0.0 |
| Cerebral Cortex | 5.6 | Lung ca. (large cell) NCI-H460 | 0.0 |
| Spinal cord | 7.7 | Lung ca. (non-sm. cell) A549 | 0.0 |
| CNS ca. (glio/astro) U87-MG | 0.0 | Lung ca. (non-s.cell) NCI-H23 | 0.4 |
| CNS ca. (glio/astro) U-118-MG | 0.0 | Lung ca (non-s.cell) HOP-62 | 2.2 |
| CNS ca. (astro) SW1783 | 1.6 | Lung ca. (non-s.cl) NCI-H522 | 3.4 |
| CNS ca.* (neuro; met) SK-N- | 20.7 | Lung ca. (squam.) SW 900 | 0.0 |
| AS | | | |
| CNS ca. (astro) SF-539 | 0.1 | Lung ca. (squam) NCI-H596 | 0.0 |
| CNS ca. (astro) SNB-75 | 0.5 | Mammary gland | 7.1 |
| CNS ca. (glio) SNB-19 | 0.0 | Breast ca.* (pl. effusion) MCF-7 | 0.0 |
| CNS ca. (glio) U251 | 0.0 | Breast ca.* (pl.ef) MDA-MB- | 0.0 |
| | | 231 | |
| CNS ca. (glio) SF-295 | 3.0 | Breast ca.* (pl. effusion) T47D | 0.0 |
| Heart | 100.0 | Breast ca. BT-549 | 0.0 |
| Skeletal Muscle (new lot*) | 4.9 | Breast ca. MDA-N | 0.0 |
| Bone marrow | 1.4 | Ovary | 22.4 |
| Thymus | 0.9 | Ovarian ca. OVCAR-3 | 1.2 |
| Spleen | 3.0 | Ovarian ca. OVCAR-4 | 0.2 |
| Lymph node | 6.9 | Ovarian ca. OVCAR-5 | 0.3 |
| Colorectal | 5.4 | Ovarian ca. OVCAR-8 | 0.0 |
| Stomach | 12.9 | Ovarian ca. IGROV-1 | 0.3 |
| Small intestine | 20.2 | Ovarian ca.* (ascites) SK-OV-3 | 4.9 |
| Colon ca. SW480 | 0.0 | Uterus | 10.4 |
| Colon ca.* (SW480 met) SW620 | 0.0 | Placenta | 17.0 |
| Colon ca. HT29 | 0.0 | Prostate | 7.1 |
| Colon ca. HCT-116 | 0.9 | Prostate ca.* (bone met) PC-3 | 0.0 |
| Colon ca. CaCo-2 | 1.8 | Testis | 2.7 |
| 83219 CC Well to Mod Diff | 3.3 | Melanoma Hs688(A).T | 8.4 |
| (ODO3866) | | | |
| Colon ca. HCC-2998 | 0.0 | Melanoma* (met) Hs688(B).T | 1.8 |
| Gastric ca.* (liver met) NCI- | 0.0 | Melanoma UACC-62 | 0.0 |
| N87 | | | |
| Bladder | 5.5 | Melanoma M14 | 0.0 |
| Trachea | 3.4 | Melanoa LOX IMVI | 0.0 |
| Kidney | 4.9 | Melanoma* (met) SK-MEL-5 | 2.4 |
| Kidney (fetal) | 21.3 | Adipose | 16.0 |

TABLE U
Panel 1.3D

| | Relative | | Relative |
|---|---|---|---|
| | Expression (%) | | Expression (%) |
| | 1.3dx4tm5354f_{—} | | 1.3dx4tm5354f_{—} |
| Tissue Name | ag610_b1 | Tissue Name | ag610_b1 |
| Liver adenocarcinoma | 0.5 | Kidney (fetal) | 5.6 |
| Pancreas | 2.2 | Renal ca. 786-0 | 0.0 |
| Pancreatic ca. CAPAN 2 | 44.6 | Renal ca. A498 | 10.9 |
| Adrenal gland | 44.6 | Renal ca. RXF 393 | 35.9 |
| Thyroid | 15.3 | Renal ca. ACHN | 1.9 |
| Salivary gland | 1.1 | Renal ca. UO-31 | 2.9 |
| Pituitary gland | 5.9 | Renal ca. TK-10 | 2.7 |
| Brain (fetal) | 38.5 | Liver | 3.8 |
| Brain (whole) | 29.4 | Liver (fetal) | 13.6 |
| Brain (amygdala) | 9.9 | Liver ca. (hepatoblast) HepG2 | 0.0 |
| Brain (cerebellum) | 100.0 | Lung | 24.5 |
| Brain (hippocampus) | 31.7 | Lung (fetal) | 18.2 |
| Brain (substantia nigra) | 3.8 | Lung ca. (small cell) LX-1 | 0.0 |
| Brain (thalamus) | 29.2 | Lung ca. (small cell) NCI-H69 | 0.0 |
| Cerebral Cortex | 2.4 | Lung ca. (s.cell var.) SHP-77 | 0.0 |
| Spinal cord | 18.8 | Lung ca. (large cell) NCI-H460 | 0.5 |
| CNS ca. (glio/astro) U87-MG | 0.0 | Lung ca. (non-sm. cell) A549 | 0.2 |
| CNS ca. (glio/astro) U-118-MG | 0.0 | Lung ca. (non-s.cell) NCI-H23 | 4.2 |
| CNS ca. (astro) SW1783 | 9.8 | Lung ca. (non-s.cell) HOP-62 | 3.2 |
| CNS ca.* (neuro; met) SK-N- | 43.8 | Lung ca. (non-s.cl) NCI-H522 | 0.0 |
| AS | | | |
| CNS ca. (astro) SF-539 | 3.2 | Lung ca. (squam.) SW 900 | 0.0 |
| CNS ca. (astro) SNB-75 | 17.5 | Lung ca. (squam.) NCI-H596 | 0.2 |
| CNS ca. (glio) SNB-19 | 0.4 | Mammary gland | 15.9 |
| CNS ca. (glio) U251 | 0.3 | Breast ca.* (pl. effusion) MCF-7 | 0.0 |
| CNS ca. (glio) SF-295 | 8.1 | Breast ca.* (pl.ef) MDA-MB- | 0.0 |
| | | 231 | |
| Heart (fetal) | 58.6 | Breast ca.* (pl. effusion) T47D | 0.0 |
| Heart | 61.4 | Breast ca. BT-549 | 0.0 |
| Fetal Skeletal | 32.1 | Breast ca. MDA-N | 0.0 |
| Skeletal muscle | 18.1 | Ovary | 12.9 |
| Bone marrow | 1.6 | Ovarian ca. OVCAR-3 | 2.0 |
| Thymus | 3.5 | Ovarian ca. OVCAR-4 | 1.0 |
| Spleen | 13.5 | Ovarian ca. OVCAR-5 | 1.2 |
| Lymph node | 20.9 | Ovarian ca. OVCAR-8 | 0.0 |
| Colorectal | 7.0 | Ovarian ca. IGROV-1 | 0.6 |
| Stomach | 17.8 | Ovarian ca.* (ascites) SK-OV-3 | 9.1 |
| Small intestine | 59.9 | Uterus | 75.2 |
| Colon ca. SW480 | 0.0 | Placenta | 18.5 |
| Colon ca.* (SW480 met) SW620 | 0.0 | Prostate | 10.6 |
| Colon ca. HT29 | 0.0 | Prostate ca.* (bone met) PC-3 | 0.0 |
| Colon ca. HCT-116 | 4.7 | Testis | 10.8 |
| Colon ca. CaCo-2 | 3.3 | Melanoma Hs688(A).T | 9.2 |
| 83219 CC Well to Mod Diff | 8.0 | Melanoma* (met) Hs688(B).T | 1.7 |
| (ODO3866) | | | |
| Colon ca. HCC-2998 | 0.0 | Melanoma UACC-62 | 0.0 |
| Gastric ca.* (liver met) NCI- | 0.7 | Melanoma M14 | 0.0 |
| N87 | | | |
| Bladder | 1.5 | Melanoma LOX IMVI | 0.0 |
| Trachea | 6.8 | Melanoma* (met) SK-MEL-5 | 3.7 |
| Kidney | 15.4 | Adipose | 7.8 |

TABLE V
Panel 2D

| | Relative | | Relative |
|---|---|---|---|
| | Expression (%) | | Expression (%) |
| | 2dtm5571f_{—} | | 2dtm5571f_{—} |
| Tissue Name | ag610 | Tissue Name | ag610 |
| Normal Colon GENPAK | 59.5 | Kidney NAT Clontech | 33.4 |
| 061003 | | 8120608 | |
| 83219 CC Well to Mod Diff | 10.2 | Kidney Cancer Clontech | 14.2 |
| (ODO3866) | | 8120613 | |
| 83220 CC NAT (ODO3866) | 15.2 | Kidney NAT Clontech | 70.7 |
| | | 8120614 | |
| 83221 CC Gr.2 rectosigmoid | 5.0 | Kidney Cancer Clontech | 27.9 |
| (ODO3868) | | 9010320 | |
| 83222 CC NAT (ODO3868) | 21.8 | Kidney NAT Clontech | 76.8 |
| | | 9010321 | |
| 83235 CC Mod Diff | 14.3 | Normal Uterus GENPAK | 14.7 |
| (ODO3920) | | 061018 | |
| 83236 CC NAT (ODO3920) | 21.0 | Uterus Cancer GENPAK | 33.2 |
| | | 064011 | |
| 83237 CC Gr.2 ascend colon | 9.8 | Normal Thyroid Clontech A+ | 19.2 |
| (ODO3921) | | 6570-1 | |
| 83238 CC NAT (ODO3921) | 12.9 | Thyroid Cancer GENPAK | 3.2 |
| | | 064010 | |
| 83241 CC from Partial | 12.3 | Thyroid Cancer INVITROGEN | 3.5 |
| Hepatectomy (ODO4309) | | A302152 | |
| 83242 Liver NAT (ODO4309) | 7.5 | Thyroid NAT INVITROGEN | 6.9 |
| | | A302153 | |
| 87472 Colon mets to lung | 3.2 | Normal Breast GENPAK | 12.7 |
| (OD04451-01) | | 061019 | |
| 87473 Lung NAT (OD04451- | 7.6 | 84877 Breast Cancer | 6.7 |
| 02) | | (OD04566) | |
| Normal Prostate Clontech A+ | 26.8 | 85975 Breast Cancer | 12.0 |
| 6546-1 | | (OD04590-01) | |
| 84140 Prostate Cancer | 12.5 | 85976 Breast Cancer Mets | 26.8 |
| (OD04410) | | (OD04590-03) | |
| 84141 Prostate NAT | 28.9 | 87070 Breast Cancer | 7.1 |
| (OD04410) | | Metastasis (OD04655-05) | |
| 87073 Prostate Cancer | 11.3 | GENPAK Breast Cancer | 4.2 |
| (OD04720-01) | | 064006 | |
| 87074 Prostate NAT | 36.6 | Breast Cancer Res. Gen. 1024 | 8.4 |
| (OD04720-02) | | | |
| Normal Lung GENPAK | 37.6 | Breast Cancer Clontech | 12.1 |
| 061010 | | 9100266 | |
| 83239 Lung Met to Muscle | 5.1 | Breast NAT Clontech 9100265 | 9.9 |
| (ODO4286) | | | |
| 83240 Muscle NAT | 8.1 | Breast Cancer INVITROGEN | 19.1 |
| (ODO4286) | | A209073 | |
| 84136 Lung Malignant Cancer | 9.8 | Breast NAT INVITROGEN | 6.3 |
| (OD03126) | | A2090734 | |
| 84137 Lung NAT (OD03126) | 43.5 | Normal Liver GENPAK | 1.7 |
| | | 061009 | |
| 84871 Lung Cancer (OD04404) | 44.4 | Liver Cancer GENPAK 064003 | 4.1 |
| 84872 Lung NAT (OD04404) | 13.7 | Liver Cancer Research | 4.3 |
| | | Genetics RNA 1025 | |
| 84875 Lung Cancer (OD04565) | 7.1 | Liver Cancer Research | 10.4 |
| | | Genetics RNA 1026 | |
| 84876 Lung NAT (OD04565) | 9.4 | Paired Liver Cancer Tissue | 5.1 |
| | | Research Genetics RNA 6004-T | |
| 85950 Lung Cancer (OD04237- | 5.3 | Paired Liver Tissue Research | 1.6 |
| 01) | | Genetics RNA 6004-N | |
| 85970 Lung NAT (OD04237- | 36.9 | Paired Liver Cancer Tissue | 9.1 |
| 02) | | Research Genetics RNA 6005-T | |
| 83255 Ocular Mel Met to Liver | 0.6 | Paired Liver Tissue Research | 3.7 |
| (ODO4310) | | Genetics RNA 6005-N | |
| 83256 Liver NAT (ODO4310) | 6.1 | Normal Bladder GENPAK | 3.2 |
| | | 061001 | |
| 84139 Melanoma Mets to Lung | 0.5 | Bladder Cancer Research | 3.6 |
| (OD04321) | | Genetics RNA 1023 | |
| 84138 Lung NAT (OD04321) | 51.4 | Bladder Cancer INVITROGEN | 2.9 |
| | | A302173 | |
| Normal Kidney GENPAK | 29.1 | 87071 Bladder Cancer | 5.5 |
| 061008 | | (OD04718-01) | |
| 83786 Kidney Ca. Nuclear | 41.8 | 87072 Bladder Normal | 46.0 |
| grade 2 (OD04338) | | Adjacent (OD04718-03) | |
| 83787 Kidney NAT (OD04338) | 29.5 | Normal Ovary Res. Gen. | 13.5 |
| 83788 Kidney Ca Nuclear | 7.1 | Ovarian Cancer GENPAK | 20.7 |
| grade 1/2 (OD04339) | | 064008 | |
| 83789 Kidney NAT (OD04339) | 46.7 | 87492 Ovary Cancer | 2.3 |
| | | (OD04768-07) | |
| 83790 Kidney Ca. Clear cell | 100.0 | 87493 Ovary NAT (OD04768- | 18.3 |
| type (OD04340) | | 08) | |
| 83791 Kidney NAT (OD04340) | 22.7 | Normal Stomach GENPAK | 47.0 |
| | | 061017 | |
| 83792 Kidney Ca, Nuclear | 4.6 | Gastric Cancer Clontech | 28.3 |
| grade 3 (OD04348) | | 9060358 | |
| 83793 Kidney NAT (OD04348) | 11.8 | NAT Stomach Clontech | 10.5 |
| | | 9060359 | |
| 87474 Kidney Cancer | 26.6 | Gastric Cancer Clontech | 43.5 |
| (OD04622-01) | | 9060395 | |
| 87475 Kidney NAT (OD04622- | 5.7 | NAT Stomach Clontech | 16.4 |
| 03) | | 9060394 | |
| 85973 Kidney Cancer | 32.8 | Gastric Cancer Clontech | 12.7 |
| (OD04450-01) | | 9060397 | |
| 85974 Kidney NAT (OD04450- | 12.9 | NAT Stomach Clontech | 4.5 |
| 03) | | 9060396 | |
| Kidney Cancer Clontech | 17.4 | Gastric Cancer GENPAK | 16.7 |
| 8120607 | | 064005 | |

TABLE W
Panel 4D

| | Relative | Relative | Relative |
|---|---|---|---|
| | Expression (%) | Expression (%) | Expression (%) |
| | 4dtm5412t_ag | 4dx4tm5136f_{—} | 4Dtm2029f_ag |
| Tissue Name | 2940 | ag610_b1 | 1199 |
| 93768_Secondary Th1_anti-CD28/anti-CD3 | 0.7 | 0.5 | 0.0 |
| 93769_Secondary Th2_anti-CD28/anti-CD3 | 0.5 | 0.5 | 0.8 |
| 93770_Secondary Tr1_anti-CD28/anti-CD3 | 0.4 | 0.4 | 0.5 |
| 93573_Secondary Th1_resting day 4-6 in IL-2 | 7.1 | 8.3 | 12.0 |
| 93572_Secondary Th2_resting day 4-6 in IL-2 | 9.7 | 6.8 | 9.7 |
| 93571_Secondary Tr1_resting day 4-6 in IL-2 | 14.0 | 9.1 | 8.2 |
| 93568_primary Th1_anti-CD28/anti-CD3 | 0.9 | 0.3 | 1.0 |
| 93569_primary Th2_anti-CD28/anti-CD3 | 0.7 | 0.6 | 1.2 |
| 93570_primary Tr1_anti-CD28/anti-CD3 | 0.5 | 0.5 | 0.8 |
| 93565_primary Th1_resting dy 4-6 in IL-2 | 39.5 | 52.7 | 37.6 |
| 93566_primary Th2_resting dy 4-6 in IL-2 | 12.6 | 15.7 | 14.3 |
| 93567_primary Tr1_resting dy 4-6 in IL-2 | 13.7 | 15.6 | 13.4 |
| 93351_CD45RA CD4 lymphocyte_anti- | 0.5 | 0.6 | 0.4 |
| CD28/anti-CD3 | | | |
| 93352_CD45RO CD4 lymphocyte_anti- | 0.9 | 1.6 | 1.5 |
| CD28/anti-CD3 | | | |
| 93251_CD8 Lymphocytes_anti-CD28/anti-CD3 | 0.7 | 0.2 | 0.7 |
| 93353_chronic CD8 Lymphocytes 2ry_resting | 0.7 | 1.9 | 1.3 |
| dy 4-6 in IL-2 | | | |
| 93574_chronic CD8 Lymphocytes 2ry_activated | 0.8 | 0.4 | 1.0 |
| CD3/CD28 | | | |
| 93354_CD4_none | 10.4 | 9.4 | 8.2 |
| 93252_Secondary Th1/Th2/Tr1_anti-CD95 | 6.4 | 13.7 | 9.3 |
| CH11 | | | |
| 93103_LAK cells_resting | 1.0 | 2.0 | 3.3 |
| 93788_LAK cells_IL-2 | 0.8 | 0.7 | 1.5 |
| 93787_LAK cells_IL-2 + IL-12 | 0.5 | 1.4 | 1.5 |
| 93789_LAK cells_IL-2 + IFN gamma | 1.7 | 2.5 | 2.3 |
| 93790_LAK cells_IL-2 + IL-18 | 2.2 | 1.4 | 1.6 |
| 93104_LAK cells_PMA/ionomycin and IL-18 | 0.4 | 0.5 | 0.4 |
| 93578_NK Cells IL-2_resting | 1.4 | 0.6 | 0.6 |
| 93109_Mixed Lymphocyte Reaction_Two Way | 1.7 | 1.2 | 0.9 |
| MLR | | | |
| 93110_Mixed Lymphocyte Reaction_Two Way | 1.4 | 0.7 | 0.6 |
| MLR | | | |
| 93111_Mixed Lymphocyte Reaction_Two Way | 1.0 | 0.2 | 0.4 |
| MLR | | | |
| 93112_Mononuclear Cells (PBMCs)_resting | 7.7 | 6.0 | 7.1 |
| 93113_Mononuclear Cells (PBMCs)_PWM | 1.9 | 2.3 | 2.1 |
| 93114_Mononuclear Cells (PBMCs)_PHA-L | 1.7 | 3.6 | 3.3 |
| 93249_Ramos (B cell)_none | 0.0 | 0.0 | 0.0 |
| 93250_Ramos (B cell)_ionomycin | 0.0 | 0.0 | 0.0 |
| 93349_B lymphocytes_PWM | 0.9 | 2.1 | 1.1 |
| 93350_B lymphoytes_CD40L and IL-4 | 1.1 | 0.7 | 0.1 |
| 92665_EOL-1 (Eosinophil)_dbcAMP | 0.0 | 0.0 | 0.0 |
| differentiated | | | |
| 93248_EOL-1 | 0.0 | 0.0 | 0.0 |
| (Eosinophil)_dbcAMP/PMAionomycin | | | |
| 93356_Dendritic Cells_none | 0.0 | 0.0 | 0.1 |
| 93355_Dendritic Cells_LPS 100 ng/ml | 0.0 | 0.0 | 0.0 |
| 93775_Dendritic Cells_anti-CD40 | 0.0 | 0.0 | 0.0 |
| 93774_Monocytes_resting | 0.0 | 0.3 | 0.0 |
| 93776_Monocytes_LPS 50 ng/ml | 0.0 | 0.0 | 0.0 |
| 93581_Macrophages_resting | 0.1 | 0.4 | 0.2 |
| 93582_Macrophages_LPS 100 ng/ml | 0.0 | 0.0 | 0.0 |
| 93098_HUVEC (Endothelial)_none | 24.8 | 25.0 | 18.2 |
| 93099_HUVEC (Endothelial)_starved | 51.4 | 70.2 | 51.0 |
| 93100_HUVEC (Endothelial)_IL-1b | 21.5 | 24.4 | 16.8 |
| 93779_HUVEC (Endothelial)_IFN gamma | 61.6 | 36.6 | 37.9 |
| 93102_HUVEC (Endothelial)_TNF alpha + IFN | 6.9 | 6.6 | 2.3 |
| gamma | | | |
| 93101_HUVEC (Endothelial)_TNF alpha + IL4 | 6.9 | 4.8 | 3.1 |
| 93781_HUVEC (Endothelial)_IL-11 | 35.8 | 32.6 | 32.3 |
| 93583_Lung Microvascular Endothelial | 100.0 | 89.1 | 50.7 |
| Cells_none | | | |
| 93584_Lung Microvascular Endothelial | 27.5 | 38.0 | 16.4 |
| Cells_TNFa (4 ng/ml) and IL1b (1 ng/ml) | | | |
| 92662_Microvascular Dermal | 94.6 | 100.0 | 100.0 |
| endothelium_none | | | |
| 92663_Microsvasular Dermal | 32.5 | 45.0 | 33.2 |
| endothelium_TNFa (4 ng/ml) and IL1b (1 ng/ml) | | | |
| 93773_Bronchial epithelium_TNFa (4 ng/ml) | 0.0 | 0.0 | 0.2 |
| and IL1b (1 ng/ml)** | | | |
| 93347_Small Airway Epithelium_none | 0.0 | 0.0 | 0.0 |
| 93348_Small Airway Epithelium_TNFa (4 ng/ml) | 0.3 | 0.1 | 0.2 |
| and IL1b (1 ng/ml) | | | |
| 92668_Coronery Artery SMC_resting | 0.0 | 0.2 | 0.0 |
| 92669_Coronery Artery SMC_TNFa (4 ng/ml) | 0.0 | 0.0 | 0.0 |
| and IL1b (1 ng/ml) | | | |
| 93107_astrocytes_resting | 10.5 | 11.6 | 4.7 |
| 93108_astrocytes_TNFa (4 ng/ml) and IL1b (1 ng/ml) | 9.2 | 9.0 | 7.2 |
| 92666_KU-812 (Basophil)_resting | 0.2 | 1.9 | 1.1 |
| 92667_KU-812 (Basophil)_PMA/ionoycin | 3.2 | 4.6 | 2.2 |
| 93579_CCD1106 (Keratinocytes)_none | 0.0 | 0.0 | 0.0 |
| 93580_CCD1106 (Keratinocytes)_TNFa and | 0.0 | 0.0 | 0.4 |
| IFNg** | | | |
| 93791_Liver Cirrhosis | 1.1 | 1.4 | 0.5 |
| 93792_Lupus Kidney | 2.2 | 2.9 | 9.0 |
| 93577_NCI-H292 | 1.0 | 1.1 | 1.0 |
| 93358_NCI-H292_IL-4 | 0.9 | 1.9 | 1.2 |
| 93360_NCI-H292_IL-9 | 0.5 | 1.7 | 1.0 |
| 93359_NCI-H292_IL-13 | 1.7 | 1.1 | 1.0 |
| 93357_NCI-H292_IFN gamma | 0.9 | 1.4 | 1.1 |
| 93777_HPAEC_- | 27.2 | 21.4 | 21.6 |
| 93778_HPAEC_IL-1 beta/TNA alpha | 16.8 | 8.9 | 8.2 |
| 93254_Normal Human Lung Fibroblast_none | 0.0 | 0.0 | 0.0 |
| 93253_Normal Human Lung Fibroblast_TNFa | 0.0 | 0.0 | 0.0 |
| (4 ng/ml) and IL-1b (1 ng/ml) | | | |
| 93257_Normal Human Lung Fibroblast_IL-4 | 0.0 | 0.1 | 0.0 |
| 93256_Normal Human Lung Fibroblast_IL-9 | 0.0 | 0.0 | 0.1 |
| 93255_Normal Human Lung Fibroblast_IL-13 | 0.0 | 0.2 | 0.0 |
| 93258_Normal Human Lung Fibroblast_IFN | 0.0 | 0.0 | 0.0 |
| gamma | | | |
| 93106_Dermal Fibroblasts CCD1070_resting | 1.3 | 2.1 | 1.6 |
| 93361_Dermal Fibroblasts CCD1070_TNF | 7.3 | 10.5 | 6.8 |
| alpha 4 ng/ml | | | |
| 93105_Dermal Fibroblasts CCD1070_IL-1 beta | 0.8 | 0.7 | 0.7 |
| 1 ng/ml | | | |
| 93772_dermal fibroblast_IFN gamma | 0.0 | 0.0 | 0.1 |
| 93771_dermal fibroblast_IL-4 | 0.7 | 0.2 | 0.3 |
| 93259_IBD Colitis 1** | 7.3 | 8.1 | 7.9 |
| 93260_IBD Colitis 2 | 2.0 | 1.5 | 0.5 |
| 93261_IBD Crohns | 4.1 | 2.5 | 2.2 |
| 735010_Colon_normal | 33.7 | 26.9 | 15.0 |
| 735019_Lung_none | 52.8 | 62.1 | 33.9 |
| 64028-1_Thymus_none | 48.6 | 41.9 | 54.0 |
| 64030-1_Kidney_none | 2.4 | 3.4 | 3.9 |

Panel 1.1 Summary: Ag610 The NOV5 gene is highly expressed in a number of samples on this panel. Highest expression is detected in adult heart (CT value=22.7). This observation suggests that the NOV5 gene may play a role in heart homeostasis. Thus, therapeutic modulation of the expression of this gene might be useful in the treatment of heart diseases, including cardiomyopathy, atherosclerosis, hypertension, congenital heart defects, aortic stenosis, atrial septal defect (asd), atrioventricular (a-v) canal defect, ductus arteriosus, pulmonary stenosis, subaortic stenosis, ventricular septal defect (vsd), and valve diseases, or may aid recovery after damage to the heart.

Expression of the NOV5 gene is high in many regions of the brain, including the amygdala, thalamus, cerebellum, and cerebral cortex, with highest expression in the cerebellum (CT value=22.9). This observation suggests that the NOV5 gene may be involved in normal brain function and that disregulation of its expression may play a role in neurological diseases (see Panel 1.2 Summary for further discussion).

This gene is also moderately expressed in adrenal gland, pituitary gland, thyroid, skeletal muscle, liver, and pancreas. Expression in the metabolic tissues skeletal muscle, liver and pancreas suggest that the NOV5 gene may be involved in metabolic control processes and serve as a drug target for metabolic diseases, including obesity and diabetes. In addition, this gene may play a role in normal neuroendocrine function and disregulation may lead to disease.

In general, expression of this gene is associated with normal tissues but not with cancer cell lines. Interestingly, the gene is expressed to very high levels in normal mammary gland (CT value=26) but appears to be absent in 5/5 breast cancer cell lines. The NOV5 gene is also relatively under expressed in several CNS cancer cell lines relative to the normal brain. Therefore, the NOV5 gene product has potential utility as a protein therapeutic in the treatment of breast and CNS cancers. Please note that expression in adipose is skewed by the presence of genomic DNA contamination in this sample.

Panel 1.2 Summary: Ag1199 Expression of the NOV5 gene is high to moderate in many samples on this panel. However, gene expression is predominantly associated with normal tissues when compared to cell lines. The NOV5 gene is most highly expressed in adult and fetal heart tissue (CT=24). In addition, there appears to be high-level expression in adrenal gland. Based upon this expression profile, the expression of the NOV5 gene could be used as a marker of heart tissue or adrenal gland. Furthermore, therapeutic modulation of the activity of this gene product, through the application of the protein itself, or through the use of small molecule drugs or antibodies, may be of use in the treatment of heart disease. Among metabolically relevant tissues, this gene also has moderate expression in pancreas (CT=33), thyroid (CT=31), pituitary gland (CT=30), skeletal muscle (CT=29) and fetal and adult liver (CTs=28-29).

The NOV5 gene is expressed at high levels throughout the CNS, including in amygdala, hippocampus, cerebellum, thalamus, cerebral cortex and spinal cord. Tetraspanins are expressed in response to CNS injuries such as stroke and spinal cord/head trauma, possibly playing a role in reactive gliosis. Reactive gliosis is also a hallmark of neurodegeneration in diseases such as Alzheimer's, Parkinson's, Huntington's, spinocerebellar ataxia and progressive supranuclear palsy. Therefore, therapeutic modulation of the NOV5 gene or its protein product may be beneficial in the treatment or prevention of these conditions/diseases.

Panel 1.3D Summary: Ag610 Expression of the NOV5 gene is associated primarily with normal tissue samples rather than the samples derived from the cultured cell lines on this panel, consistent with what was observed on Panels 1.1 and 1.2. Strikingly, this gene appears to be under expressed in ovarian, breast and lung cancer cell lines relative to normal controls. These observations suggest that the NOV5 gene product may have utility as a protein therapeutic in the treatment of ovarian, breast and lung cancers.

The NOV5 gene is also moderately expressed in many regions of the brain, including the amygdala, thalamus, hippocampus, and cerebral cortex, with highest expression in the cerebellum (CT value=28.6). Expression is also detected in the spinal cord. The gene encoded by the NOV5 gene encodes a putative tetraspanin. Tetraspanins are involved in neuron to astrocyte signalling (Kelic et al., CD81 regulates neuron-induced astrocyte cell-cycle exit. Mol. Cell. Neurosci. 17: 551-560, 2001; Brenz Verca et al., Cocaine-induced expression of the tetraspanin CD81 and its relation to hypothalamic function. Mol. Cell Neurosci. 17: 303-316, 2001). Astrocytes are of interest in neuronal regeneration as they form glial scars in response to CNS injury (i.e., spinal cord injury, brain trauma, etc). Glial scars form a physical barrier to growing axons and dendrites, limiting the amount of CNS repair possible. Astrocytes are also critical to the process of compensatory synaptogenesis in that they are integral in the brain's cholesterol transport system and are involved in the transport of hydrophobic membrane/synapse components to neurons. The selective modulations and/or activation of this protein could therefore be of therapeutic value in the treatment of CNS injury (stroke, head trauma, spinal cord injury) or neurodegeneration (Alzheimer's, Parkinson's, Huntington's, spinocerebellar ataxia, etc).

In addition, low expression of this gene is detected in pancreas, liver and adipose with moderate expression in adrenal gland, thyroid, pituitary gland, heart, and skeletal muscle. Expression in the metabolic tissues skeletal muscle, liver and pancreas suggest that the NOV5 gene may be involved in metabolic control processes and serve as a drug target for metabolic diseases, including obesity and diabetes. In addition, this gene may play a role in normal neuroendocrine function and disregulation may lead to diseases of the endocrine system.

Panel 2D Summary: Ag610 The NOV5 gene is widely expressed among the samples in panel 2D with the highest expression occurring in a kidney cancer sample (CT value=28.2). Of specific interest is the differential over expression of the NOV5 gene in 4/9 kidney cancers and 4/4 gastric cancers relative to the adjacent normal tissue controls. In addition, there is also slight under expression in 2/2 prostate cancers and 5/6 colon cancers relative to the adjacent normal tissue controls. Thus, therapeutic modulation of the expression of the NOV5 gene could have beneficial consequences to the treatment of several types of cancers.

Panel 4D Summary: Ag610/Ag1199/Ag2940 The NOV5 transcript is expressed in normal organs, untreated endothelial cells and polarized resting T cells. Furthermore, expression is reduced in endothelial cells treated with IL-1 and TNF alpha. The expression pattern is consistent in three experiments using different probe/primer sets. Protein therapeutics designed with the protein encoded for by this transcript could interact with the cognate ligand for the NOV5 protein to reduce or inhibit inflammation due to the exposure of endothelium to the pro-inflammatory cytokines. Alternatively, since many tetraspanins are involved as part of a receptor complexes, the putative tetraspanin encoded by the NOV5 gene may actually function in the initial steps of activation and, therefore, an antibody against the protein encoded for by this transcript may block subsequent steps of endothelial cell activation. Both of these therapeutics may be important in the treatment of diseases such as asthma, emphysema, arthritis, allergy, psoriasis and IBD.

Panel CNSD.01 Summary: Ag610 The NOV5 gene is expressed at low to undetectable levels (CT values>34.5) in all of the samples on this panel; however, the brain tissues in which highest expression was observed in Panels 1.1, 1.2 and 1.3D are not represented here.

NOV7: Butyrophilin-Like Receptor

Expression of gene NOV7 (AC016572_da1) was assessed using the primer-probe set Ag2030, described in Table X. Results of the RTQ-PCR runs are shown in Tables Y and Z.

TABLE X
Probe Name Ag2030

| | | | | Start | SEQ ID |
|---|---|---|---|---|---|
| Primers | Sequences | TM | Length | Position | NO: |
| Forward | 5′-CTGCGTTTCTGATCTGAAAACT-3′ | 58.7 | 22 | 900 | 107 |
| Probe | TET-5′-ACCCATAGAAAAGCTCCCCAGGAGGT-3′-TAMRA | 69.6 | 26 | 925 | 108 |
| Reverse | 5′-CCACCACACTCTTCCTTGTAAA-3′ | 59.1 | 22 | 970 | 109 |

TABLE Y
Panel 1.3D

| | Relative | | Relative |
|---|---|---|---|
| | Expression (%) | | Expression (%) |
| | 1.3dx4tm5433t | | 1.3dx4tm5433t |
| Tissue Name | _ag2030_a2 | Tissue Name | _ag2030_a2 |
| Liver adenocarcinoma | 0.0 | Kidney (fetal) | 6.5 |
| Pancreas | 3.3 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 3.8 | Renal ca. ACHN | 0.0 |
| Salivary gland | 0.3 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 1.6 |
| Brain (amygdala) | 0.3 | Liver ca. (hepatoblast) HepG2 | 0.0 |
| Brain (cerebellum) | 0.0 | Lung | 28.0 |
| Brain (hippocampus) | 0.0 | Lung (fetal) | 12.4 |
| Brain (substantia nigra) | 0.0 | Lung ca. (small cell) LX-1 | 0.4 |
| Brain (thalamus) | 0.3 | Lung ca. (small cell) NCI-H69 | 0.0 |
| Cerebral Cortex | 0.0 | Lung ca. (s.cell var.) SHP-77 | 0.0 |
| Spinal cord | 3.2 | Lung ca. (large cell) NCI-H460 | 7.6 |
| CNS ca. (glio/astro) U87-MG | 0.0 | Lung ca. (non-sm.cell) A549 | 1.1 |
| CNS ca. (glio/astro) U-118-MG | 0.0 | Lung ca. (non-s.cell) NCI-H23 | 0.0 |
| CNS ca. (astro) SW1783 | 0.0 | Lung ca. (non-s.cell) HOP-62 | 0.0 |
| CNS ca.* (neuro; met) SK-N- | 0.0 | Lung ca. (non-s.cl) NCI-H522 | 0.0 |
| AS | | | |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 0.0 |
| CNS ca. (glio) U251 | 0.0 | Breast ca.* (pl. effusion) MCF-7 | 0.0 |
| CNS ca. (glio) SF-295 | 0.9 | Breast ca.* (pl.ef) MDA-MB- | 0.0 |
| | | 231 | |
| Heart (fetal) | 0.0 | Breast ca.* (pl. effusion) T47D | 0.0 |
| Heart | 0.0 | Breast ca. BT-549 | 0.0 |
| Fetal Skeletal | 0.0 | Breast ca. MDA-N | 0.0 |
| Skeletal muscle | 0.0 | Ovary | 0.0 |
| Bone marrow | 30.6 | Ovarian ca. OVCAR-3 | 0.0 |
| Thymus | 0.0 | Ovarian ca. OVCAR-4 | 0.0 |
| Spleen | 3.0 | Ovarian ca. OVCAR-5 | 1.0 |
| Lymph node | 4.3 | Ovarian ca. OVCAR-8 | 0.0 |
| Colorectal | 73.7 | Ovarian ca. IGROV-1 | 0.0 |
| Stomach | 15.7 | Ovarian ca.* (ascites) SK-OV-3 | 0.0 |
| Small intestine | 100.0 | Uterus | 0.8 |
| Colon ca. SW480 | 0.0 | Placenta | 0.0 |
| Colon ca.* (SW480 met) SW620 | 0.0 | Prostate | 0.0 |
| Colon ca. HT29 | 0.0 | Prostate ca.* (bone met) PC-3 | 0.0 |
| Colon ca. HCT-116 | 0.0 | Testis | 15.7 |
| Colon ca. CaCo-2 | 40.1 | Melanoma Hs688(A).T | 0.0 |
| 83219 CC Well to Mod Diff | 1.8 | Melanoma* (met) Hs688(B).T | 0.0 |
| (ODO3866) | | | |
| Colon ca. HCC-2998 | 8.1 | Melanoma UACC-62 | 0.0 |
| Gastric ca.* (liver met) NCI- | 0.0 | Melanoma M14 | 0.0 |
| N87 | | | |
| Bladder | 10.6 | Melanoma LOX IMVI | 0.0 |
| Trachea | 1.6 | Melanoma* (met) SK-MEL-5 | 0.0 |
| Kidney | 0.0 | Adipose | 1.8 |

TABLE Z
Panel 4D

| | Relative | | Relative |
|---|---|---|---|
| | Expression (%) | | Expression (%) |
| | 4dx4tm4450t_{—} | | 4dx4tm4450t_{—} |
| Tissue Name | ag2030_b1 | Tissue Name | ag2030_b1 |
| 93768_Secondary Th1_anti- | 0.1 | 93100_HUVEC | 0.0 |
| CD28/anti-CD3 | | (Endothelial)_IL-1b | |
| 93769_Secondary Th2_anti- | 0.0 | 93779_HUVEC | 0.0 |
| CD28/anti-CD3 | | (Endothelial)_IFN gamma | |
| 93770_Secondary Tr1_anti- | 0.0 | 93102_HUVEC | 0.1 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 0.0 |
| 4-6 in IL-2 | | epithelium_TNFa (4 ng/ml) and | |
| | | IL1b (1 ng/ml)** | |
| 93566_primary Th2_resting dy | 0.1 | 93347_Small Airway | 0.0 |
| 4-6 in IL-2 | | Epithelium_none | |
| 93567_primary Tr1_resting dy | 0.0 | 93348_Small Airway | 0.0 |
| 4-6 in IL-2 | | Epithelium_TNFa (4 ng/ml) | |
| | | and IL1b (1 ng/ml) | |
| 93351_CD45RA CD4 | 0.0 | 92668_Coronery Artery | 0.0 |
| lymphocyte_anti-CD28/anti- | | SMC_resting | |
| CD3 | | | |
| 93352_CD45RO CD4 | 0.0 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.1 | 93107_astrocytes_resting | 0.0 |
| CD28/anti-CD3 | | | |
| 93353_chronic CD8 | 0.0 | 93108_astrocytes_TNFa (4 | 0.0 |
| Lymphocytes 2ry_resting dy 4-6 | | ng/ml) and IL1b (1 ng/ml) | |
| in IL-2 | | | |
| 93574_chronic CD8 | 0.2 | 92666_KU-812 | 0.0 |
| Lymphocytes 2ry_activated | | (Basophil)_resting | |
| CD3/CD28 | | | |
| 93354_CD4_none | 0.0 | 92667_KU-812 | 0.0 |
| | | (Basophil)_PMA/ionoycin | |
| 93252_Secondary | 0.1 | 93579_CCD1106 | 0.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa and | |
| | | IFNg** | |
| 93788_LAK cells_IL-2 | 0.0 | 93791_Liver Cirrhosis | 0.6 |
| 93787_LAK cells_IL-2 + IL-12 | 0.0 | 93792_Lupus Kidney | 0.0 |
| 93789_LAK cells_IL-2 + IFN | 0.0 | 93577_NCI-H292 | 0.0 |
| gamma | | | |
| 93790_LAK cells_IL-2 + IL-18 | 0.0 | 93358_NCI-H292_IL-4 | 0.0 |
| 93104_LAK | 0.0 | 93360_NCI-H292_IL-9 | 0.0 |
| cells_PMA/ionomycin and IL- | | | |
| 18 | | | |
| 93578_NK Cells IL-2_resting | 0.0 | 93359_NCI-H292_IL-13 | 0.0 |
| 93109_Mixed Lymphocyte | 0.0 | 93357_NCI-H292_IFN gamma | 0.0 |
| Reaction_Two Way MLR | | | |
| 93110_Mixed Lymphocyte | 0.0 | 93777_HPAEC- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 0.0 |
| (PBMCs)_resting | | Fibroblast_none | |
| 93113_Mononuclear Cells | 0.3 | 93253_Normal Human Lung | 0.0 |
| (PBMCs)_PWM | | Fibroblast_TNFa (4 ng/ml) and | |
| | | IL-1b (1 ng/ml) | |
| 93114_Mononuclear Cells | 0.0 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B | 0.0 | 93255_Normal Human Lung | 0.0 |
| cell)_ionomycin | | Fibroblast_IL-13 | |
| 93349_B lymphocytes_PWM | 2.5 | 93258_Normal Human Lung | 0.0 |
| | | Fibroblast_IFN gamma | |
| 93350_B lymphoytes_CD40L | 0.0 | 93106_Dermal Fibroblasts | 0.0 |
| and IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/PMAionomycin | | CCD1070_IL-1 beta 1 ng/ml | |
| 93356_Dendritic Cells_none | 0.0 | 93772_dermal fibroblast_IFN | 0.0 |
| | | gamma | |
| 93355_Dendritic Cells_LPS | 0.0 | 93771_dermal fibroblast_IL-4 | 0.0 |
| 100 ng/ml | | | |
| 93775_Dendritic Cells_anti- | 0.0 | 93259_IBD Colitis 1** | 0.0 |
| CD40 | | | |
| 93774_Monocytes_resting | 0.0 | 93260_IBD Colitis 2 | 0.0 |
| 93776_Monocytes_LPS 50 ng/ml | 0.0 | 93261_IBD Crohns | 5.3 |
| 93581_Macrophages_resting | 0.0 | 735010_Colon_normal | 100.0 |
| 93582_Macrophages_LPS 100 ng/ml | 0.0 | 735019_Lung_none | 0.5 |
| 93098_HUVEC | 0.0 | 64028-1_Thymus_none | 0.5 |
| (Endothelial)_none | | | |
| 93099_HUVEC | 0.0 | 64030-1_Kidney_none | 0.0 |
| (Endothelial)_starved | | | |

Panel 1.3D Summary: Ag2030 The NOV7 gene appears to be most prominently expressed in gastrointestinal tissues. Expression of this gene is highest in colon and small intestine (CT=31) and it is also significantly expressed in stomach tissue. Thus, the expression of the NOV7 gene might be useful as a marker of gastrointestinal tissue, and specifically of small intestine, stomach or colorectal tissue.

Panel 4D Summary: Ag2030 NOV7 gene expression on this panel is limited essentially to normal colon. These results are consistent with what is observed in Panel 10.3D. Interestingly, this gene is not expressed or is expressed at lower levels in IBD colitis and Crohn's disease. Therefore, this gene could be used to distinguish normal colon from diseased colon. The NOV7 gene encodes a protein with homology to the butyrophilin-like receptor. The butyrophilin-like membrane proteins are similar to the B7 family of co-stimulatory factors. B7 proteins are expressed on many cell types and function in the process of antigen presentation to T lymphocytes in the stimulation of the immune response. Recently identified B7 family members have included inhibitory proteins that reduced the activation of T lymphocytes upon cell-cell interaction. Thus, the NOV7 gene product may modulate the functions associated with antigen presentation to T cells. Therefore, replacement of the NOV7 gene product by gene therapy or using the isolated extracellular domain of the NOV7 protein may function as useful therapeutics in the treatment of IBD colitis and Crohn's disease.

NOV8: MEGF/Fibrillin-Like

Expression of gene NOV8 (101360122_EXT4) was assessed using the primer-probe sets Ag192 as well as Ag1391 and Ag2890 (identical sequences), described in Tables AA and AB. Results of the RTQ-PCR runs are shown in Tables AC, AD, AE, AF, AG, and AH.

TABLE AA
Probe Name Ag192

| | | | | Start | SEQ ID |
|---|---|---|---|---|---|
| Primers | Sequences | TM | Length | Position | NO: |
| Forward | 5′-TGTGCCGAGGGCAACG-3′ | | 16 | 208 | 110 |
| Probe | FAM-5′-TAGCTGCCCATCATGTTGACACAGCTCT-3′-TAMRA | | 28 | 236 | 111 |
| Reverse | 5′-AGAAGCCTTCCCGGCAGT-3′ | | 18 | 272 | 112 |

TABLE AB
Probe Name Ag1391/Ag2890

| | | | | Start | SEQ ID |
|---|---|---|---|---|---|
| Primers | Sequences | TM | Length | Position | NO: |
| Forward | 5′-CTTATGAGACCTGCCAGACCTA-3′ | 58.5 | 22 | 2660 | 113 |
| Probe | FAM-5′-CTTCACTGCCCGTTCCAGGAAGCT-3′-TAMRA | 70.7 | 24 | 2697 | 114 |
| Reverse | 5′-CTCGCTTGTCTTGAAGTTGATC-3′ | 59.1 | 22 | 2724 | 115 |

TABLE AC
Panel 1 (Ag192)

Relative
Expression (%)

| Tissue Name | tm383f | tm356f |
|---|---|---|
| Endothelial cells | 32.1 | 0.0 |
| Endothelial cells (treated) | 15.4 | 0.0 |
| Pancreas | 19.5 | 0.2 |
| Pancreatic ca. CAPAN 2 | 94.0 | 0.0 |
| Adipose | 23.3 | 0.3 |
| Adrenal gland | 38.2 | 4.0 |
| Thyroid | 52.8 | 28.9 |
| Salivary gland | 15.9 | 1.5 |
| Pituitary gland | 16.8 | 1.2 |
| Brain (fetal) | 37.4 | 1.2 |
| Brain (whole) | 23.2 | 0.7 |
| Brain (amygdala) | 33.0 | 0.7 |
| Brain (cerebellum) | 18.9 | 12.9 |
| Brain (hippocampus) | 23.0 | 0.3 |
| Brain (substantia nigra) | 17.8 | 0.5 |
| Brain (thalamus) | 26.8 | 1.0 |
| Brain (hypothalamus) | 42.9 | 8.1 |
| Spinal cord | 8.8 | 0.7 |
| CNS ca. (glio/astro) U87-MG | 18.4 | 0.0 |
| CNS ca. (glio/astro) U-118-MG | 15.3 | 2.6 |
| CNS ca. (astro) SW1783 | 6.9 | 0.0 |
| CNS ca.* (neuro; met) SK-N-AS | 18.4 | 1.2 |
| CNS ca. (astro) SF-539 | 10.9 | 0.9 |
| CNS ca. (astro) SNB-75 | 100.0 | 100.0 |
| CNS ca. (glio) SNB-19 | 15.3 | 6.5 |
| CNS ca. (glio) U251 | 31.4 | 5.0 |
| CNS ca. (glio) SF-295 | 11.2 | 1.9 |
| Heart | 22.8 | 3.1 |
| Skeletal muscle | 18.7 | 0.0 |
| Bone marrow | 16.5 | 0.0 |
| Thymus | 2.0 | 0.9 |
| Spleen | 14.4 | 0.2 |
| Lymph node | 20.2 | 0.6 |
| Colon (ascending) | 9.6 | 0.0 |
| Stomach | 2.2 | 0.2 |
| Small intestine | 16.8 | 0.0 |
| Colon ca. SW480 | 7.7 | 0.0 |
| Colon ca.* (SW480 met) SW620 | 12.7 | 0.2 |
| Colon ca. HT29 | 8.7 | 0.0 |
| Colon ca. HCT-116 | 14.6 | 0.2 |
| Colon ca. CaCo-2 | 9.3 | 0.7 |
| Colon ca. HCT-15 | 7.5 | 0.2 |
| Colon ca. HCC-2998 | 9.7 | 0.2 |
| Gastric ca.* (liver met) NCI-N87 | 5.7 | 0.0 |
| Bladder | 43.5 | 8.7 |
| Trachea | 15.5 | 2.0 |
| Kidney | 21.5 | 3.1 |
| Kidney (fetal) | 52.1 | 34.2 |
| Renal ca. 786-0 | 6.9 | 32.8 |
| Renal ca. A498 | 7.9 | 0.0 |
| Renal ca. RXF 393 | 18.2 | 0.4 |
| Renal ca. ACHN | 4.8 | 0.0 |
| Renal ca. UO-31 | 10.7 | 0.1 |
| Renal ca. TK-10 | 9.5 | 1.7 |
| Liver | 21.3 | 0.0 |
| Liver (fetal) | 17.4 | 0.0 |
| Liver ca. (hepatoblast) HepG2 | 66.9 | 0.3 |
| Lung | 2.9 | 1.0 |
| Lung (fetal) | 17.3 | 1.1 |
| Lung ca. (small cell) LX-1 | 8.8 | 0.0 |
| Lung ca. (small cell) NCI-H69 | 10.7 | 0.0 |
| Lung ca. (s.cell var.) SHP-77 | 33.4 | 3.7 |
| Lung ca. (large cell) NCI-H460 | 13.0 | 0.5 |
| Lung ca. (non-sm. cell) A549 | 9.7 | 1.0 |
| Lung ca. (non-s.cell) NCI-H23 | 18.4 | 4.4 |
| Lung ca. (non-s.cell) HOP-62 | 8.1 | 1.5 |
| Lung ca. (non-s.cl) NCI-H522 | 38.7 | 21.5 |
| Lung ca. (squam.) SW 900 | 8.3 | 1.0 |
| Lung ca. (squam.) NCI-H596 | 27.0 | 4.4 |
| Mammary gland | 23.3 | 4.1 |
| Breast ca.* (pl. effusion) MCF-7 | 9.5 | 0.0 |
| Breast ca.* (pl.ef) MDA-MB-231 | 21.2 | 0.5 |
| Breast ca.* (pl. effusion) T47D | 11.9 | 0.4 |
| Breast ca. BT-549 | 16.8 | 0.5 |
| Breast ca. MDA-N | 12.9 | 0.0 |
| Ovary | 2.1 | 0.0 |
| Ovarian ca. OVCAR-3 | 14.4 | 0.5 |
| Ovarian ca. OVCAR-4 | 12.9 | 5.6 |
| Ovarian ca. OVCAR-5 | 8.4 | 2.1 |
| Ovarian ca. OVCAR-8 | 11.3 | 7.2 |
| Ovarian ca. IGROV-1 | 8.8 | 0.0 |
| Ovarian ca.* (ascites) SK-OV-3 | 21.3 | 1.2 |
| Uterus | 22.5 | 4.8 |
| Placenta | 48.0 | 0.2 |
| Prostate | 18.7 | 2.7 |
| Prostate ca.* (bone met) PC-3 | 27.4 | 24.1 |
| Testis | 3.1 | 17.8 |
| Melanoma Hs688(A).T | 25.0 | 0.0 |
| Melanoma* (met) Hs688(B).T | 69.3 | 51.4 |
| Melanoma UACC-62 | 17.9 | 0.0 |
| Melanoma M14 | 6.9 | 0.0 |
| Melanoma LOX IMVI | 5.1 | 0.3 |
| Melanoma* (met) SK-MEL-5 | 17.6 | 0.4 |
| Melanoma SK-MEL-28 | 20.7 | 0.9 |

TABLE AD
Panel 1.1

| | Relative | | Relative |
|---|---|---|---|
| | Expression (%) | | Expression (%) |
| Tissue Name | 1.1tm720f_ag192 | Tissue Name | 1.1tm720f_ag192 |
| Adipose | 0.5 | Renal ca. TK-10 | 7.1 |
| Adrenal gland | 7.0 | Renal ca. UO-31 | 1.6 |
| Bladder | 8.6 | Renal ca. RXF 393 | 0.7 |
| Brain (amygdala) | 0.0 | Liver | 1.2 |
| Brain (cerebellum) | 4.7 | Liver (fetal) | 0.0 |
| Brain (hippocampus) | 1.6 | Liver ca. (hepatoblast) | 1.4 |
| | | HepG2 | |
| Brain (substantia nigra) | 13.1 | Lung | 0.2 |
| Brain (thalamus) | 3.8 | Lung (fetal) | 3.3 |
| Cerebral Cortex | 2.1 | Lung ca (non-s.cell) HOP-62 | 49.3 |
| Brain (fetal) | 2.4 | Lung ca. (large cell) NCI- | 5.8 |
| | | H460 | |
| Brain (whole) | 2.4 | Lung ca. (non-s.cell) NCI- | 12.2 |
| | | H23 | |
| CNS ca. (glio/astro) U-118- | 7.1 | Lung ca. (non-s.cl) NCI- | 77.9 |
| MG | | H522 | |
| CNS ca. (astro) SF-539 | 0.0 | Lung ca. (non-sm. cell) A549 | 5.8 |
| CNS ca. (astro) SNB-75 | 78.5 | Lung ca. (s.cell var.) SHP-77 | 0.8 |
| CNS ca. (astro) SW1783 | 0.2 | Lung ca. (small cell) LX-1 | 2.5 |
| CNS ca. (glio) U251 | 19.6 | Lung ca. (small cell) NCI- | 5.3 |
| | | H69 | |
| CNS ca. (glio) SF-295 | 9.2 | Lung ca. (squam.) SW 900 | 3.8 |
| CNS ca. (glio) SNB-19 | 15.9 | Lung ca. (squam.) NCI-H596 | 14.4 |
| CNS ca. (glio/astro) U87-MG | 1.9 | Lymph node | 1.9 |
| CNS ca.* (neuro; met) SK- | 7.0 | Spleen | 0.0 |
| N-AS | | | |
| Mammary gland | 0.3 | Thymus | 0.3 |
| Breast ca. BT-549 | 1.5 | Ovary | 5.6 |
| Breast ca. MDA-N | 1.6 | Ovarian ca. IGROV-1 | 3.1 |
| Breast ca.* (pl. effusion) | 5.3 | Ovarian ca. OVCAR-3 | 8.6 |
| T47D | | | |
| Breast ca.* (pl. effusion) | 0.9 | Ovarian ca. OVCAR-4 | 27.2 |
| MCF-7 | | | |
| Breast ca.* (pl.ef) MDA-MB- | 5.5 | Ovarian ca. OVCAR-5 | 9.9 |
| 231 | | | |
| Small intestine | 2.2 | Ovarian ca. OVCAR-8 | 17.6 |
| Colorectal | 0.9 | Ovarian ca.* (ascites) SK- | 11.3 |
| | | OV-3 | |
| Colon ca. HT29 | 0.3 | Pancreas | 10.0 |
| Colon ca. CaCo-2 | 0.0 | Pancreatic ca. CAPAN 2 | 0.0 |
| Colon ca. HCT-15 | 2.9 | Pituitary gland | 10.9 |
| Colon ca. HCT-116 | 1.5 | Placenta | 1.6 |
| Colon ca. HCC-2998 | 3.8 | Prostate | 2.6 |
| Colon ca. SW480 | 0.3 | Prostate ca.* (bone met) PC-3 | 80.7 |
| Colon ca.* (SW480 | 3.1 | Salivary gland | 8.1 |
| met) SW620 | | | |
| Stomach | 5.6 | Trachea | 3.9 |
| Gastric ca.* (liver met) NCI- | 1.8 | Spinal cord | 5.8 |
| N87 | | | |
| Heart | 39.2 | Testis | 3.3 |
| Fetal Skeletal | 1.0 | Thyroid | 100.0 |
| Skeletal muscle | 7.4 | Uterus | 2.1 |
| Endothelial cells | 7.9 | Melanoma M14 | 1.4 |
| Heart (fetal) | 12.4 | Melanoma LOX IMVI | 0.5 |
| Kidney | 27.4 | Melanoma UACC-62 | 0.2 |
| Kidney (fetal) | 37.1 | Melanoma SK-MEL-28 | 3.5 |
| Renal ca. 786-0 | 2.0 | Melanoma* (met) SK-MEL-5 | 1.2 |
| Renal ca. A498 | 0.6 | Melanoma Hs688(A).T | 0.3 |
| Renal ca. ACHN | 2.7 | Melanoma* (met) | 66.0 |
| | | Hs688(B).T | |

TABLE AE
Panel 1.2

| | Relative Expression(%) | |
|---|---|---|
| | 1.2tm- | 1.2tm- |
| | 1632f_{—} | 1669f_{—} |
| Tissue Name | ag1391 | ag1391* |
| Endothelial cells | 6.2 | 4.2 |
| Heart (fetal) | 6.0 | 9.2 |
| Pancreas | 0.7 | 0.4 |
| Pancreatic ca. CAPAN 2 | 0.1 | 0.6 |
| Adrenal Gland (new lot*) | 27.0 | 17.4 |
| Thyroid | 15.0 | 4.9 |
| Salivary gland | 6.8 | 7.4 |
| Pituitary gland | 0.0 | 1.1 |
| Brain (fetal) | 4.3 | 0.9 |
| Brain (whole) | 4.3 | 1.5 |
| Brain (amygdala) | 1.4 | 1.6 |
| Brain (cerebellum) | 3.5 | 1.1 |
| Brain (hippocampus) | 1.4 | 3.8 |
| Brain (thalamus) | 1.2 | 3.7 |
| Cerebral Cortex | 2.4 | 8.0 |
| Spinal cord | 0.5 | 0.5 |
| CNS ca. (glio/astro) U87-MG | 16.6 | 2.5 |
| CNS ca. (glio/astro) U-118-MG | 54.0 | 7.0 |
| CNS ca. (astro) SW1783 | 4.5 | 1.3 |
| CNS ca.* (neuro; met) SK-N-AS | 21.3 | 3.9 |
| CNS ca. (astro) SF-539 | 6.5 | 3.1 |
| CNS ca. (astro) SNB-75 | 100.0 | 49.3 |
| CNS ca. (glio) SNB-19 | 14.8 | 9.1 |
| CNS ca. (glio) U251 | 0.0 | 10.9 |
| CNS ca. (glio) SF-295 | 1.4 | 8.9 |
| Heart | 33.2 | 69.3 |
| Skeletal Muscle (new lot*) | 1.1 | 5.3 |
| Bone marrow | 0.3 | 0.6 |
| Thymus | 0.3 | 1.1 |
| Spleen | 0.4 | 1.0 |
| Lymph node | 1.8 | 0.3 |
| Colorectal | 0.6 | 1.6 |
| Stomach | 14.1 | 1.9 |
| Small intestine | 2.9 | 3.8 |
| Colon ca. SW480 | 0.3 | 1.3 |
| Colon ca.* (SW480 met) SW620 | 5.6 | 2.9 |
| Colon ca. HT29 | 0.4 | 0.8 |
| Colon ca. HCT-116 | 3.9 | 5.9 |
| Colon ca. CaCo-2 | 1.8 | 5.2 |
| 83219 CC Well to Mod Diff (ODO3866) | 9.2 | 3.6 |
| Colon ca. HCC-2998 | 3.3 | 7.1 |
| Gastric ca.* (liver met) NCI-N87 | 5.8 | 2.1 |
| Bladder | 4.9 | 8.6 |
| Trachea | 1.6 | 0.4 |
| Kidney | 22.4 | 30.6 |
| Kidney (fetal) | 4.4 | 15.2 |
| Renal ca. 786-0 | 1.2 | 1.4 |
| Renal ca. A498 | 0.7 | 1.1 |
| Renal ca. RXF 393 | 1.7 | 2.1 |
| Renal ca. ACHN | 1.8 | 4.7 |
| Renal ca. UO-31 | 0.5 | 3.1 |
| Renal ca. TK-10 | 1.6 | 6.0 |
| Liver | 0.8 | 2.1 |
| Liver (fetal) | 0.4 | 0.9 |
| Liver ca. (hepatoblast) HepG2 | 2.0 | 5.3 |
| Lung | 0.2 | 0.3 |
| Lung (fetal) | 0.3 | 0.4 |
| Lung ca. (small cell) LX-1 | 2.0 | 4.0 |
| Lung ca. (small cell) NCI-H69 | 10.4 | 5.8 |
| Lung ca. (s.cell var.) SHP-77 | 0.3 | 1.7 |
| Lung ca. (large cell) NCI-H460 | 4.1 | 6.9 |
| Lung ca. (non-sm. cell) A549 | 2.5 | 6.5 |
| Lung ca. (non-s.cell) NCI-H23 | 5.6 | 14.7 |
| Lung ca. (non-s.cell) HOP-62 | 3.9 | 20.2 |
| Lung ca. (non-s.cl) NCI-H522 | 23.8 | 100.0 |
| Lung ca. (squam.) SW 900 | 2.9 | 5.8 |
| Lung ca. (squam.) NCI-H596 | 7.1 | 15.8 |
| Mammary gland | 6.2 | 1.7 |
| Breast ca.* (pl. effusion) MCF-7 | 3.4 | 1.8 |
| Breast ca.* (pl.ef) MDA-MB-231 | 4.2 | 3.2 |
| Breast ca.* (pl. effusion) T47D | 15.2 | 4.3 |
| Breast ca. BT-549 | 8.5 | 2.1 |
| Breast ca. MDA-N | 15.9 | 4.9 |
| Ovary | 3.0 | 4.5 |
| Ovarian ca. OVCAR-3 | 7.9 | 5.9 |
| Ovarian ca. OVCAR-4 | 5.8 | 12.9 |
| Ovarian ca. OVCAR-5 | 8.0 | 8.7 |
| Ovarian ca. OVCAR-8 | 18.8 | 16.5 |
| Ovarian ca. IGROV-1 | 2.1 | 2.4 |
| Ovarian ca.* (ascites) SK-OV-3 | 29.7 | 8.7 |
| Uterus | 3.8 | 2.0 |
| Placenta | 0.6 | 0.7 |
| Prostate | 3.5 | 7.8 |
| Prostate ca.* (bone met) PC-3 | 30.8 | 61.1 |
| Testis | 3.4 | 0.6 |
| Melanoma Hs688(A).T | 0.6 | 1.6 |
| Melanoma* (met) Hs688(B).T | 28.1 | 59.0 |
| Melanoma UACC-62 | 0.9 | 2.1 |
| Melanoma M14 | 1.2 | 4.4 |
| Melanoma LOX IMVI | 0.5 | 1.8 |
| Melanoma* (met) SK-MEL-5 | 1.2 | 3.4 |
| Adipose | 7.1 | 14.8 |

TABLE AF
Panel 1.3D

| | Relative | | Relative |
|---|---|---|---|
| | Expression(%) | | Expression(%) |
| | 1.3dx4tm_5798f_{—} | | 1.3dx4tm_5798f_{—} |
| Tissue Name | ag2890_a1 | Tissue Name | ag2890_a1 |
| Liver adenocarcinoma | 2.3 | Kidney (fetal) | 22.0 |
| Pancreas | 0.5 | Renal ca. 786-0 | 1.9 |
| Pancreatic ca. CAPAN 2 | 0.2 | Renal ca. A498 | 7.7 |
| Adrenal gland | 1.7 | Renal ca. RXF 393 | 3.3 |
| Thyroid | 15.7 | Renal ca. ACHN | 1.7 |
| Salivary gland | 0.6 | Renal ca. UO-31 | 0.3 |
| Pituitary gland | 1.5 | Renal ca. TK-10 | 2.0 |
| Brain (fetal) | 4.8 | Liver | 0.3 |
| Brain (whole) | 1.9 | Liver (fetal) | 0.3 |
| Brain (amygdala) | 1.0 | Liver ca. (hepatoblast) HepG2 | 0.9 |
| Brain (cerebellum) | 3.6 | Lung | 0.7 |
| Brain (hippocampus) | 1.4 | Lung (fetal) | 1.4 |
| Brain (substantia nigra) | 2.1 | Lung ca. (small cell) LX-1 | 1.5 |
| Brain (thalamus) | 1.8 | Lung ca. (small cell) NCI-H69 | 2.2 |
| Cerebral Cortex | 1.8 | Lung ca. (s.cell var.) SHP-77 | 7.3 |
| Spinal cord | 1.9 | Lung ca. (large cell) NCI-H460 | 0.8 |
| CNS ca. (glio/astro) U87-MG | 1.3 | Lung ca. (non-sm. cell) A549 | 4.0 |
| CNS ca. (glio/astro) U-118-MG | 4.1 | Lung ca. (non-s.cell) NCI-H23 | 4.8 |
| CNS ca. (astro) SW1783 | 2.0 | Lung ca. (non-s.cell) HOP-62 | 3.6 |
| CNS ca.* (neuro; met) SK-N- | 1.8 | Lung ca. (non-s.cl) NCI-H522 | 8.1 |
| AS | | | |
| CNS ca. (astro) SF-539 | 1.9 | Lung ca. (squam.) SW 900 | 1.7 |
| CNS ca. (astro) SNB-75 | 28.6 | Lung ca. (squam.) NCI-H596 | 8.0 |
| CNS ca. (glio) SNB-19 | 3.9 | Mammary gland | 0.8 |
| CNS ca. (glio) U251 | 20.2 | Breast ca.* (pl. effusion) MCF-7 | 1.1 |
| CNS ca. (glio) SF-295 | 2.3 | Breast ca.* (pl.ef) MDA-MB- | 2.9 |
| | | 231 | |
| Heart (fetal) | 3.5 | Breast ca.* (pl. effusion) T47D | 2.7 |
| Heart | 3.9 | Breast ca. BT-549 | 1.0 |
| Fetal Skeletal | 1.8 | Breast ca. MDA-N | 0.6 |
| Skeletal muscle | 0.7 | Ovary | 1.7 |
| Bone marrow | 0.4 | Ovarian ca. OVCAR-3 | 2.4 |
| Thymus | 1.2 | Ovarian ca. OVCAR-4 | 3.6 |
| Spleen | 0.1 | Ovarian ca. OVCAR-5 | 3.7 |
| Lymph node | 0.8 | Ovarian ca. OVCAR-8 | 1.7 |
| Colorectal | 3.6 | Ovarian ca. IGROV-1 | 0.2 |
| Stomach | 0.5 | Ovarian ca.* (ascites) SK-OV-3 | 7.5 |
| Small intestine | 0.4 | Uterus | 0.8 |
| Colon ca. SW480 | 0.8 | Placenta | 0.2 |
| Colon ca.* (SW480 met)SW620 | 4.7 | Prostate | 0.7 |
| Colon ca. HT29 | 0.4 | Prostate ca.* (bone met) PC-3 | 12.2 |
| Colon ca. HCT-116 | 0.2 | Testis | 1.4 |
| Colon ca. CaCo-2 | 1.7 | Melanoma Hs688(A).T | 9.3 |
| 83219 CC Well to Mod Diff | 7.0 | Melanoma* (met) Hs688(B).T | 100.0 |
| (ODO3866) | | | |
| Colon ca. HCC-2998 | 1.1 | Melanoma UACC-62 | 0.8 |
| Gastric ca.* (liver met) NCI- | 1.3 | Melanoma M14 | 0.5 |
| N87 | | | |
| Bladder | 1.5 | Melanoma LOX IMVI | 1.9 |
| Trachea | 0.2 | Melanoma* (met) SK-MEL-5 | 0.3 |
| Kidney | 3.8 | Adipose | 0.9 |

TABLE AG
Panel 2D

| | Relative | | Relative |
|---|---|---|---|
| | Expression(%) | | Expression(%) |
| | 2dx4tm4720f_{—} | | 2dx4tm4720f_{—} |
| Tissue Name | ag1391_a1 | Tissue Name | ag1391_a1 |
| Normal Colon GENPAK | 20.9 | Kidney NAT Clontech 8120608 | 3.4 |
| 061003 | | | |
| 83219 CC Well to Mod Diff | 18.6 | Kidney Cancer Clontech | 1.2 |
| (ODO3866) | | 8120613 | |
| 83220 CC NAT (ODO3866) | 5.4 | Kidney NAT Clontech 8120614 | 7.6 |
| 83221 CC Gr.2 rectosigmoid | 2.3 | Kidney Cancer Clontech | 5.8 |
| (ODO3868) | | 9010320 | |
| 83222 CC NAT ODO3868) | 4.5 | Kidney NAT Clontech 9010321 | 12.8 |
| 83235 CC Mod Diff | 4.7 | Normal Uterus GENPAK | 3.8 |
| (ODO3920) | | 061018 | |
| 83236 CC NAT (ODO3920) | 6.8 | Uterus Cancer GENPAK | 11.8 |
| | | 064011 | |
| 83237 CC Gr.2 ascend colon | 3.4 | Normal Thyroid Clontech A+ | 100.0 |
| (ODO3921) | | 6570-1 | |
| 83238 CC NAT (ODO3921) | 2.5 | Thyroid Cancer GENPAK | 4.3 |
| | | 064010 | |
| 83241 CC from Partial | 4.2 | Thyroid Cancer INVITROGEN | 8.6 |
| Hepatectomy (ODO4309) | | A302152 | |
| 83242 Liver NAT (ODO4309) | 1.0 | Thyroid NAT INVITROGEN | 55.4 |
| | | A302153 | |
| 87472 Colon mets to lung | 3.5 | Normal Breast GENPAK | 11.1 |
| (OD04451-01) | | 061019 | |
| 87473 Lung NAT (OD04451-02) | 3.3 | 84877 Breast Cancer | 6.1 |
| | | (OD04566) | |
| Normal Prostate Clontech A+ 6546-1 | 26.3 | 85975 Breast Cancer | 11.4 |
| | | (OD04590-01) | |
| 84140 Prostate Cancer | 11.4 | 85976 Breast Cancer Mets | 19.6 |
| (OD04410) | | (OD04590-03) | |
| 84141 Prostate NAT | 26.1 | 87070 Breast Cancer Metastasis | 6.4 |
| (OD04410) | | (OD04655-05) | |
| 87073 Prostate Cancer | 12.4 | GENPAK Breast Cancer | 18.5 |
| (OD04720-01) | | 064006 | |
| 87074 Prostate NAT | 21.3 | Breast Cancer Res. Gen. 1024 | 11.9 |
| (OD04720-02) | | | |
| Normal Lung GENPAK 061010 | 19.3 | Breast Cancer Clontech | 9.5 |
| | | 9100266 | |
| 83239 Lung Met to Muscle | 1.8 | Breast NAT Clontech 9100265 | 5.7 |
| (ODO4286) | | | |
| 83240 Muscle NAT | 2.5 | Breast Cancer INVITROGEN | 9.1 |
| (ODO4286) | | A209073 | |
| 84136 Lung Malignant Cancer | 3.4 | Breast NAT INVITROGEN | 6.0 |
| (OD03126) | | A2090734 | |
| 84137 Lung NAT (OD03126) | 12.0 | Normal Liver GENPAK | 1.2 |
| | | 061009 | |
| 84871 Lung Cancer (OD04404) | 4.2 | Liver Cancer GENPAK 064003 | 1.2 |
| 84872 Lung NAT (OD04404) | 5.6 | Liver Cancer Research Genetics | 2.1 |
| | | RNA 1025 | |
| 84875 Lung Cancer (OD04565) | 5.4 | Liver Cancer Research Genetics | 1.5 |
| | | RNA 1026 | |
| 84876 Lung NAT (OD04565) | 5.5 | Paired Liver Cancer Tissue | 1.5 |
| | | Research Genetics RNA 6004-T | |
| 85950 Lung Cancer (OD04237-01) | 10.3 | Paired Liver Tissue Research | 3.7 |
| | | Genetics RNA 6004-N | |
| 85970 Lung NAT (OD04237-02) | 6.9 | Paired Liver Cancer Tissue | 0.8 |
| | | Research Genetics RNA 6005-T | |
| 83255 Ocular Mel Met to Liver | 6.3 | Paired Liver Tissue Research | 0.5 |
| (ODO4310) | | Genetics RNA 6005-N | |
| 83256 Liver NAT (ODO4310) | 0.8 | Normal Bladder GENPAK | 6.8 |
| | | 061001 | |
| 84139 Melanoma Mets to Lung | 6.9 | Bladder Cancer Research | 27.6 |
| (OD04321) | | Genetics RNA 1023 | |
| 84138 Lung NAT (OD04321) | 5.6 | Bladder Cancer INVITROGEN | 9.4 |
| | | A302173 | |
| Normal Kidney GENPAK | 25.8 | 87071 Bladder Cancer | 3.0 |
| 061008 | | (OD04718-01) | |
| 83786 Kidney Ca, Nuclear | 12.6 | 87072 Bladder Normal | 41.9 |
| grade 2p12 (OD04338) | | Adjacent (OD04718-03) | |
| 83787 Kidney NAT (OD04338) | 14.9 | Normal Ovary Res. Gen. | 5.0 |
| 83788 Kidney Ca Nuclear grade | 11.3 | Ovarian Cancer GENPAK | 10.9 |
| 1/2 (OD04339) | | 064008 | |
| 83789 Kidney NAT (OD04339) | 16.6 | 87492 Ovary Cancer | 10.7 |
| | | (OD04768-07) | |
| 83790 Kidney Ca, Clear cell | 11.5 | 87493 Ovary NAT (OD04768- | 1.7 |
| type (OD04340) | | 08) | |
| 83791 Kidney NAT (OD04340) | 21.9 | Normal Stomach GENPAK | 10.8 |
| | | 061017 | |
| 83792 Kidney Ca, Nuclear | 2.9 | Gastric Cancer Clontech | 3.4 |
| grade 3 (OD04348) | | 9060358 | |
| 83793 Kidney NAT (OD04348) | 11.8 | NAT Stomach Clontech | 2.4 |
| | | 9060359 | |
| 87474 Kidney Cancer | 2.0 | Gastric Cancer Clontech | 2.7 |
| (OD04622-01) | | 9060395 | |
| 87475 Kidney NAT (OD04622-03) | 3.9 | NAT Stomach Clontech | 3.9 |
| | | 9060394 | |
| 85973 Kidney Cancer | 3.2 | Gastric Cancer Clontech | 6.3 |
| (OD04450-01) | | 9060397 | |
| 85974 Kidney NAT (OD04450-03) | 11.9 | NAT Stomach Clontech | 1.5 |
| | | 9060396 | |
| Kidney Cancer Clontech | 1.9 | Gastric Cancer GENPAK | 10.0 |
| 8120607 | | 064005 | |

TABLE AH
Panel 4D

| | | | Relative |
|---|---|---|---|
| | Relative Expression(%) | | Expression(%) |
| | 4dtm2440f_ag- | 4Dtm2506f_ag- | 4dx4tm5044f_{—} |
| Tissue Name | 1391 | 1391 | ag2890_b1 |
| 93768_Secondary Th1_anti-CD28/anti-CD3 | 1.9 | 4.5 | 1.2 |
| 93769_Secondary Th2_anti-CD28/anti-CD3 | 3.0 | 3.9 | 2.1 |
| 93770_Secondary Tr1_anti-CD28/anti-CD3 | 2.1 | 3.5 | 2.0 |
| 93573_Secondary Th1_resting day 4-6 in IL-2 | 0.3 | 0.6 | 0.4 |
| 93572_Secondary Th2_resting day 4-6 in IL-2 | 0.7 | 0.8 | 0.8 |
| 93571_Secondary Tr1_resting day 4-6 in IL-2 | 0.8 | 3.8 | 0.5 |
| 93568_primary Th1_anti-CD28/anti-CD3 | 2.1 | 2.3 | 1.0 |
| 93569_primary Th2_anti-CD28/anti-CD3 | 2.1 | 3.4 | 1.7 |
| 93570_primary Tr1_anti-CD28/anti-CD3 | 4.0 | 2.1 | 1.8 |
| 93565_primary Th1_resting dy 4-6 in IL-2 | 2.9 | 4.4 | 4.1 |
| 93566_primary Th2_resting dy 4-6 in IL-2 | 2.1 | 2.8 | 2.0 |
| 93567_primary Tr1_resting dy 4-6 in IL-2 | 2.8 | 2.3 | 2.6 |
| 93351_CD45RA CD4 lymphocyte_anti- | 21.8 | 25.5 | 16.5 |
| CD28/anti-CD3 | | | |
| 93352_CD45RO CD4 lymphocyte_anti- | 2.8 | 3.0 | 1.3 |
| CD28/anti-CD3 | | | |
| 93251_CD8 Lymphocytes_anti-CD28/anti-CD3 | 1.1 | 2.0 | 1.7 |
| 93353_chronic CD8 Lymphocytes 2ry_resting | 1.9 | 2.5 | 1.6 |
| dy 4-6 in IL-2 | | | |
| 93574_chronic CD8 Lymphocytes 2ry_activated | 1.6 | 0.8 | 1.2 |
| CD3/CD28 | | | |
| 93354_CD4_none | 0.8 | 0.6 | 0.8 |
| 93252_Secondary Th1/Th2/Tr1_anti-CD95 | 3.1 | 2.0 | 1.7 |
| CH11 | | | |
| 93103_LAK cells_resting | 1.9 | 2.6 | 1.5 |
| 93788_LAK cells IL-2 | 4.7 | 3.0 | 2.4 |
| 93787_LAK cells_IL-2 + IL-12 | 2.6 | 1.6 | 2.1 |
| 93789_LAK cells_IL-2 + IFN gamma | 3.2 | 7.0 | 3.9 |
| 93790_LAK cells_IL-2 + IL-18 | 2.2 | 4.3 | 3.3 |
| 93104_LAK cells_PMA/ionomycin and IL-18 | 1.0 | 1.0 | 1.3 |
| 93578_NK Cells IL-2_resting | 1.9 | 3.5 | 2.5 |
| 93109_Mixed Lymphocyte Reaction_Two Way | 1.4 | 2.2 | 2.9 |
| MLR | | | |
| 93110_Mixed Lymphocyte Reaction_Two Way | 1.1 | 1.8 | 1.8 |
| MLR | | | |
| 93111_Mixed Lymphocyte Reaction_Two Way | 1.3 | 1.1 | 1.4 |
| MLR | | | |
| 93112_Mononuclear Cells (PBMCs)_resting | 0.3 | 1.1 | 0.2 |
| 93113_Mononuclear Cells (PBMCs)_PWM | 5.6 | 7.4 | 6.7 |
| 93114_Mononuclear Cells (PBMCs)_PHA-L | 3.3 | 3.3 | 1.8 |
| 93249_Ramos (B cell)_none | 5.9 | 3.4 | 2.9 |
| 93250_Ramos (B cell)_ionomycin | 8.8 | 9.0 | 7.8 |
| 93349_B lymphocytes_PWM | 3.1 | 4.4 | 3.4 |
| 93350_B lymphoytes_CD40L and IL-4 | 3.4 | 3.4 | 3.7 |
| 92665_EOL-1 (Eosinophil)_dbcAMP | 2.6 | 5.7 | 3.5 |
| differentiated | | | |
| 93248_EOL-1 | 5.6 | 7.3 | 5.1 |
| (Eosinophil)_dbcAMP/PMAionomycin | | | |
| 93356_Dendritic Cells_none | 1.0 | 1.0 | 0.8 |
| 93355_Dendritic Cells_LPS 100 ng/ml | 1.4 | 1.6 | 1.2 |
| 93775_Dendritic Cells_anti-CD40 | 0.9 | 1.4 | 0.8 |
| 93774_Monocytes_resting | 1.0 | 1.7 | 2.0 |
| 93776_Monocytes_LPS 50 ng/ml | 4.1 | 7.0 | 2.5 |
| 93581_Macrophages_resting | 1.7 | 1.8 | 1.3 |
| 93582_Macrophages_LPS 100 ng/ml | 3.0 | 1.5 | 0.8 |
| 93098_HUVEC (Endothelial)_none | 2.5 | 3.9 | 3.1 |
| 93099_HUVEC (Endothelial)_starved | 4.8 | 10.5 | 4.8 |
| 93100_HUVEC (Endothelial)_IL-1b | 2.1 | 2.4 | 1.6 |
| 93779_HUVEC (Endothelial)_IFN gamma | 4.3 | 6.2 | 3.7 |
| 93102_HUVEC (Endothelial)_TNF alpha + IFN | 1.1 | 2.4 | 1.9 |
| gamma | | | |
| 93101_HUVEC (Endothelial)_TNF alpha + IL4 | 1.7 | 3.1 | 2.7 |
| 93781_HUVEC (Endothelial)_IL-11 | 3.8 | 3.3 | 3.9 |
| 93583_Lung Microvascular Endothelial | 6.0 | 17.3 | 7.3 |
| Cells_none | | | |
| 93584_Lung Microvascular Endothelial | 5.0 | 8.8 | 6.3 |
| Cells_TNFa (4 ng/ml) and IL1b (1 ng/ml) | | | |
| 92662_Microvascular Dermal | 11.8 | 12.9 | 6.8 |
| endothelium_none | | | |
| 92663_Microsvasular Dermal | 4.2 | 3.9 | 3.9 |
| endothelium_TNFa (4 ng/ml) and IL1b (1 ng/ml) | | | |
| 93773_Bronchial epithelium_TNFa (4 ng/ml) | 1.0 | 1.2 | 1.3 |
| and IL1b (1 ng/ml)** | | | |
| 93347_Small Airway Epithelium_none | 0.1 | 0.5 | 0.9 |
| 93348_Small Airway Epithelium_TNFa (4 ng/ml) | 2.9 | 4.0 | 2.8 |
| and IL1b (1 ng/ml) | | | |
| 92668_Coronery Artery SMC_resting | 4.5 | 5.5 | 3.4 |
| 92669_Coronery Artery SMC_TNFa (4 ng/ml) | 2.3 | 1.5 | 1.3 |
| and IL1b (1 ng/ml) | | | |
| 93107_astrocytes_resting | 4.8 | 8.2 | 6.0 |
| 93108_astrocytes_TNFa (4 ng/ml) and IL1b (1 ng/ml) | 10.0 | 12.6 | 10.6 |
| 92666_KU-812 (Basophil)_resting | 2.5 | 3.8 | 2.3 |
| 92667_KU-812 (Basophil)_PMA/ionoycin | 5.4 | 10.7 | 6.3 |
| 93579_CCD1106 (Keratinocytes)_none | 1.0 | 3.1 | 2.5 |
| 93580_CCD1106 (Keratinocytes)_TNFa and | 1.3 | 0.7 | 0.6 |
| IFNg** | | | |
| 93791_Liver Cirrhosis | 1.1 | 1.7 | 0.9 |
| 93792_Lupus Kidney | 9.0 | 7.5 | 2.7 |
| 93577_NCI-H292 | 3.7 | 4.7 | 3.7 |
| 93358_NCI-H292_IL-4 | 5.8 | 11.7 | 6.4 |
| 93360_NCI-H292_IL-9 | 5.3 | 9.4 | 5.4 |
| 93359_NCI-H292_IL-13 | 4.1 | 5.1 | 3.5 |
| 93357_NCI-H292_IFN gamma | 2.5 | 4.7 | 3.5 |
| 93777_HPAEC_- | 3.4 | 2.9 | 1.6 |
| 93778_HPAEC_IL-1 beta/TNA alpha | 2.8 | 4.0 | 2.2 |
| 93254_Normal Human Lung Fibroblast_none | 2.6 | 2.9 | 2.3 |
| 93253_Normal Human Lung Fibroblast_TNFa | 1.4 | 1.0 | 2.0 |
| (4 ng/ml) and IL-1b (1 ng/ml) | | | |
| 93257_Normal Human Lung Fibroblast_IL-4 | 10.6 | 17.0 | 13.0 |
| 93256_Normal Human Lung Fibroblast_IL-9 | 2.2 | 4.3 | 4.1 |
| 93255_Normal Human Lung Fibroblast_IL-13 | 17.3 | 10.1 | 9.9 |
| 93258_Normal Human Lung Fibroblast_IFN | 3.5 | 4.0 | 3.8 |
| gamma | | | |
| 93106_Dermal Fibroblasts CCD1070_resting | 100.0 | 100.0 | 100.0 |
| 93361_Dermal Fibroblasts CCD1070_TNF | 93.3 | 91.4 | 94.1 |
| alpha 4 ng/ml | | | |
| 93105_Dermal Fibroblasts CCD1070_IL-1 beta | 41.5 | 33.9 | 35.5 |
| 1 ng/ml | | | |
| 93772_dermal fibroblast_IFN gamma | 3.3 | 4.0 | 4.7 |
| 93771_dermal fibroblast_IL-4 | 18.3 | 20.2 | 13.9 |
| 93259_IBD Colitis 1** | 1.4 | 0.5 | 1.2 |
| 93260_IBD Colitis 2 | 0.7 | 0.5 | 0.3 |
| 93261_IBD Crohns | 0.3 | 0.4 | 0.3 |
| 735010_Colon_normal | 3.0 | 4.2 | 1.8 |
| 735019_Lung_none | 1.8 | 3.3 | 2.4 |
| 64028-1_Thymus_none | 24.1 | 16.8 | 13.0 |
| 64030-1_Kidney_none | 10.7 | 13.0 | 11.4 |

Panel 1 Summary: Ag192 Results from two experiments using the same probe/primer set show fair agreement, although expression levels are higher and more ubiquitous in one of the experiments. In both runs, the NOV8 gene is most highly expressed by astrocytoma cell line SNB-75 (CT=24). Thus, based upon the strongest expression in astrocytoma cells, the expression of this gene might be of use in the diagnosis of astrocytoma. In addition, the therapeutic down-modulation of the activity of the NOV8 gene product, through the use of small molecule drugs or antibodies, may be useful in the treatment of astrocytoma.

This gene is also expressed at moderate to high amounts throughout the CNS with expression detected in amygdala, hippocampus, substantia nigra, thalamus, cerebellum, hypothalamus and spinal cord. Please see Panel 1.3D Summary for discussion of the potential relevance of this expression pattern.

Panel 1.1 Summary: Ag192 Expression of the NOV8 gene in this panel is highest in a sample derived from thyroid tissue (CT=25). There is also significant expression of this gene in samples derived from a prostate cancer cell line (PC-3), a melanoma cell line (Hs688(B)), a lung cancer cell line (NCI-H522) and an astrocytoma cell line (SNB-75). Thus, based upon this pattern of gene expression, the expression of the NOV8 gene might be of use as a marker of normal thymic tissue. In addition, therapeutic down-modulation of the NOV8 gene product, through the use of small molecule inhibitors or antibodies, might be of use in the treatment of the above listed cancer types.

The NOV8 gene has modest levels of expression in pituitary, liver, skeletal and fetal muscle, adrenal gland and pancreas (CT values 28-32) and higher expression in heart (CT=26). Interestingly, NOV8 gene expression differs between fetal (CT=40) and adult liver (CT=31), suggesting that this gene product may be useful for differentiating between the fetal and adult liver.

Panel 1.2 Summary: Ag1391 The expression of the NOV8 gene as assessed in two independent runs in Panel 1.2 is similar to the results above; please see Panel 11.1 surumary for discussion of results. However, the highest expression in one run is in the lung cancer cell line NCI-H522 and in the repeat run is the astrocytoma cell line SNB-75.

The NOV8 gene has modest expression in pancreas (CT=30-31), pituitary (CT=30-33), skeletal muscle (CT=28-29), and fetal and adult liver (CT=29-31). In contrast to what is observed on Panel 1.1, there is no significant difference in expression between fetal and adult liver on this panel. This gene also has high expression in adrenal (CT=25-26), thyroid (CT=25-28), and heart (CT=24), suggesting that the NOV8 gene product may be a drug target for any or all diseases involving these tissues.

Panel 1.3D Summary: Ag2890 The expression of the NOV8 gene in this panel is highest in a sample derived from a melanoma cell line (Hs688(B)). Of note is the fact that this cell line was derived from a metastatic melanoma while a representative cell line of the primary melanoma is also found on panel 1.3D (Hs688(A)). The expression of this gene in the Hs688(A) sample is markedly lower relative to Hs688(B). Thus, expression of the NOV8 gene might be of use in the detection or diagnosis of metastatic melanoma when compared to primary melanoma. In addition, the therapeutic down modulation of the NOV8 gene product, through the use of small molecule drugs or antibodies, might be of utility as a treatment of metastatic melanoma.

The NOV8 gene is expressed at low to moderate levels in pancreas, adrenal, thyroid, pituitary, fetal and adult heart, fetal and adult skeletal muscle, and fetal and adult liver and may therefore play a role in diseases involving any or all of these tissues.

Interestingly, the NOV8 gene is more highly expressed in fetal brain (CT=30.7) vs. adult brain (CT=32.1). The function of the MEGF/Fibrillin protein family is not completely understood, but expression in the fetal brain suggests a role in neurodevelopment, especially in the development of axonal pathways. In CNS diseases and trauma where compensatory sprouting and establishment of new connections would be appropriate in response to neuronal death (e.g., Alzheimer's, Parkinson's, Huntington's, spinocerebellar ataxia, head/spinal cord trauma, stroke, etc.) the therapeutic modulation of the NOV8 gene or its protein product may be beneficial to the establishment of proper neuronal connectivity during induced neurogenesis, axonal/dendritic outgrowth, synaptogenesis, or stem cell treatment.

Panel 2D Summary: Ag1391 Expression of the NOV8 gene is highest in normal thyroid tissue and in a second sample derived from the matched normal margin of a thyroid cancer. Based upon this expression profile, the expression of this gene could be used to distinguish normal thyroid tissue from malignant thyroid tissue. In addition, the therapeutic replacement of the NOV8 gene product in thyroid cancer might be of use in the treatment of this disease.

Panel 4D Summary: Ag1391/Ag2890 Results from three experiments using the same probe/primer set are in good agreement. Expression of the NOV8 gene is restricted to dermal fibroblasts; levels of expression are similar under resting conditions as well as after stimulation with either TNF-alpha or IL-1-beta. The NOV8 gene is predicted encode a MEGF/fibrillin membrane protein that may localize to the plasma membrane. Therefore, antibodies raised against the NOV8 gene product may be useful therapeutics in skin diseases. Alternatively, the isolated extracellular domain of the NOV8 protein may function as a therapeutic protein in the treatment of skin diseases.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18 and 20;
(b) a variant of a mature form of an amino acid sequence selected from the group consisting of SEQ ID NOS:2, 4, 6, 8, 10, 12, 14, 16, 18 and 20, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS:2, 4, 6, 8, 10, 12, 14, 16, 18 and 20; and
(d) a variant of an amino acid sequence selected from the group consisting of SEQ ID NOS:2, 4, 6, 8, 10, 12, 14, 16, 18 and 20, wherein one or more amino acid residues in said variant differs from the amino acid sequence of said mature form, provided that said variant differs in no more than 15% of amino acid residues from said amino acid sequence.

2 The polypeptide of claim 1, wherein said polypeptide comprises the amino acid sequence of a naturally-occurring allelic variant of an amino acid sequence selected from the group consisting SEQ ID NOS:2, 4, 6, 8, 10, 12, 14, 16, 18 and 20.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, and 19.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18 and 20;
(b) a variant of a mature form of an amino acid sequence selected from the group consisting of SEQ ID NOS:2, 4, 6, 8, 10, 12, 14, 16, 18 and 20, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18 and 20;
(d) a variant of an amino acid sequence selected from the group consisting SEQ ID NOS:2, 4, 6, 8, 10, 12, 14, 16, 18 and 20, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18 and 20, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, and 19.

9. The nucleic acid molecule of claim 5, wherein said nucleic acid molecule comprises a nucleotide sequence selected from the group consisting of:
(a) a nucleotide sequence selected from the group consisting of SEQ ID NOS:1, 3, 5, 7, 9, 11, 13, 15, 17, and 19;
(b) a nucleotide sequence differing by one or more nucleotides from a nucleotide sequence selected from the group consisting of SEQ ID NOS:1, 3, 5, 7, 9, 11, 13, 15, 17, and 19, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS:1, 3, 5, 7, 9, 11, 13, 15, 17, and 19, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18 and 20, or a biologically active fragment thereof.

49. A method of treating a pathological state in a mammal, the method comprising administering to the mammal the antibody of claim 15 in an amount sufficient to alleviate the pathological state.
